(19) United States
(12) Patent Application Publication
(10) Pub. No.: US 2022/0017979 A1
(43) Pub. Date: Jan. 20, 2022

(54) METHODS OF DETECTING SARS-COV-2, INFLUENZA, AND RSV

(71) Applicant: Cepheid, Sunnyvale, CA (US)

(72) Inventors: Victor Chu, Santa Clara, CA (US); Sergey Lokhov, Bothell, WA (US); Oliver Nanassy, Edmonds, WA (US); Richard Joseph Leuzzi, Seattle, WA (US); Jun Wang, Santa Clara, CA (US)

(73) Assignee: Cepheid, Sunnyvale, CA (US)

(21) Appl. No.: 17/358,523

(22) Filed: Jun. 25, 2021

Related U.S. Application Data
(60) Provisional application No. 63/044,902, filed on Jun. 26, 2020.
(60) Provisional application No. 63/074,809, filed on Sep. 4, 2020.

(51) Int. Cl. C12Q 1/70

(52) U.S. Cl. CPC C12Q 1/701; C12Q 2600/158

(57) ABSTRACT

Compositions and methods for detecting Severe Acute Respiratory Syndrome Coronavirus 2 (SARS-CoV-2), influenza A, influenza B, and respiratory syncytial virus (RSV) are provided.

### 1. CROSS-REFERENCE TO RELATED APPLICATIONS

This application claims the benefit of priority of U.S. Provisional Application No. 63/044,902, filed Jun. 26, 2020, and U.S. Provisional Application No. 63/074,809, filed Sep. 4, 2020, each of which is incorporated by reference herein in its entirety for any purpose.

### 2. FIELD OF THE INVENTION

Compositions and methods for detecting Severe Acute Respiratory Syndrome Coronavirus 2 (SARS-CoV-2), influenza, and respiratory syncytial virus (RSV) are provided. In particular, SARS-CoV-2, influenza, and RSV markers and panels of markers useful in the detection of SARS-CoV-2, influenza, and RSV virus are provided.

### 3. BACKGROUND

On Dec. 31 2019, an outbreak of respiratory illness of unknown etiology was reported to the World Health Organization (WHO). A novel coronavirus (2019-nCoV) was identified, which has resulted in thousands of confirmed human infections in multiple provinces throughout the world. Cases of severe illness and some deaths have been reported. The International Committee for Taxonomy of Viruses (ICTV) renamed the virus SARS-CoV-2, which is short for “Severe Acute Respiratory Syndrome Coronavirus 2.” The World Health Organization has named the disease caused by the SARS-CoV-2 as coronavirus disease 2019 (COVID-19). COVID-19 is associated with a variety of clinical outcomes, including asymptomatic infection, mild upper respiratory infection, severe lower respiratory disease including pneumonia and respiratory failure, and in some cases, death. According to the Center for Disease Control and Prevention (U.S. CDC), as of June 2020, it was known that patients with COVID-19 exhibit a wide range of symptoms—ranging from mild symptoms to severe illness. Symptoms, including but not limited to fever or chills, cough, shortness of breath or difficulty breathing, fatigue, muscle or body aches, headache, new loss of taste or smell, sore throat, congestion or runny nose, nausea or vomiting, and/or diarrhea, may appear 2-14 days after exposure to the virus.

Influenza, or the flu, is a contagious viral infection of the respiratory tract. Transmission of influenza is primarily airborne (i.e., coughing or sneezing); the peak of transmission usually occurs in the winter months. Symptoms commonly include fever, chills, headache, muscle aches, malaise, cough, and sinus congestion. Gastrointestinal symptoms (i.e., nausea, vomiting, or diarrhea) may also occur, primarily in children, but are less common in adults. Symptoms generally appear within two days of exposure to an infected person. Pneumonia may develop as a complication of influenza infection, causing increased morbidity and mortality in pediatric, elderly, and immunocompromised populations. Influenza viruses are classified into types A, B, and C, the former two of which cause most human infections. Influenza A is the most common type of influenza virus in humans, and is generally responsible for seasonal flu epidemics and occasionally for pandemics. Influenza A viruses can also infect animals such as birds, pigs, and horses. Infections with influenza B virus are generally restricted to humans and are less frequent causes of epidemics. Influenza A viruses are further divided into subtypes on the basis of two surface proteins: hemagglutinin (H) and neuraminidase (N). Seasonal flu is normally caused by subtypes H1, H2, H3, and N1 and N2. In addition to seasonal flu, a novel H1N1 strain was identified in humans in the United States in early 2009.

Respiratory syncytial virus (RSV), a member of the Pneumoviridae family (formerly Paramyxoviridae family) consisting of two strains (subgroups A and B), is also the cause of a contagious disease that afflicts primarily infants and the elderly who are immune-compromised, e.g., chronic lung or heart disease or undergoing treatment for conditions that reduces the strength of their immune system. The virus can live for hours on countertops and toys and cause both upper respiratory infections, such as colds, and lower respiratory infections manifesting as bronchiolitis and pneumonia. By the age of two, most children have already been infected by RSV, but because only weak immunity develops, both children and adults can become reinfected. Symptoms usually appear four to six days after infection. The disease is typically self-limiting, lasting about one to two weeks in infants. In adults, the infection lasts about five days and presents with symptoms consistent with a cold, such as rhinorrhea, fatigue, headache, and fever. The RSV season overlaps with influenza season somewhat as infections begin to rise during the fall and continue through early spring. RSV infections, however, also occur at other times of the year, although rarely.

Active surveillance programs in conjunction with infection control precautions are important components for preventing transmission of SARS-CoV-2, influenza, and RSV. The use of assays providing rapid results to identify patients infected with these infections is also an important factor for effective control, proper choice of treatment, and prevention of widespread outbreaks.

The genome of influenza viruses comprises eight RNA segments of 0.9-2.3 kb that together span approximately 13.5 kb and encode 11 proteins. These 8 segments designated PB2, PB1, PA, HA, NP, NA, MP and NS are under constant selective pressure which leads to rapid sequence changes (antigenic drift). In addition to changes on the sequence level Influenza A has the ability to exchange whole segments with other Influenza A viruses (antigenic shift).

This process leads to the emergence of pandemic influenza strains (i.e. Influenza A H1N1pdm09, swine origin H3N2).

The two proteins, hemagglutinin (HA) and neuraminidase (NA) determine the subtypes (H and N, respectively) of Influenza A virus. There are 16 H subtypes and 9 N subtypes. The H1N1 and H3N2 subtypes cause the vast majority of influenza infections in humans. Influenza B virus has a similar structure of RNA segments; however the Flu B viruses do not have subtypes.

This constant antigenic drift and antigenic shift makes it difficult to maintain influenza detection assays from season to season. Additionally, there is a need for a next-generation test to assist global efforts in the fight against the spread of COVID-19, especially during future respiratory virus seasons. There is a need for a robust SARS-CoV-2, influenza, and RSV detection assay that will remain accurate even as the viral genomes undergo genetic drift. Patients with COVID-19, Flu, and RSV have overlapping clinical presentations, but fundamentally different treatment and management pathways. Infection with the viruses is often associated with fever and other systemic manifestations that may be coupled with severe outcomes, especially in the elderly. There is thus a need and demand for a test that can deliver qualitative detection and differentiation of SARS-CoV-2, Flu A, Flu B, and RSV from a single patient sample. Furthermore, obtaining results in a short amount of time is beneficial.

### 4. SUMMARY

In some instances, the following non-limiting embodiments are provided:
Embodiment 1. A method of detecting the presence or absence of influenza A, influenza B, RSV, and SARS-CoV-2 in a biological sample from a subject comprising:
   a) contacting a biological sample from the subject with sets of primers that detect an influenza A gene, an influenza B gene, a RSV gene, and a SARS-CoV-2 gene;
   b) conducting one or more polymerase chain reaction (PCR); and
   c) detecting an amplicon that is produced by the PCR.
Embodiment 2. A method of determining whether a subject has influenza, RSV, and/or COVID-19 comprising detecting the presence or absence of at least one gene selected from influenza A or influenza B, RSV, and SARS-CoV-2 in a sample from the subject comprising:
   a) contacting a biological sample from the subject with sets of primers that detect an influenza A gene, an influenza B gene, an RSV gene, and a SARS-CoV-2 gene;
   b) conducting a polymerase chain reaction (PCR); and
   c) detecting an amplicon that is produced by the PCR.
Embodiment 3. The method of embodiment 1 or embodiment 2,
   a) wherein the set of primers that detects the presence or absence of influenza A comprises at least one of:
      i) a set of primers that detects influenza A PB2 selected from:
         1) a forward and reverse primer for detecting a sequence of the influenza PB2 gene;
         2) a forward primer comprising a sequence that is at least 85% identical to at least 15 contiguous nucleotides of SEQ ID NO: 1, and a reverse primer comprising a sequence that is at least 85% complementary to at least 15 contiguous nucleotides of SEQ ID NO: 1; and
         3) a forward primer comprising a sequence that is at least 85% identical to at least 15 contiguous nucleotides of SEQ ID NO: 17, and a reverse primer comprising a sequence that is at least 85% identical to at least 15 contiguous nucleotides of SEQ ID NO: 18;
      ii) a set of primers that detects influenza A PA selected from:
         1) a forward and reverse primer for detecting a sequence of the influenza PA gene;
         2) a forward primer comprising a sequence that is at least 85% identical to at least 15 contiguous nucleotides of SEQ ID NO: 2, and a reverse primer comprising a sequence that is at least 85% complementary to at least 15 contiguous nucleotides of SEQ ID NO: 2; and
         3) a forward primer comprising a sequence that is at least 85% identical to at least 15 contiguous nucleotides of SEQ ID NO: 20, and a reverse primer comprising a sequence that is at least 85% identical to at least 15 contiguous nucleotides of SEQ ID NO: 21;
      iii) a set of primers that detects influenza A MP selected from:
         1) a forward and reverse primer for detecting a sequence of the influenza A MP gene;
         2) a forward primer comprising a sequence that is at least 85% identical to at least 15 contiguous nucleotides of SEQ ID NO: 3, and a reverse primer comprising a sequence that is at least 85% complementary to at least 15 contiguous nucleotides of SEQ ID NO: 3; and
         3) a forward primer comprising a sequence that is at least 85% identical to at least 15 contiguous nucleotides of SEQ ID NO: 23, and a reverse primer comprising a sequence that is at least 85% identical to at least 15 contiguous nucleotides of SEQ ID NO: 24;
      iv) a set of primers that detects avian influenza MP selected from:
         1) a forward and reverse primer for detecting a sequence of the avian influenza MP gene;
         2) a forward primer comprising a sequence that is at least 85% identical to at least 15 contiguous nucleotides of SEQ ID NO: 4, and a reverse primer comprising a sequence that is at least 85% complementary to at least 15 contiguous nucleotides of SEQ ID NO: 4; and
         3) a forward primer comprising a sequence that is at least 85% identical to at least 15 contiguous nucleotides of SEQ ID NO: 26, and a reverse primer comprising a sequence that is at least 85% identical to at least 15 contiguous nucleotides of SEQ ID NO: 27;
   b) wherein the set of primers that detects the presence or absence of influenza B comprises at least one of:
      i) a set of primers that detects influenza B MP selected from:
         1) a forward and reverse primer for detecting a sequence of the influenza B MP gene;
         2) a forward primer comprising a sequence that is at least 85% identical to at least 15 contiguous nucleotides of SEQ ID NO: 6, and a reverse primer comprising a sequence that is at least 85% complementary to at least 15 contiguous nucleotides of SEQ ID NO: 6; and
         3) a forward primer comprising a sequence that is at least 85% identical to at least 15 contiguous nucleotides of SEQ ID NO: 32, and a reverse primer comprising a sequence that is at least 85% identical to at least 15 contiguous nucleotides of SEQ ID NO: 33;
      ii) a set of primers that detects influenza B NS selected from:
         1) a forward and reverse primer for detecting a sequence of the influenza B NS gene;
         2) a forward primer comprising a sequence that is at least 85% identical to at least 15 contiguous nucleotides of SEQ ID NO: 7, and a reverse primer comprising a sequence that is at least 85% complementary to at least 15 contiguous nucleotides of SEQ ID NO: 7; and
         3) a forward primer comprising a sequence that is at least 85% identical to at least 15 contiguous nucleotides of SEQ ID NO: 35, and a reverse primer comprising a sequence that is at least 85% identical to at least 15 contiguous nucleotides of SEQ ID NO: 36;
   c) wherein the set of primers that detects the presence or absence of RSV comprises at least one of:
      i) a set of primers that detects RSV A selected from:
         1) a forward and reverse primer for detecting a sequence of the RSV A gene; and
         2) a forward primer comprising a sequence that is at least 85% identical to at least 15 contiguous nucleotides of SEQ ID NO: 38, and a reverse primer comprising a sequence that is at least 85% complementary to at least 15 contiguous nucleotides of SEQ ID NO: 39; and
      ii) a set of primers that detects RSV B selected from:
         1) a forward and reverse primer for detecting a sequence of the RSV B gene; and
         2) a forward primer comprising a sequence that is at least 85% identical to at least 15 contiguous nucleotides of SEQ ID NO: 41, and a reverse primer comprising a sequence that is at least 85% identical to at least 15 contiguous nucleotides of SEQ ID NO: 42; and
   d) wherein the set of primers that detects the presence or absence of SARS-CoV-2 comprises at least one of:
      i) a set of primers that detects SARS-CoV-2 E selected from:
         1) a forward and reverse primer for detecting a sequence of the SARS-CoV-2 E gene;
         2) a forward primer comprising a sequence that is at least 85% identical to at least 15 contiguous nucleotides of SEQ ID NO: 44, and a reverse primer comprising a sequence that is at least 85% complementary to at least 15 contiguous nucleotides of SEQ ID NO: 44; and
         3) a forward primer comprising a sequence that is at least 85% identical to at least 15 contiguous nucleotides of SEQ ID NO: 48, and a reverse primer comprising a sequence that is at least 85% identical to at least 15 contiguous nucleotides of SEQ ID NO: 49; and
      ii) a set of primers that detects SARS-CoV-2 N2 selected from:
         1) a forward and reverse primer for detecting a sequence of the SARS-CoV-2 N2 gene;
         2) a forward primer comprising a sequence that is at least 85% identical to at least 15 contiguous nucleotides of SEQ ID NO: 45, and a reverse primer comprising a sequence that is at least 85% complementary to at least 15 contiguous nucleotides of SEQ ID NO: 45; and
         3) a forward primer comprising a sequence that is at least 85% identical to at least 15 contiguous nucleotides of SEQ ID NO: 51, and a reverse primer comprising a sequence that is at least 85% identical to at least 15 contiguous nucleotides of SEQ ID NO: 52.
Embodiment 4. The method of embodiment 1 or embodiment 2,
   a) wherein the set of primers that detects the presence or absence of influenza A comprises at least one of:
      i) a set of primers that detects influenza A PB2 selected from:
         1) a forward and reverse primer for detecting a sequence of the influenza PB2 gene;
         2) a forward primer comprising a sequence that is at least 85% identical to at least 15 contiguous nucleotides of SEQ ID NO: 1, and a reverse primer comprising a sequence that is at least 85% complementary to at least 15 contiguous nucleotides of SEQ ID NO: 1; and
         3) a forward primer comprising a sequence that is at least 85% identical to at least 15 contiguous nucleotides of SEQ ID NO: 17, and a reverse primer comprising a sequence that is at least 85% identical to at least 15 contiguous nucleotides of SEQ ID NO: 18;
      ii) a set of primers that detects influenza A PA selected from:
         1) a forward and reverse primer for detecting a sequence of the influenza PA gene;
      2) a forward primer comprising a sequence that is at least 85% identical to at least 15 contiguous nucleotides of SEQ ID NO: 2, and a reverse primer comprising a sequence that is at least 85% complementary to at least 15 contiguous nucleotides of SEQ ID NO: 2; and
         3) a forward primer comprising a sequence that is at least 85% identical to at least 15 contiguous nucleotides of SEQ ID NO: 20, and a reverse primer comprising a sequence that is at least 85% identical to at least 15 contiguous nucleotides of SEQ ID NO: 21;
      iii) a set of primers that detects influenza A MP selected from:
         1) a forward and reverse primer for detecting a sequence of the influenza A MP gene;
         2) a forward primer comprising a sequence that is at least 85% identical to at least 15 contiguous nucleotides of SEQ ID NO: 3, and a reverse primer comprising a sequence that is at least 85% complementary to at least 15 contiguous nucleotides of SEQ ID NO: 3; and
         3) a forward primer comprising a sequence that is at least 85% identical to at least 15 contiguous nucleotides of SEQ ID NO: 23, and a reverse primer comprising a sequence that is at least 85% identical to at least 15 contiguous nucleotides of SEQ ID NO: 24;
      iv) a set of primers that detects avian influenza MP selected from:
         1) a forward and reverse primer for detecting a sequence of the avian influenza MP gene;
         2) a forward primer comprising a sequence that is at least 85% identical to at least 15 contiguous nucleotides of SEQ ID NO: 4, and a reverse primer comprising a sequence that is at least 85% complementary to at least 15 contiguous nucleotides of SEQ ID NO: 4; and
         3) a forward primer comprising a sequence that is at least 85% identical to at least 15 contiguous nucleotides of SEQ ID NO: 26, and a reverse primer comprising a sequence that is at least 85% identical to at least 15 contiguous nucleotides of SEQ ID NO: 27;
   b) wherein the set of primers that detects the presence or absence of influenza B comprises at least one of:
      i) a set of primers that detects influenza B MP selected from:
         1) a forward and reverse primer for detecting a sequence of the influenza B MP gene;
         2) a forward primer comprising a sequence that is at least 85% identical to at least 15 contiguous nucleotides of SEQ ID NO: 6, and a reverse primer comprising a sequence that is at least 85% complementary to at least 15 contiguous nucleotides of SEQ ID NO: 6; and
         3) a forward primer comprising a sequence that is at least 85% identical to at least 15 contiguous nucleotides of SEQ ID NO: 32, and a reverse primer comprising a sequence that is at least 85% identical to at least 15 contiguous nucleotides of SEQ ID NO: 33;
      ii) a set of primers that detects influenza B NS selected from:
         1) a forward and reverse primer for detecting a sequence of the influenza B NS gene;
         2) a forward primer comprising a sequence that is at least 85% identical to at least 15 contiguous nucleotides of SEQ ID NO: 7, and a reverse primer comprising a sequence that is at least 85% complementary to at least 15 contiguous nucleotides of SEQ ID NO: 7; and
         3) a forward primer comprising a sequence that is at least 85% identical to at least 15 contiguous nucleotides of SEQ ID NO: 35, and a reverse primer comprising a sequence that is at least 85% identical to at least 15 contiguous nucleotides of SEQ ID NO: 36;
   c) wherein the set of primers that detects the presence or absence of RSV comprises at least one of:
      i) a set of primers that detects RSV A selected from:
         1) at least one forward and at least one reverse primer for detecting a sequence of the RSV A gene; and
         2) at least one forward primer comprising a sequence that is at least 85% identical to at least 15 contiguous nucleotides of SEQ ID NO: 38 and/or SEQ ID NO: 67, and at least one reverse primer comprising a sequence that is at least 85% complementary to at least 15 contiguous nucleotides of SEQ ID NO: 39, SEQ ID NO: 68, and/or SEQ ID NO: 69; and
      ii) a set of primers that detects RSV B selected from:
         1) a forward and reverse primer for detecting a sequence of the RSV B gene; and
         2) a forward primer comprising a sequence that is at least 85% identical to at least 15 contiguous nucleotides of SEQ ID NO: 41, and a reverse primer comprising a sequence that is at least 85% identical to at least 15 contiguous nucleotides of SEQ ID NO: 42; and
   d) wherein the set of primers that detects the presence or absence of SARS-CoV-2 comprises at least one of:
      i) a set of primers that detects SARS-CoV-2 E selected from:
         1) a forward and reverse primer for detecting a sequence of the SARS-CoV-2 E gene;
         2) a forward primer comprising a sequence that is at least 85% identical to at least 15 contiguous nucleotides of SEQ ID NO: 44, and a reverse primer comprising a sequence that is at least 85% complementary to at least 15 contiguous nucleotides of SEQ ID NO: 44;
         3) a forward primer comprising a sequence that is at least 85% identical to at least 15 contiguous nucleotides of SEQ ID NO: 70, and a reverse primer comprising a sequence that is at least 85% identical to at least 15 contiguous nucleotides of SEQ ID NO: 71; and
         4) a forward primer comprising a sequence that is at least 85% identical to at least 15 contiguous nucleotides of SEQ ID NO: 48, and a reverse primer comprising a sequence that is at least 85% identical to at least 15 contiguous nucleotides of SEQ ID NO: 49;
      ii) a set of primers that detects SARS-CoV-2 N2 selected from:
         1) a forward and reverse primer for detecting a sequence of the SARS-CoV-2 N2 gene;
         2) a forward primer comprising a sequence that is at least 85% identical to at least 15 contiguous nucleotides of SEQ ID NO: 45, and a reverse primer comprising a sequence that is at least 85% complementary to at least 15 contiguous nucleotides of SEQ ID NO: 45; and
         3) a forward primer comprising a sequence that is at least 85% identical to at least 15 contiguous nucleotides of SEQ ID NO: 73 or SEQ ID NO: 51, and a reverse primer comprising a sequence that is at least 85% identical to at least 15 contiguous nucleotides of SEQ ID NO: 52;
      iii) a set of primers that detects SARS-CoV-2 RdRP selected from:
         1) a forward and at least one reverse primer for detecting a sequence of the SARS-CoV-2 RdRP gene;
         2) a forward primer comprising a sequence that is at least 85% identical to at least 15 contiguous nucleotides of SEQ ID NO: 76, and at least one reverse primer comprising a sequence that is at least 85% identical to at least 15 contiguous nucleotides of SEQ ID NO: 61 and/or SEQ ID NO: 78.
Embodiment 5. The method of embodiment 1 or embodiment 2,
   a) wherein the set of primers that detects the presence or absence of influenza A comprises at least one of:
      i) a set of primers that detects influenza A PB2 comprising a forward primer comprising a sequence that is at least 85% identical to at least 15 contiguous nucleotides of SEQ ID NO: 17, and a reverse primer comprising a sequence that is at least 85% identical to at least 15 contiguous nucleotides of SEQ ID NO: 18;
      ii) a set of primers that detects influenza A PA comprising a forward primer comprising a sequence that is at least 85% identical to at least 15 contiguous nucleotides of SEQ ID NO: 20, and a reverse primer comprising a sequence that is at least 85% identical to at least 15 contiguous nucleotides of SEQ ID NO: 21;
      iii) a set of primers that detects influenza A MP comprising a forward primer comprising a sequence that is at least 85% identical to at least 15 contiguous nucleotides of SEQ ID NO: 23, and a reverse primer comprising a sequence that is at least 85% identical to at least 15 contiguous nucleotides of SEQ ID NO: 24; and
      iv) a set of primers that detects avian influenza MP comprising a forward primer comprising a sequence that is at least 85% identical to at least 15 contiguous nucleotides of SEQ ID NO: 26, and a reverse primer comprising a sequence that is at least 85% identical to at least 15 contiguous nucleotides of SEQ ID NO: 27;
   b) wherein the set of primers that detects the presence or absence of influenza B comprises at least one of:
      i) a set of primers that detects influenza B MP comprising a forward primer comprising a sequence that is at least 85% identical to at least 15 contiguous nucleotides of SEQ ID NO: 32, and a reverse primer comprising a sequence that is at least 85% identical to at least 15 contiguous nucleotides of SEQ ID NO: 33; and
      ii) a set of primers that detects influenza B NS comprising a forward primer comprising a sequence that is at least 85% identical to at least 15 contiguous nucleotides of SEQ ID NO: 35, and a reverse primer comprising a sequence that is at least 85% identical to at least 15 contiguous nucleotides of SEQ ID NO: 36;
   c) wherein the set of primers that detects the presence or absence of RSV comprises at least one of:
      i) a set of primers that detects RSV A comprising at least one forward primer comprising a sequence that is at least 85% identical to at least 15 contiguous nucleotides of SEQ ID NO: 38 and/or SEQ ID NO: 67, and at least one reverse primer comprising a sequence that is at least 85% complementary to at least 15 contiguous nucleotides of SEQ ID NO: 39, SEQ ID NO: 68, and/or SEQ ID NO: 69; and
      ii) a set of primers that detects RSV B comprising a forward primer comprising a sequence that is at least 85% identical to at least 15 contiguous nucleotides of SEQ ID NO: 41, and a reverse primer comprising a sequence that is at least 85% identical to at least 15 contiguous nucleotides of SEQ ID NO: 42; and
   d) wherein the set of primers that detects the presence or absence of SARS-CoV-2 comprises at least one of:
      i) a set of primers that detects SARS-CoV-2 E comprising a forward primer comprising a sequence that is at least 85% identical to at least 15 contiguous nucleotides of SEQ ID NO: 70, and a reverse primer comprising a sequence that is at least 85% identical to at least 15 contiguous nucleotides of SEQ ID NO: 71;
      ii) a set of primers that detects SARS-CoV-2 N2 comprising a forward primer comprising a sequence that is at least 85% identical to at least 15 contiguous nucleotides of SEQ ID NO: 73, and a reverse primer comprising a sequence that is at least 85% identical to at least 15 contiguous nucleotides of SEQ ID NO: 52; and
      iii) a set of primers that detects SARS-CoV-2 RdRP comprising a forward primer comprising a sequence that is at least 85% identical to at least 15 contiguous nucleotides of SEQ ID NO: 76, and at least one reverse primer comprising a sequence that is at least 85% identical to at least 15 contiguous nucleotides of SEQ ID NO: 61 and/or SEQ ID NO: 78.
Embodiment 6. The method of any one of the preceding embodiments wherein:
   a) the influenza A PA amplicon comprises a sequence that is at least 85% identical to SEQ ID NO: 9;
   b) the influenza A PB2 amplicon comprises a sequence that is at least 85% identical to SEQ ID NO: 8;
   c) the influenza A MP amplicon comprises a sequence that is at least 85% identical to SEQ ID NO: 10;
   d) the avian influenza MP amplicon comprises a sequence that is at least 85% identical to SEQ ID NO: 11;
   e) the influenza B MP amplicon comprises a sequence that is at least 85% identical to SEQ ID NO: 13;
   f) the influenza B NS amplicon comprises a sequence that is at least 85% identical to SEQ ID NO: 14;
   g) the RSV A amplicon comprises a sequence that is at least 85% identical to SEQ ID NO: 15;
   h) the RSV B amplicon comprises a sequence that is at least 85% identical to SEQ ID NO: 16;
   i) the SARS-CoV-2 E amplicon comprises a sequence that is at least 85% identical to SEQ ID NO: 46 or SEQ ID NO: 79;
   j) the SARS-CoV-2 N2 amplicon comprises a sequence that is at least 85% identical to SEQ ID NO: 47; and/or
   k) the SARS-CoV-2 RdRP amplicon comprises a sequence that is at least 85% identical to SEQ ID NO: 80 and/or SEQ ID NO: 81.
Embodiment 7. The method of any one of the preceding embodiments, wherein the method further comprises contacting the amplicons with at least one probe selected from an influenza A PA probe, an influenza A PB2 probe, an influenza A MP probe, an avian influenza MP probe, an influenza B MP probe, an influenza B NS probe, an RSV A probe, an RSV B probe, a SARS-CoV-2 E probe, a SARS-CoV-2 N2 probe, and a SARS-CoV-2 RdRP probe.
Embodiment 8. The method of embodiment 7, wherein
   a) the influenza A PA probe comprises a sequence that is at least 85% identical or complementary to at least 15 contiguous nucleotides of SEQ ID NO: 22;
   b) the influenza A PB2 probe comprises a sequence that is at least 85% identical or complementary to at least 15 contiguous nucleotides of SEQ ID NO: 19;
   c) the influenza A MP probe comprises a sequence that is at least 85% identical or complementary to at least 15 contiguous nucleotides of SEQ ID NO: 25;
   d) the avian influenza MP probe comprises a sequence that is at least 85% identical or complementary to at least 15 contiguous nucleotides of SEQ ID NO: 28;
   e) the influenza B MP probe comprises a sequence that is at least 85% identical or complementary to at least 15 contiguous nucleotides of SEQ ID NO: 34;
   f) the influenza B NS probe comprises a sequence that is at least 85% identical or complementary to at least 15 contiguous nucleotides of SEQ ID NO: 37;
   g) the RSV A probe comprises a sequence that is at least 85% identical or complementary to at least 15 contiguous nucleotides of SEQ ID NO: 40;
   h) the RSV B probe comprises a sequence that is at least 85% identical or complementary to at least 15 contiguous nucleotides of SEQ ID NO: 43;
   i) the SARS-CoV-2 E probe comprises a sequence that is at least 85% identical or complementary to at least 15 contiguous nucleotides of SEQ ID NO: 72, SEQ ID NO: 50 or SEQ ID NO: 56;
   j) the SARS-CoV-2 N2 probe comprises a sequence that is at least 85% identical or complementary to at least 15 contiguous nucleotides of SEQ ID NO: 74, SEQ ID NO: 75, and/or SEQ ID NO: 53; and/or
   k) the SARS-CoV-2 RdRP probe comprises a sequence that is at least 85% identical or complementary to at least 15 contiguous nucleotides of SEQ ID NO: 77.
Embodiment 9. The method of any one of the preceding embodiments, wherein the method further comprises detecting an exogenous control.
Embodiment 10. The method of embodiment 9, wherein the exogenous control is a sample processing control.
Embodiment 11. The method of any one of embodiments 9 to 10, wherein the exogenous control is an RNA control packaged in a bacteriophage protective coat.
Embodiment 12. The method of any one of embodiments 9 to 11, wherein the method comprises contacting nucleic acids from the sample with a control primer pair for detecting an exogenous control.
Embodiment 13. The method of any one of embodiments 9 to 12, wherein the method comprises forming an exogenous control amplicon and contacting the exogenous control amplicon with a control probe capable of selectively hybridizing with the exogenous control amplicon.
Embodiment 14. The method of any one of embodiments 7 to 13, wherein each probe comprises a detectable label.
Embodiment 15. The method of any one of embodiments 7 to 14, wherein each probe comprises a fluorescent dye and a quencher molecule.
Embodiment 16. The method of any one of embodiments 7 to 5, wherein the probes for detecting different target molecules comprise detectable labels that are detectably different.
Embodiment 17. The method of any one of embodiments 7 to 16, wherein the probes for detecting different target molecules comprise detectable labels that are not detectably different.
Embodiment 18. The method of any one of the preceding embodiments, wherein the PCR is quantitative PCR.
Embodiment 19. The method of any one of the preceding embodiments, wherein the PCR reaction takes less than 2 hours from an initial denaturation step through a final extension step.
Embodiment 20. The method of any one of the preceding embodiments, wherein the PCR reaction takes less than 1 hour from an initial denaturation step through a final extension step.
Embodiment 21. The method of any one of the preceding embodiments, wherein the subject has one or more symptoms of influenza, RSV, and/or COVID-19.
Embodiment 22. The method of any one of the preceding embodiments, wherein the subject has one or more symptoms selected from fever, chills, cough, shortness of breath or difficulty breathing, sore throat, runny nose, nasal congestion, muscle or body ache, headache, fatigue, new loss of taste or smell, nausea or vomiting, and diarrhea.
Embodiment 23. The method of any one of the preceding embodiments, wherein the sample is selected from a nasopharyngeal swab sample, an oropharyngeal sample, a nasal aspirate sample, a nasal or mid-turbinate swab, a nasal aspirate sample, a nasal wash sample, a throat swab sample, a bronchoalveolar lavage sample, a bronchial aspirate sample, a bronchial wash sample, an endotracheal aspirate, an endotracheal wash sample, a tracheal aspirate, a nasal secretion sample, a mucus sample, a sputum sample, a lung tissue samples, a urine sample, a saliva sample and a fecal sample.
Embodiment 24. A composition comprising sets of primers that detect the presence of an influenza A gene, an influenza B gene, a RSV gene, and a SARS-CoV-2 gene,
   a) wherein the set of primers that detects the presence of influenza A comprises at least one of:
      i) a set of primers that detects influenza A PB2 selected from:
         1) a forward and reverse primer for detecting a sequence of the influenza PB2 gene;
         2) a forward primer comprising a sequence that is at least 85% identical to at least 15 contiguous nucleotides of SEQ ID NO: 1, and a reverse primer comprising a sequence that is at least 85% complementary to at least 15 contiguous nucleotides of SEQ ID NO: 1; and
         3) a forward primer comprising a sequence that is at least 85% identical to at least 15 contiguous nucleotides of SEQ ID NO: 17, and a reverse primer comprising a sequence that is at least 85% identical to at least 15 contiguous nucleotides of SEQ ID NO: 18;
      ii) a set of primers that detects influenza A PA selected from:
         1) a forward and reverse primer for detecting a sequence of the influenza PA gene;
         2) a forward primer comprising a sequence that is at least 85% identical to at least 15 contiguous nucleotides of SEQ ID NO: 2, and a reverse primer comprising a sequence that is at least 85% complementary to at least 15 contiguous nucleotides of SEQ ID NO: 2; and
         3) a forward primer comprising a sequence that is at least 85% identical to at least 15 contiguous nucleotides of SEQ ID NO: 20, and a reverse primer comprising a sequence that is at least 85% identical to at least 15 contiguous nucleotides of SEQ ID NO: 21;
      iii) a set of primers that detects influenza A MP selected from:
         1) a forward and reverse primer for detecting a sequence of the influenza A MP gene;
         2) a forward primer comprising a sequence that is at least 85% identical to at least 15 contiguous nucleotides of SEQ ID NO: 3, and a reverse primer comprising a sequence that is at least 85% complementary to at least 15 contiguous nucleotides of SEQ ID NO: 3; and
         3) a forward primer comprising a sequence that is at least 85% identical to at least 15 contiguous nucleotides of SEQ ID NO: 23, and a reverse primer comprising a sequence that is at least 85% identical to at least 15 contiguous nucleotides of SEQ ID NO: 24;
      iv) a set of primers that detects avian influenza MP selected from:
         1) a forward and reverse primer for detecting a sequence of the avian influenza MP gene;
         2) a forward primer comprising a sequence that is at least 85% identical to at least 15 contiguous nucleotides of SEQ ID NO: 4, and a reverse primer comprising a sequence that is at least 85% complementary to at least 15 contiguous nucleotides of SEQ ID NO: 4; and
         3) a forward primer comprising a sequence that is at least 85% identical to at least 15 contiguous nucleotides of SEQ ID NO: 26, and a reverse primer comprising a sequence that is at least 85% identical to at least 15 contiguous nucleotides of SEQ ID NO: 27;
   b) wherein the set of primers that detects the presence of influenza B comprises at least one of:
      i) a set of primers that detects influenza B MP selected from:
         1) a forward and reverse primer for detecting a sequence of the influenza B MP gene;
         2) a forward primer comprising a sequence that is at least 85% identical to at least 15 contiguous nucleotides of SEQ ID NO: 6, and a reverse primer comprising a sequence that is at least 85% complementary to at least 15 contiguous nucleotides of SEQ ID NO: 6; and
         3) a forward primer comprising a sequence that is at least 85% identical to at least 15 contiguous nucleotides of SEQ ID NO: 32, and a reverse primer comprising a sequence that is at least 85% identical to at least 15 contiguous nucleotides of SEQ ID NO: 33;
      ii) a set of primers that detects influenza B NS selected from:
         1) a forward and reverse primer for detecting a sequence of the influenza B NS gene;
         2) a forward primer comprising a sequence that is at least 85% identical to at least 15 contiguous nucleotides of SEQ ID NO: 7, and a reverse primer comprising a sequence that is at least 85% complementary to at least 15 contiguous nucleotides of SEQ ID NO: 7; and
         3) a forward primer comprising a sequence that is at least 85% identical to at least 15 contiguous nucleotides of SEQ ID NO: 35, and a reverse primer comprising a sequence that is at least 85% identical to at least 15 contiguous nucleotides of SEQ ID NO: 36;
   c) wherein the set of primers that detects the presence of RSV comprises at least one of:
      i) a set of primers that detects RSV A selected from:
         1) a forward and reverse primer for detecting a sequence of the RSV A gene; and
         2) a forward primer comprising a sequence that is at least 85% identical to at least 15 contiguous nucleotides of SEQ ID NO: 38, and a reverse primer comprising a sequence that is at least 85% complementary to at least 15 contiguous nucleotides of SEQ ID NO: 39; and
      ii) a set of primers that detects RSV B selected from:
         1) a forward and reverse primer for detecting a sequence of the RSV B gene; and
         2) a forward primer comprising a sequence that is at least 85% identical to at least 15 contiguous nucleotides of SEQ ID NO: 41, and a reverse primer comprising a sequence that is at least 85% identical to at least 15 contiguous nucleotides of SEQ ID NO: 42; and
   d) wherein the set of primers that detects the presence of SARS-CoV-2 comprises at least one of:
      i) a set of primers that detects SARS-CoV-2 E selected from:
         1) a forward and reverse primer for detecting a sequence of the SARS-CoV-2 E gene;
         2) a forward primer comprising a sequence that is at least 85% identical to at least 15 contiguous nucleotides of SEQ ID NO: 44, and a reverse primer comprising a sequence that is at least 85% complementary to at least 15 contiguous nucleotides of SEQ ID NO: 44; and
         3) a forward primer comprising a sequence that is at least 85% identical to at least 15 contiguous nucleotides of SEQ ID NO: 48, and a reverse primer comprising a sequence that is at least 85% identical to at least 15 contiguous nucleotides of SEQ ID NO: 49; and
      ii) a set of primers that detects SARS-CoV-2 N2 selected from:
         1) a forward and reverse primer for detecting a sequence of the SARS-CoV-2 N2 gene;
         2) a forward primer comprising a sequence that is at least 85% identical to at least 15 contiguous nucleotides of SEQ ID NO: 45, and a reverse primer comprising a sequence that is at least 85% complementary to at least 15 contiguous nucleotides of SEQ ID NO: 45; and
         3) a forward primer comprising a sequence that is at least 85% identical to at least 15 contiguous nucleotides of SEQ ID NO: 51, and a reverse primer comprising a sequence that is at least 85% identical to at least 15 contiguous nucleotides of SEQ ID NO: 52.
Embodiment 25. A composition comprising sets of primers that detect the presence of an influenza A gene, an influenza B gene, a RSV gene, and a SARS-CoV-2 gene,
   a) wherein the set of primers that detects the presence or absence of influenza A comprises at least one of:
      i) a set of primers that detects influenza A PB2 selected from:
         1) a forward and reverse primer for detecting a sequence of the influenza PB2 gene;
         2) a forward primer comprising a sequence that is at least 85% identical to at least 15 contiguous nucleotides of SEQ ID NO: 1, and a reverse primer comprising a sequence that is at least 85% complementary to at least 15 contiguous nucleotides of SEQ ID NO: 1; and
         3) a forward primer comprising a sequence that is at least 85% identical to at least 15 contiguous nucleotides of SEQ ID NO: 17, and a reverse primer comprising a sequence that is at least 85% identical to at least 15 contiguous nucleotides of SEQ ID NO: 18;
      ii) a set of primers that detects influenza A PA selected from:
         1) a forward and reverse primer for detecting a sequence of the influenza PA gene;
         2) a forward primer comprising a sequence that is at least 85% identical to at least 15 contiguous nucleotides of SEQ ID NO: 2, and a reverse primer comprising a sequence that is at least 85% complementary to at least 15 contiguous nucleotides of SEQ ID NO: 2; and
         3) a forward primer comprising a sequence that is at least 85% identical to at least 15 contiguous nucleotides of SEQ ID NO: 20, and a reverse primer comprising a sequence that is at least 85% identical to at least 15 contiguous nucleotides of SEQ ID NO: 21;
      iii) a set of primers that detects influenza A MP selected from:
         1) a forward and reverse primer for detecting a sequence of the influenza A MP gene;
         2) a forward primer comprising a sequence that is at least 85% identical to at least 15 contiguous nucleotides of SEQ ID NO: 3, and a reverse primer comprising a sequence that is at least 85% complementary to at least 15 contiguous nucleotides of SEQ ID NO: 3; and
         3) a forward primer comprising a sequence that is at least 85% identical to at least 15 contiguous nucleotides of SEQ ID NO: 23, and a reverse primer comprising a sequence that is at least 85% identical to at least 15 contiguous nucleotides of SEQ ID NO: 24;
      iv) a set of primers that detects avian influenza MP selected from:
         1) a forward and reverse primer for detecting a sequence of the avian influenza MP gene;
         2) a forward primer comprising a sequence that is at least 85% identical to at least 15 contiguous nucleotides of SEQ ID NO: 4, and a reverse primer comprising a sequence that is at least 85% complementary to at least 15 contiguous nucleotides of SEQ ID NO: 4; and
         3) a forward primer comprising a sequence that is at least 85% identical to at least 15 contiguous nucleotides of SEQ ID NO: 26, and a reverse primer comprising a sequence that is at least 85% identical to at least 15 contiguous nucleotides of SEQ ID NO: 27;
   b) wherein the set of primers that detects the presence or absence of influenza B comprises at least one of:
      i) a set of primers that detects influenza B MP selected from:
         1) a forward and reverse primer for detecting a sequence of the influenza B MP gene;
         2) a forward primer comprising a sequence that is at least 85% identical to at least 15 contiguous nucleotides of SEQ ID NO: 6, and a reverse primer comprising a sequence that is at least 85% complementary to at least 15 contiguous nucleotides of SEQ ID NO: 6; and
         3) a forward primer comprising a sequence that is at least 85% identical to at least 15 contiguous nucleotides of SEQ ID NO: 32, and a reverse primer comprising a sequence that is at least 85% identical to at least 15 contiguous nucleotides of SEQ ID NO: 33;
      ii) a set of primers that detects influenza B NS selected from:
         1) a forward and reverse primer for detecting a sequence of the influenza B NS gene;
         2) a forward primer comprising a sequence that is at least 85% identical to at least 15 contiguous nucleotides of SEQ ID NO: 7, and a reverse primer comprising a sequence that is at least 85% complementary to at least 15 contiguous nucleotides of SEQ ID NO: 7; and
         3) a forward primer comprising a sequence that is at least 85% identical to at least 15 contiguous nucleotides of SEQ ID NO: 35, and a reverse primer comprising a sequence that is at least 85% identical to at least 15 contiguous nucleotides of SEQ ID NO: 36;
   c) wherein the set of primers that detects the presence or absence of RSV comprises at least one of:
      i) a set of primers that detects RSV A selected from:
         1) at least one forward and at least one reverse primer for detecting a sequence of the RSV A gene; and
         2) at least one forward primer comprising a sequence that is at least 85% identical to at least 15 contiguous nucleotides of SEQ ID NO: 38 and/or SEQ ID NO: 67, and at least one reverse primer comprising a sequence that is at least 85% complementary to at least 15 contiguous nucleotides of SEQ ID NO: 39, SEQ ID NO: 68, and/or SEQ ID NO: 69; and
      ii) a set of primers that detects RSV B selected from:
         1) a forward and reverse primer for detecting a sequence of the RSV B gene; and
         2) a forward primer comprising a sequence that is at least 85% identical to at least 15 contiguous nucleotides of SEQ ID NO: 41, and a reverse primer comprising a sequence that is at least 85% identical to at least 15 contiguous nucleotides of SEQ ID NO: 42; and
   d) wherein the set of primers that detects the presence or absence of SARS-CoV-2 comprises at least one of:
      i) a set of primers that detects SARS-CoV-2 E selected from:
         1) a forward and reverse primer for detecting a sequence of the SARS-CoV-2 E gene;
         2) a forward primer comprising a sequence that is at least 85% identical to at least 15 contiguous nucleotides of SEQ ID NO: 44, and a reverse primer comprising a sequence that is at least 85% complementary to at least 15 contiguous nucleotides of SEQ ID NO: 44;
         3) a forward primer comprising a sequence that is at least 85% identical to at least 15 contiguous nucleotides of SEQ ID NO: 70, and a reverse primer comprising a sequence that is at least 85% identical to at least 15 contiguous nucleotides of SEQ ID NO: 71; and
         4) a forward primer comprising a sequence that is at least 85% identical to at least 15 contiguous nucleotides of SEQ ID NO: 48, and a reverse primer comprising a sequence that is at least 85% identical to at least 15 contiguous nucleotides of SEQ ID NO: 49;
      ii) a set of primers that detects SARS-CoV-2 N2 selected from:
         1) a forward and reverse primer for detecting a sequence of the SARS-CoV-2 N2 gene;
         2) a forward primer comprising a sequence that is at least 85% identical to at least 15 contiguous nucleotides of SEQ ID NO: 45, and a reverse primer comprising a sequence that is at least 85% complementary to at least 15 contiguous nucleotides of SEQ ID NO: 45; and
         3) a forward primer comprising a sequence that is at least 85% identical to at least 15 contiguous nucleotides of SEQ ID NO: 73 or SEQ ID NO: 51, and a reverse primer comprising a sequence that is at least 85% identical to at least 15 contiguous nucleotides of SEQ ID NO: 52
      iii) a set of primers that detects SARS-CoV-2 RdRP selected from:
         1) a forward and at least one reverse primer for detecting a sequence of the SARS-CoV-2 RdRP gene;
         2) a forward primer comprising a sequence that is at least 85% identical to at least 15 contiguous nucleotides of SEQ ID NO: 76, and at least one reverse primer comprising a sequence that is at least 85% identical to at least 15 contiguous nucleotides of SEQ ID NO: 61 and/or SEQ ID NO: 78.
Embodiment 26. A composition comprising sets of primers that detect the presence of an influenza A gene, an influenza B gene, a RSV gene, and a SARS-CoV-2 gene,
   a) wherein the set of primers that detects the presence or absence of influenza A comprises at least one of:
      i) a set of primers that detects influenza A PB2 comprising a forward primer comprising a sequence that is at least 85% identical to at least 15 contiguous nucleotides of SEQ ID NO: 17, and a reverse primer comprising a sequence that is at least 85% identical to at least 15 contiguous nucleotides of SEQ ID NO: 18;
      ii) a set of primers that detects influenza A PA comprising a forward primer comprising a sequence that is at least 85% identical to at least 15 contiguous nucleotides of SEQ ID NO: 20, and a reverse primer comprising a sequence that is at least 85% identical to at least 15 contiguous nucleotides of SEQ ID NO: 21;
      iii) a set of primers that detects influenza A MP comprising a forward primer comprising a sequence that is at least 85% identical to at least 15 contiguous nucleotides of SEQ ID NO: 23, and a reverse primer comprising a sequence that is at least 85% identical to at least 15 contiguous nucleotides of SEQ ID NO: 24; and
      iv) a set of primers that detects avian influenza MP comprising a forward primer comprising a sequence that is at least 85% identical to at least 15 contiguous nucleotides of SEQ ID NO: 26, and a reverse primer comprising a sequence that is at least 85% identical to at least 15 contiguous nucleotides of SEQ ID NO: 27;
   b) wherein the set of primers that detects the presence or absence of influenza B comprises at least one of:
      i) a set of primers that detects influenza B MP comprising a forward primer comprising a sequence that is at least 85% identical to at least 15 contiguous nucleotides of SEQ ID NO: 32, and a reverse primer comprising a sequence that is at least 85% identical to at least 15 contiguous nucleotides of SEQ ID NO: 33; and
      ii) a set of primers that detects influenza B NS comprising a forward primer comprising a sequence that is at least 85% identical to at least 15 contiguous nucleotides of SEQ ID NO: 35, and a reverse primer comprising a sequence that is at least 85% identical to at least 15 contiguous nucleotides of SEQ ID NO: 36;
   c) wherein the set of primers that detects the presence or absence of RSV comprises at least one of:
      i) a set of primers that detects RSV A comprising at least one forward primer comprising a sequence that is at least 85% identical to at least 15 contiguous nucleotides of SEQ ID NO: 38 and/or SEQ ID NO: 67, and at least one reverse primer comprising a sequence that is at least 85% complementary to at least 15 contiguous nucleotides of SEQ ID NO: 39, SEQ ID NO: 68, and/or SEQ ID NO: 69; and
      ii) a set of primers that detects RSV B comprising a forward primer comprising a sequence that is at least 85% identical to at least 15 contiguous nucleotides of SEQ ID NO: 41, and a reverse primer comprising a sequence that is at least 85% identical to at least 15 contiguous nucleotides of SEQ ID NO: 42; and
   d) wherein the set of primers that detects the presence or absence of SARS-CoV-2 comprises at least one of:
      i) a set of primers that detects SARS-CoV-2 E comprising a forward primer comprising a sequence that is at least 85% identical to at least 15 contiguous nucleotides of SEQ ID NO: 70, and a reverse primer comprising a sequence that is at least 85% identical to at least 15 contiguous nucleotides of SEQ ID NO: 71;
      ii) a set of primers that detects SARS-CoV-2 N2 comprising a forward primer comprising a sequence that is at least 85% identical to at least 15 contiguous nucleotides of SEQ ID NO: 73, and a reverse primer comprising a sequence that is at least 85% identical to at least 15 contiguous nucleotides of SEQ ID NO: 52; and
      iii) a set of primers that detects SARS-CoV-2 RdRP comprising a forward primer comprising a sequence that is at least 85% identical to at least 15 contiguous nucleotides of SEQ ID NO: 76, and at least one reverse primer comprising a sequence that is at least 85% identical to at least 15 contiguous nucleotides of SEQ ID NO: 61 and/or SEQ ID NO: 78.
Embodiment 27. The composition of any one of embodiments 24 to 26, further comprising a primer pair for detecting an exogenous control.
Embodiment 28. The composition of embodiment 27, wherein the exogenous control is a sample processing control.
Embodiment 29. The method of any one of embodiments 24 to 27, wherein the exogenous control is an RNA control packaged in a bacteriophage protective coat.
Embodiment 30. The composition of any one of embodiments 24 to 29, further comprising at least one probe selected from an influenza A PA probe, an influenza A PB2 probe, an influenza A MP probe, an avian influenza MP probe, an influenza B MP probe, an influenza B NS probe, an RSV A probe, an RSV B probe, a SARS-CoV-2 E probe, a SARS-CoV-2 N2 probe, and a SARS-CoV-2 RdRP probe.
Embodiment 31. The composition of embodiment 30, wherein
   a) the influenza A PA probe comprises a sequence that is at least 85% identical or complementary to at least 15 contiguous nucleotides of SEQ ID NO: 22;
   b) the influenza A PB2 probe comprises a sequence that is at least 85% identical or complementary to at least 15 contiguous nucleotides of SEQ ID NO: 19;
   c) the influenza A MP probe comprises a sequence that is at least 85% identical or complementary to at least 15 contiguous nucleotides of SEQ ID NO: 25;
   d) the avian influenza MP probe comprises a sequence that is at least 85% identical or complementary to at least 15 contiguous nucleotides of SEQ ID NO: 28;
   e) the influenza B MP probe comprises a sequence that is at least 85% identical or complementary to at least 15 contiguous nucleotides of SEQ ID NO: 34;
   f) the influenza B NS probe comprises a sequence that is at least 85% identical or complementary to at least 15 contiguous nucleotides of SEQ ID NO: 37;
   g) the RSV A probe comprises a sequence that is at least 85% identical or complementary to at least 15 contiguous nucleotides of SEQ ID NO: 40;
   h) the RSV B probe comprises a sequence that is at least 85% identical or complementary to at least 15 contiguous nucleotides of SEQ ID NO: 43;
   i) the SARS-CoV-2 E probe comprises a sequence that is at least 85% identical or complementary to at least 15 contiguous nucleotides of SEQ ID NO: 72, SEQ ID NO: 50 or SEQ ID NO: 56;
   j) the SARS-CoV-2 N2 probe comprises a sequence that is at least 85% identical or complementary to at least 15 contiguous nucleotides of SEQ ID NO: 74, SEQ ID NO: 75, and/or SEQ ID NO: 53; and/or
   k) the SARS-CoV-2 RdRP probe comprises a sequence that is at least 85% identical or complementary to at least 15 contiguous nucleotides of SEQ ID NO: 77.
Embodiment 32. The composition of any one of embodiments 24 to 31, further comprising a probe for detecting an exogenous control.
Embodiment 33. The composition of any one of embodiments 24 to 32, wherein each probe comprises a detectable label.
Embodiment 34. The composition of embodiment 33, wherein each probe comprises a fluorescent dye and a quencher molecule.
Embodiment 35. The composition of any one of embodiments 24 to 34, wherein the composition comprises sets of primers that are lyophilized.
Embodiment 36. The composition of any one of embodiments 24 to 34, wherein the composition comprises sets of primers that are in solution.
Embodiment 37. The composition of any one of embodiments 24 to 36, wherein the composition comprises nucleic acids from a sample from a subject being tested for the presence of absence of influenza, RSV, and/or COVID-19.
Embodiment 38. The composition of embodiment 37, wherein the sample is selected from a nasopharyngeal swab sample, an oropharyngeal sample, a nasal aspirate sample, a nasal or mid-turbinate swab, a nasal aspirate sample, a nasal wash sample, a throat swab sample, a bronchoalveolar lavage sample, a bronchial aspirate sample, a bronchial wash sample, an endotracheal aspirate, an endotracheal wash sample, a tracheal aspirate, a nasal secretion sample, a mucus sample, a sputum sample, a lung tissue samples, a urine sample, a saliva sample and a fecal sample.
Embodiment 39. A kit comprising a composition of any one of embodiments 24 to 38.
Embodiment 40. The kit of embodiment 39, wherein the kit further comprises an exogenous control.
Embodiment 41. The kit of embodiment 40, wherein the exogenous control is an RNA control packaged in a bacteriophage protective coat.
Embodiment 42. The kit of any one of embodiments 39 to 41, wherein the kit comprises dNTPs and/or a thermostable polymerase.
Embodiment 43. The kit of any one of embodiments 39 to 42, wherein the kit comprises a reverse transcriptase.
Embodiment 44. The method of any one of embodiments 1 to 23, wherein the method comprises detecting the presence or absence of at least one influenza A gene, at least one influenza B gene, at least one RSV gene, at least one SARS-CoV-2 gene, and an exogenous control in a single multiplex reaction.
Embodiment 45. The method of embodiment 44, wherein the cycle threshold (Ct) of the reaction is less than 40 cycles.
Embodiment 46. A method of detecting the presence or absence of SARS-CoV-2 in a biological sample from a subject and/or determining whether a subject has COVID-19, comprising:
   a) contacting a biological sample from the subject with sets of primers that detect a SARS-CoV-2 E and/or N2 gene;
   b) conducting one or more polymerase chain reaction (PCR); and
   c) detecting an amplicon that is produced by the PCR; wherein the set of primers that detects the presence or absence of SARS-CoV-2 E and/or N2 genes comprises at least one of:
   a) a set of primers that detects SARS-CoV-2 E selected from:
      i) a forward and reverse primer for detecting a sequence of the SARS-CoV-2 E gene;
      ii) a forward primer comprising a sequence that is at least 85% identical to at least 15 contiguous nucleotides of SEQ ID NO: 44, and a reverse primer comprising a sequence that is at least 85% complementary to at least 15 contiguous nucleotides of SEQ ID NO: 44; and
      iii) a forward primer comprising a sequence that is at least 85% identical to at least 15 contiguous nucleotides of SEQ ID NO: 48, and a reverse primer comprising a sequence that is at least 85% identical to at least 15 contiguous nucleotides of SEQ ID NO: 49; and
   b) a set of primers that detects SARS-CoV-2 N2 selected from:
      i) a forward and reverse primer for detecting a sequence of the SARS-CoV-2 N2 gene;
      ii) a forward primer comprising a sequence that is at least 85% identical to at least 15 contiguous nucleotides of SEQ ID NO: 45, and a reverse primer comprising a sequence that is at least 85% complementary to at least 15 contiguous nucleotides of SEQ ID NO: 45; and
      iii) a forward primer comprising a sequence that is at least 85% identical to at least 15 contiguous nucleotides of SEQ ID NO: 51, and a reverse primer comprising a sequence that is at least 85% identical to at least 15 contiguous nucleotides of SEQ ID NO: 52.
Embodiment 47. A method of detecting the presence or absence of SARS-CoV-2 in a biological sample from a subject and/or determining whether a subject has COVID-19, comprising:
   a) contacting a biological sample from the subject with sets of primers that detect a SARS-CoV-2 E, SARS-CoV-2 N2 gene, and/or SARS-CoV-2 RdRP gene;
   b) conducting one or more polymerase chain reaction (PCR); and
   c) detecting an amplicon that is produced by the PCR; wherein the set of primers that detects the presence or absence of SARS-CoV-2 E, SARS-CoV-2 N2 gene, and/or SARS-CoV-2 RdRP genes comprises at least one of:
   a) a set of primers that detects SARS-CoV-2 E selected from:
      i) a forward and reverse primer for detecting a sequence of the SARS-CoV-2 E gene;
      ii) a forward primer comprising a sequence that is at least 85% identical to at least 15 contiguous nucleotides of SEQ ID NO: 44, and a reverse primer comprising a sequence that is at least 85% complementary to at least 15 contiguous nucleotides of SEQ ID NO: 44; and
      iii) a forward primer comprising a sequence that is at least 85% identical to at least 15 contiguous nucleotides of SEQ ID NO: 70, and a reverse primer comprising a sequence that is at least 85% identical to at least 15 contiguous nucleotides of SEQ ID NO: 71; and
      iv) a forward primer comprising a sequence that is at least 85% identical to at least 15 contiguous nucleotides of SEQ ID NO: 48, and a reverse primer comprising a sequence that is at least 85% identical to at least 15 contiguous nucleotides of SEQ ID NO: 49;
   b) a set of primers that detects SARS-CoV-2 N2 selected from:
      i) a forward and reverse primer for detecting a sequence of the SARS-CoV-2 N2 gene;
      ii) a forward primer comprising a sequence that is at least 85% identical to at least 15 contiguous nucleotides of SEQ ID NO: 45, and a reverse primer comprising a sequence that is at least 85% complementary to at least 15 contiguous nucleotides of SEQ ID NO: 45; and
      iii) a forward primer comprising a sequence that is at least 85% identical to at least 15 contiguous nucleotides of SEQ ID NO: 73 or SEQ ID NO: 51, and a reverse primer comprising a sequence that is at least 85% identical to at least 15 contiguous nucleotides of SEQ ID NO: 52; and
   d) a set of primers that detects SARS-CoV-2 RdRP selected from:
      i) a forward and at least one reverse primer for detecting a sequence of the SARS-CoV-2 RdRP gene;
      ii) a forward primer comprising a sequence that is at least 85% identical to at least 15 contiguous nucleotides of SEQ ID NO: 76, and at least one reverse primer comprising a sequence that is at least 85% identical to at least 15 contiguous nucleotides of SEQ ID NO: 61 and/or SEQ ID NO: 78.
Embodiment 48. The method of embodiment 47, wherein
   a) the set of primers that detects SARS-CoV-2 E comprises a forward primer comprising a sequence that is at least 85% identical to at least 15 contiguous nucleotides of SEQ ID NO: 70, and a reverse primer comprising a sequence that is at least 85% identical to at least 15 contiguous nucleotides of SEQ ID NO: 71;
   b) the set of primers that detects SARS-CoV-2 N2 comprises a forward primer comprising a sequence that is at least 85% identical to at least 15 contiguous nucleotides of SEQ ID NO: 73, and a reverse primer comprising a sequence that is at least 85% identical to at least 15 contiguous nucleotides of SEQ ID NO: 52; and
   c) the set of primers that detects SARS-CoV-2 RdRP comprises a forward primer comprising a sequence that is at least 85% identical to at least 15 contiguous nucleotides of SEQ ID NO: 76, and at least one reverse primer comprising a sequence that is at least 85% identical to at least 15 contiguous nucleotides of SEQ ID NO: 61 and/or SEQ ID NO: 78.
Embodiment 49. The method of embodiment 47 or embodiment 48, wherein
   a) the SARS-CoV-2 E amplicon comprises a sequence that is at least 85% identical to SEQ ID NO: 79;
   b) the SARS-CoV-2 N2 amplicon comprises a sequence that is at least 85% identical to SEQ ID NO: 47; and/or
   c) the SARS-CoV-2 RdRP amplicon comprises a sequence that is at least 85% identical to SEQ ID NO: 80 and/or SEQ ID NO: 81.
Embodiment 50. The method of any one of embodiments 47 to 49, wherein the method further comprises contacting the amplicons with at least one probe selected from a SARS-CoV-2 E probe, a SARS-CoV-2 N2 probe, and a SARS-CoV-2 RdRP probe, wherein
   a) the SARS-CoV-2 E probe comprises a sequence that is at least 85% identical or complementary to at least 15 contiguous nucleotides of SEQ ID NO: 72;
   b) the SARS-CoV-2 N2 probe comprises a sequence that is at least 85% identical or complementary to at least 15 contiguous nucleotides of SEQ ID NO: 74 and/or SEQ ID NO: 75; and
   c) the SARS-CoV-2 RdRP probe comprises a sequence that is at least 85% identical or complementary to at least 15 contiguous nucleotides of SEQ ID NO: 77.

In some embodiments, methods of detecting the presence or absence of SARS-CoV-2 and influenza A and/or influenza B in a sample from a subject are provided. The methods may further comprise detecting the presence or absence of an RSV gene.

In some embodiments, a method comprises detecting the presence or absence of a SARS-CoV-2 gene and at least one influenza A gene selected from polymerase acidic (PA), polymerase basic 2 (PB2), and MP in the sample. In some embodiments, a method comprises detecting the presence or absence of a SARS-CoV-2 gene selected from a SARS-CoV-2 envelope protein (E) gene, a SARS-CoV-2 nucleoprotein (N2 region in the N gene) gene, and a RNA-dependent RNA polymerase (RdRP) gene of the ORFlab sequence; and at least one influenza A gene selected from a polymerase acidic (PA) gene, a polymerase basic 2 (PB2), and MP in a sample from the subject. The methods may further comprise detecting the presence or absence of an RSV gene.

In some embodiments, a method comprises detecting the presence or absence of a SARS-CoV-2 gene and an influenza A PA gene. In some embodiments, a method comprises detecting the presence or absence of a SARS-CoV-2 gene and an influenza A PB2 gene. In some embodiments, a method comprises detecting the presence or absence of a SARS-CoV-2, influenza A PA gene, and a influenza A PB2 gene. In some embodiments, the sequence of the PA gene is at least 95% identical to the sequence of SEQ ID NO: 2. In some embodiments, the sequence of the PB2 gene is at least 95% identical to the sequence of SEQ ID NO: 1.

In some embodiments, the method further comprises detecting an influenza B gene, wherein the gene is selected from influenza B MP or influenza B NS.

In some embodiments, wherein the method comprises detecting the presence or absence of at least one influenza A and/or influenza B matrix protein (MP) gene, the sequence of the influenza A MP gene is at least 95% identical to the sequence of SEQ ID NO: 3. In some embodiments, the sequence of the influenza B MP gene is at least 95% identical to the sequence of SEQ ID NO: 6. In some embodiments, the method further comprises detecting the presence or absence of an avian influenza MP gene. In some embodiments, the sequence of the avian influenza MP gene is at least 95% identical to the sequence of SEQ ID NO: 4. In some embodiments, the avian influenza MP gene is a hemagglutinin (H) 5 or H7 subtype. In some embodiments, the method further comprises detecting the presence or absence of at least one influenza hemagglutinin (HA) gene. In some embodiments, the method comprises detecting the presence or absence of an influenza A HA gene. In some embodiments, the method comprises detecting the presence or absence of an avian influenza HA gene. In some embodiments, the avian influenza is an H7 subtype. In some embodiments, the sequence of the influenza HA gene is at least 95% identical to the sequence of SEQ ID NO: 5.

In some embodiments, the method further comprises detecting the presence or absence of at least one influenza nonstructural (NS) gene. In some embodiments, the method comprises detecting the presence or absence of an influenza B NS gene. In some embodiments, the sequence of the influenza B NS gene is at least 95% identical to the sequence of SEQ ID NO: 7.

In some embodiments, the method further comprises detecting the presence or absence of an influenza B MP gene and/or an influenza B NS gene. In some embodiments, the sequence of the influenza B MP gene is at least 95% identical to SEQ ID NO: 6 and the sequence of the influenza B NS gene is at least 95% identical to SEQ ID NO: 7.

In some embodiments, the method further comprises detecting the presence or absence of respiratory syncytial virus (RSV) in a sample from the subject. In some embodiments, the method comprises detecting the presence or absence of RSV A. In some embodiments, the method comprises detecting the presence or absence of RSV B. In some embodiments, the method comprises detecting the presence or absence of RSV A and RSV B.

In some embodiments, detection of the presence of any one of the SARS-CoV-2 genes indicates the presence of SARS-CoV-2 in the sample. In some embodiments, detection of the presence of any one of the influenza genes indicates the presence of influenza in the sample. In some embodiments, the method distinguishes between influenza A and influenza B. In some embodiments, the method does not distinguish between influenza A and influenza B. In some embodiments, detection of the presence of RSV A or RSV B indicates the presence of RSV in the sample.

In some embodiments, the method comprises detecting the presence or absence of a SARS-CoV-2 gene, an influenza A gene, and an influenza B gene. In some embodiments, the method comprises detecting the presence of or absence of a SARS-CoV-2 gene, an influenza A PA gene, an influenza A PB2 gene, an influenza A MP gene, and an avian influenza MP gene, and an avian influenza HA gene. In some embodiments, the sequence of the SARS-CoV-2 E gene is at least 95% identical to SEQ ID NO: 44, the sequence of the SARS-CoV-2 N2 gene is at least 95% identical to SEQ ID NO: 45, the sequence of the influenza A PA gene is at least 95% identical to SEQ ID NO: 2, the sequence of the influenza A PB2 gene is at least 95% identical to SEQ ID NO: 1, the sequence of the influenza A MP gene is at least 95% identical to SEQ ID NO: 3, the sequence of the avian influenza MP gene is at least 95% identical to SEQ ID NO: 4, and the sequence of the avian influenza HA gene is at least 95% identical to SEQ ID NO: 5.

In some embodiments, the subject has one or more symptoms of COVID-19, influenza, and/or RSV. In some embodiments, the subject has one or more symptoms selected from fever, chills, cough, shortness of breath or difficulty breathing, sore throat, runny nose, nasal congestion, muscle or body ache, headache, fatigue, new loss of taste or smell, nausea or vomiting, and diarrhea.

In some embodiments, the method comprises detecting an exogenous control. In some embodiments, the exogenous control is a sample processing control. In some embodiments, the exogenous control comprises an RNA sequence that is not expected to be present in the sample. In some embodiments, the exogenous control is an RNA control. In some embodiments, the RNA control is packaged in a bacteriophage protective coat (e.g., ARMORED® RNA).

In some embodiments, the method comprises PCR. In some embodiments, the method comprises quantitative PCR. In some embodiments, the PCR reaction takes less than 2 hours from an initial denaturation step through a final extension step. In some embodiments, the reaction take less than 2 hours, less than 1 hour, less than 45 minutes, less than 40 minutes, less than 35 minutes, or less than 30 minutes from initial denaturation through the last extension.

In some embodiments, the method comprises contacting nucleic acids from the sample with a primer pair for detecting the influenza A PA gene. In some embodiments, the primer pair comprises a first primer and a second primer, wherein the first primer comprises a sequence that is at least 85%, at least 90%, at least 95%, or 100% identical to at least 15, at least 16, at least 17, at least 18, at least 19, at least 20, at least 21, at least 22, at least 23, at least 24, or at least 25 contiguous nucleotides of SEQ ID NO: 2, and wherein the second primer comprises a sequence that is at least 85%, at least 90%, at least 95%, or 100% complementary to at least 15, at least 16, at least 17, at least 18, at least 19, at least 20, at least 21, at least 22, at least 23, at least 24, or at least 25 contiguous nucleotides of SEQ ID NO: 2. In some embodiments, the primer pair comprises a first primer and a second primer, wherein the first primer comprises a sequence that is at least 85%, at least 90%, at least 95%, or 100% identical to at least 15, at least 16, at least 17, at least 18, at least 19, at least 20, at least 21, at least 22, at least 23, at least 24, or at least 25 contiguous nucleotides of SEQ ID NO: 20, and wherein the second primer comprises a sequence that is at least 85%, at least 90%, at least 95%, or 100% identical to at least 15, at least 16, at least 17, at least 18, at least 19, or at least 20 contiguous nucleotides of SEQ ID NO: 21. In some embodiments, the first primer has the sequence of SEQ ID NO: 20 and the second primer has the sequence of SEQ ID NO: 21.

In some embodiments, the method comprises contacting nucleic acids from the sample with a primer pair for detecting the influenza A PB2 gene. In some embodiments, the primer pair comprises a primer comprising a sequence that is at least 85%, at least 90%, at least 95%, or 100% identical to at least 15, at least 16, at least 17, at least 18, at least 19, at least 20, at least 21, at least 22, at least 23, at least 24, or at least 25 contiguous nucleotides of SEQ ID NO: 1, and a primer comprising a sequence that is at least 85%, at least 90%, at least 95%, or 100% complementary to at least 15, at least 16, at least 17, at least 18, at least 19, at least 20, at least 21, at least 22, at least 23, at least 24, or at least 25 contiguous nucleotides of SEQ ID NO: 1. In some embodiments, the primer pair comprises a primer comprising a sequence that is at least 85%, at least 90%, at least 95%, or 100% identical to at least 15, at least 16, at least 17, at least 18, at least 19, or at least 20 contiguous nucleotides of SEQ ID NO: 17, and a primer comprising a sequence that is at least 85%, at least 90%, at least 95%, or 100% identical to at least 15, at least 16, at least 17, at least 18, at least 19, or at least 20 contiguous nucleotides of SEQ ID NO: 18. In some embodiments, the primer pair comprises a primer that comprises the sequence of SEQ ID NO: 17 and a primer comprising the sequence of SEQ ID NO: 18.

In some embodiments, the method comprises contacting nucleic acids from the sample with at least one additional primer pair, wherein each of the additional primer pairs is for detecting a different influenza gene selected from an influenza A MP gene, an avian influenza MP gene, and an avian influenza HA gene. In some embodiments, each additional primer pair comprises sets of primers independently selected from:
(a) a primer comprising a sequence that is at least 85%, at least 90%, at least 95%, or 100% identical to at least 15, at least 16, at least 17, at least 18, at least 19, at least 20, at least 21, at least 22, at least 23, at least 24, or at least 25 contiguous nucleotides of SEQ ID NO: 3, and a primer comprising a sequence that is at least 85%, at least 90%, at least 95%, or 100% complementary to at least 15, at least 16, at least 17, at least 18, at least 19, at least 20, at least 21, at least 22, at least 23, at least 24, or at least 25 contiguous nucleotides of SEQ ID NO: 3;
(b) a primer comprising a sequence that is at least 85%, at least 90%, at least 95%, or 100% identical to at least 15, at least 16, at least 17, at least 18, at least 19, at least 20, at least 21, at least 22, at least 23, at least 24, or at least 25 contiguous nucleotides of SEQ ID NO: 4, and a primer comprising a sequence that is at least 85%, at least 90%, at least 95%, or 100% complementary to at least 15, at least 16, at least 17, at least 18, at least 19, at least 20, at least 21, at least 22, at least 23, at least 24, or at least 25 contiguous nucleotides of SEQ ID NO: 4;
(c) a primer comprising a sequence that is at least 85%, at least 90%, at least 95%, or 100% identical to at least 15, at least 16, at least 17, at least 18, at least 19, at least 20, at least 21, at least 22, at least 23, at least 24, or at least 25 contiguous nucleotides of SEQ ID NO: 5, and a primer comprising a sequence that is at least 85%, at least 90%, at least 95%, or 100% complementary to at least 15, at least 16, at least 17, at least 18, at least 19, at least 20, at least 21, at least 22, at least 23, at least 24, or at least 25 contiguous nucleotides of SEQ ID NO: 5;
(d) a primer comprising a sequence that is at least 85%, at least 90%, at least 95%, or 100% identical to at least 15, at least 16, at least 17, at least 18, at least 19, at least 20, at least 21, at least 22, at least 23, at least 24, or at least 25 contiguous nucleotides of SEQ ID NO: 23, and a primer comprising a sequence that is at least 85%, at least 90%, at least 95%, or 100% identical to at least 15, at least 16, at least 17, at least 18, at least 19, at least 20, at least 21, at least 22, at least 23, at least 24, or at least 25 contiguous nucleotides of SEQ ID NO: 24; and (e) a primer comprising a sequence that is at least 85%, at least 90%, at least 95%, or 100% identical to at least 15, at least 16, at least 17, at least 18, at least 19, at least 20, at least 21, at least 22, at least 23, at least 24, or at least 25 contiguous nucleotides of SEQ ID NO: 26, and a primer comprising a sequence that is at least 85%, at least 90%, at least 95%, or 100% identical to at least 15, at least 16, at least 17, at least 18, at least 19, at least 20, at least 21, at least 22, at least 23, at least 24, or at least 25 contiguous nucleotides of SEQ ID NO: 27.

In some embodiments, the method comprises contacting nucleic acids from the sample with at least one additional primer pair, wherein each of the additional primer pairs is for detecting a different influenza gene selected from an influenza B MP gene and an influenza B NS gene. In some embodiments, each additional primer pair comprises a set of primers independently selected from:
(a) a primer comprising a sequence that is at least 85%, at least 90%, at least 95%, or 100% identical to at least 15, at least 16, at least 17, at least 18, at least 19, at least 20, at least 21, at least 22, at least 23, at least 24, or at least 25 contiguous nucleotides of SEQ ID NO: 6, and a primer comprising a sequence that is at least 85%, at least 90%, at least 95%, or 100% complementary to at least 15, at least 16, at least 17, at least 18, at least 19, at least 20, at least 21, at least 22, at least 23, at least 24, or at least 25 contiguous nucleotides of SEQ ID NO: 6;
(b) a primer comprising a sequence that is at least 85%, at least 90%, at least 95%, or 100% identical to at least 15, at least 16, at least 17, at least 18, at least 19, at least 20, at least 21, at least 22, at least 23, at least 24, or at least 25 contiguous nucleotides of SEQ ID NO: 7, and a primer comprising a sequence that is at least 85%, at least 90%, at least 95%, or 100% complementary to at least 15, at least 16, at least 17, at least 18, at least 19, at least 20, at least 21, at least 22, at least 23, at least 24, or at least 25 contiguous nucleotides of SEQ ID NO: 7;
(c) a primer comprising a sequence that is at least 85%, at least 90%, at least 95%, or 100% identical to at least 15, at least 16, at least 17, at least 18, at least 19, at least 20, at least 21, at least 22, at least 23, at least 24, or at least 25 contiguous nucleotides of SEQ ID NO: 32, and a primer comprising a sequence that is at least 85%, at least 90%, at least 95%, or 100% identical to at least 15, at least 16, at least 17, at least 18, at least 19, at least 20, at least 21, at least 22, at least 23, at least 24, or at least 25 contiguous nucleotides of SEQ ID NO: 33; and
(d) a primer comprising a sequence that is at least 85%, at least 90%, at least 95%, or 100% identical to at least 15, at least 16, at least 17, at least 18, at least 19, at least 20, at least 21, at least 22, at least 23, at least 24, or at least 25 contiguous nucleotides of SEQ ID NO: 35, and a primer comprising a sequence that is at least 85%, at least 90%, at least 95%, or 100% identical to at least 15, at least 16, at least 17, at least 18, at least 19, at least 20, at least 21, at least 22, at least 23, at least 24, or at least 25 contiguous nucleotides of SEQ ID NO: 36.

In some embodiments, the method comprises contacting nucleic acids from the sample with at least one additional primer pair, wherein each of the additional primer pairs is for detecting RSV A and/or RSV B. In some embodiments, each additional primer pair comprises a a set of primers independently selected from:
(a) at least one primer comprising a sequence that is at least 85%, at least 90%, at least 95%, or 100% identical to at least 15, at least 16, at least 17, at least 18, at least 19, at least 20, at least 21, at least 22, at least 23, at least 24, or at least 25 contiguous nucleotides of SEQ ID NO: 38 and/or SEQ ID NO: 67, and at least one primer comprising a sequence that is at least 85%, at least 90%, at least 95%, or 100% identical to at least 15, at least 16, at least 17, at least 18, at least 19, at least 20, at least 21, at least 22, at least 23, at least 24, or at least 25 contiguous nucleotides of SEQ ID NO: 39, SEQ ID NO: 68, and/or SEQ ID NO: 69; and
(b) a primer comprising a sequence that is at least 85%, at least 90%, at least 95%, or 100% identical to at least 15, at least 16, at least 17, at least 18, at least 19, at least 20, at least 21, at least 22, at least 23, at least 24, or at least 25 contiguous nucleotides of SEQ ID NO: 41, and a primer comprising a sequence that is at least 85%, at least 90%, at least 95%, or 100% identical to at least 15, at least 16, at least 17, at least 18, at least 19, at least 20, at least 21, at least 22, at least 23, at least 24, or at least 25 contiguous nucleotides of SEQ ID NO: 42.

In some embodiments, the method comprises contacting nucleic acids from the sample with at least one additional primer pair, wherein each of the additional primer pairs is for detecting SARS-CoV-2. In some embodiments, each additional primer pair comprises a a set of primers independently selected from:
(a) a primer comprising a sequence that is at least 85%, at least 90%, at least 95%, or 100% identical to at least 15, at least 16, at least 17, at least 18, at least 19, at least 20, at least 21, at least 22, at least 23, at least 24, or at least 25 contiguous nucleotides of SEQ ID NO: 44, and a primer comprising a sequence that is at least 85%, at least 90%, at least 95%, or 100% complementary to at least 15, at least 16, at least 17, at least 18, at least 19, at least 20, at least 21, at least 22, at least 23, at least 24, or at least 25 contiguous nucleotides of SEQ ID NO: 44; and
(b) a primer comprising a sequence that is at least 85%, at least 90%, at least 95%, or 100% identical to at least 15, at least 16, at least 17, at least 18, at least 19, at least 20, at least 21, at least 22, at least 23, at least 24, or at least 25 contiguous nucleotides of SEQ ID NO: 70 or SEQ ID NO: 48, and a primer comprising a sequence that is at least 85%, at least 90%, at least 95%, or 100% identical to at least 15, at least 16, at least 17, at least 18, at least 19, at least 20, at least 21, at least 22, at least 23, at least 24, or at least 25 contiguous nucleotides of SEQ ID NO: 71 or SEQ ID NO: 49;
(c) a primer comprising a sequence that is at least 85%, at least 90%, at least 95%, or 100% identical to at least 15, at least 16, at least 17, at least 18, at least 19, at least 20, at least 21, at least 22, at least 23, at least 24, or at least 25 contiguous nucleotides of SEQ ID NO: 45, and a primer comprising a sequence that is at least 85%, at least 90%, at least 95%, or 100% complementary to at least 15, at least 16, at least 17, at least 18, at least 19, at least 20, at least 21, at least 22, at least 23, at least 24, or at least 25 contiguous nucleotides of SEQ ID NO: 45; and
(d) a primer comprising a sequence that is at least 85%, at least 90%, at least 95%, or 100% identical to at least 15, at least 16, at least 17, at least 18, at least 19, at least 20, at least 21, at least 22, at least 23, at least 24, or at least 25 contiguous nucleotides of SEQ ID NO: 73 or SEQ ID NO: 51, and a primer comprising a sequence that is at least 85%, at least 90%, at least 95%, or 100% identical to at least 15, at least 16, at least 17, at least 18, at least 19, at least 20, at least 21, at least 22, at least 23, at least 24, or at least 25 contiguous nucleotides of SEQ ID NO: 52.
(e) a primer comprising a sequence that is at least 85%, at least 90%, at least 95%, or 100% identical to at least 15, at least 16, at least 17, at least 18, at least 19, at least 20, at least 21, at least 22, at least 23, at least 24, or at least 25 contiguous nucleotides of SEQ ID NO: 76, and at least one primer comprising a sequence that is at least 85%, at least 90%, at least 95%, or 100% identical to at least 15, at least 16, at least 17, at least 18, at least 19, at least 20, at least 21, at least 22, at least 23, at least 24, or at least 25 contiguous nucleotides of SEQ ID NO: 61 and/or SEQ ID NO: 78.

In some embodiments, the method comprises contacting nucleic acids from the sample with primer pairs for detecting an influenza A gene, an influenza B gene, a SARS-CoV-2 gene and an RSV gene. In some embodiments, at least one forward primer and/or at least one reverse primer is used to detect the gene. In some embodiments, the method comprises contacting nucleic acids from the sample with primer pairs for detecting SARS-CoV-2 and one or more genes selected from an influenza A gene, an influenza B gene, and an RSV gene. In some embodiments, the method comprises contacting nucleic acids from the sample with primer pairs for detecting SARS-CoV-2 E and/or SARS-CoV-2 N2 and/or SARS-CoV-2 RdRP. In some embodiments, the method comprises contacting nucleic acids from the sample with primer pairs for detecting an influenza A PA gene, an influenza A PB2 gene, an influenza A MP gene, an avian influenza MP gene, and an avian influenza HA gene. In some embodiments, the method further comprises contacting nucleic acids from the sample with primer pairs for detecting an influenza B MP gene and an influenza B NS gene. In some embodiments, the method further comprises contacting nucleic acids from the sample with primer pairs for detecting RSV A and RSV B.

In some embodiments, the method comprises contacting nucleic acids from the sample with a control primer pair for detecting an exogenous control.

In some embodiments, each primer pair produces an amplicon that is 50 to 500 nucleotides long, 50 to 400 nucleotides long, 50 to 300 nucleotides long, 50 to 200 nucleotides long, or 50 to 150 nucleotides long.

In some embodiments, the method comprises forming an amplicon from each primer pair when the target of the primer pair is present. In some embodiments, the method comprises forming at least one amplicon selected from an influenza A PA amplicon, an influenza A PB2 amplicon. In some embodiments, the influenza A PA amplicon has the sequence of SEQ ID NO: 9 and the influenza A PB2 amplicon comprises the sequence of SEQ ID NO: 8.

In some embodiments, the method comprises forming at least one amplicon selected from an influenza A MP amplicon, an avian influenza MP amplicon, and an avian influenza HA amplicon. In some embodiments, the influenza A MP amplicon comprises the sequence of SEQ ID NO: 10, the avian influenza MP amplicon comprises the sequence of SEQ ID NO: 11, and the avian influenza HA amplicon comprises the sequence of SEQ ID NO: 12.

In some embodiments, the method further comprises forming an influenza B MP amplicon and/or an influenza B NS amplicon. In some embodiments, the influenza B MP amplicon comprises the sequence of SEQ ID NO: 13 and the influenza B NS amplicon comprises the sequence of SEQ ID NO: 14. In some embodiments, the method further comprises forming an RSV A amplicon and/or an RSV B amplicon. In some embodiments, the RSV A amplicon has the sequence of SEQ ID NO: 15 and the RSV B amplicon comprises the sequence of SEQ ID NO: 16. In some embodiments, the method further comprises forming at least one SARS-CoV-2 E and/or SARS-CoV-2 N2 and/or SARS-CoV-2 RdRP amplicon. In some embodiments, the SARS-CoV-2 E amplicon comprises the sequence of SEQ ID NO: 46 or SEQ ID NO: 79, the SARS-CoV-2 N2 amplicon comprises the sequence of SEQ ID NO: 47, and the SARS-CoV-2 RdRP amplicon comprises the sequence of SEQ ID NO: 80 and/or SEQ ID NO: 81.

In some embodiments, the method comprises contacting the amplicons with at least one probe selected from an influenza A PA probe and an influenza A PB2 probe. In some embodiments, the influenza PA probe comprises a sequence that is at least 85%, at least 90%, at least 95%, or 100% identical or complementary to at least 15, at least 16, at least 17, at least 18, at least 19, at least 20, at least 21, at least 22, at least 23, at least 24, or at least 25 contiguous nucleotides of SEQ ID NO: 2, and the influenza PB2 probe comprises a sequence that is at least 85%, at least 90%, at least 95%, or 100% identical or complementary to at least 15, at least 16, at least 17, at least 18, at least 19, at least 20, at least 21, at least 22, at least 23, at least 24, or at least 25 contiguous nucleotides of SEQ ID NO: 1. In some embodiments, the influenza PA probe comprises a sequence that is at least 85%, at least 90%, at least 95%, or 100% identical or complementary to at least 15, at least 16, at least 17, at least 18, at least 19, at least 20, at least 21, at least 22, at least 23, at least 24, or at least 25 contiguous nucleotides of SEQ ID NO: 22, and the influenza PB2 probe comprises a sequence that is at least 85%, at least 90%, at least 95%, or 100% identical or complementary to at least 15, at least 16, at least 17, at least 18, at least 19, at least 20, at least 21, at least 22, at least 23, at least 24, or at least 25 contiguous nucleotides of SEQ ID NO: 19.

In some embodiments, the method comprises contacting the amplicons with at least one probe selected from an influenza A MP probe, an avian influenza MP probe, and an avian influenza HA probe. In some embodiments, the influenza MP probe comprises a sequence that is at least 85%, at least 90%, at least 95%, or 100% identical or complementary to at least 15, at least 16, at least 17, at least 18, at least 19, at least 20, at least 21, at least 22, at least 23, at least 24, or at least 25 contiguous nucleotides of SEQ ID NO: 3, and the avian influenza MP probe comprises a sequence that is at least 85%, at least 90%, at least 95%, or 100% identical or complementary to at least 15, at least 16, at least 17, at least 18, at least 19, at least 20, at least 21, at least 22, at least 23, at least 24, or at least 25 contiguous nucleotides of SEQ ID NO: 4, and the avian influenza HA probe comprises a sequence that is at least 85%, at least 90%, at least 95%, or 100% identical or complementary to at least 15, at least 16, at least 17, at least 18, at least 19, at least 20, at least 21, at least 22, at least 23, at least 24, or at least 25 contiguous nucleotides of SEQ ID NO: 5. In some embodiments, the influenza MP probe comprises a sequence that is at least 85%, at least 90%, at least 95%, or 100% identical or complementary to at least 15, at least 16, at least 17, at least 18, at least 19, at least 20, at least 21, at least 22, at least 23, at least 24, or at least 25 contiguous nucleotides of SEQ ID NO: 25, and the avian influenza MP probe comprises a sequence that is at least 85%, at least 90%, at least 95%, or 100% identical or complementary to at least 15, at least 16, at least 17, at least 18, at least 19, at least 20, at least 21, at least 22, at least 23, at least 24, or at least 25 contiguous nucleotides of SEQ ID NO: 28.

In some embodiments, the method comprises contacting the amplicons with at least one probe selected from an influenza B MP probe and an influenza B NS probe. In some embodiments, the influenza B MP probe comprises a sequence that is at least 85%, at least 90%, at least 95%, or 100% identical or complementary to at least 15, at least 16, at least 17, at least 18, at least 19, at least 20, at least 21, at least 22, at least 23, at least 24, or at least 25 contiguous nucleotides of SEQ ID NO: 6, and the influenza B NS probe comprises a sequence that is at least 85%, at least 90%, at least 95%, or 100% identical or complementary to at least 15, at least 16, at least 17, at least 18, at least 19, at least 20, at least 21, at least 22, at least 23, at least 24, or at least 25 contiguous nucleotides of SEQ ID NO: 7. In some embodiments, the influenza B MP probe comprises a sequence that is at least 85%, at least 90%, at least 95%, or 100% identical or complementary to at least 15, at least 16, at least 17, at least 18, at least 19, at least 20, at least 21, at least 22, at least 23, at least 24, or at least 25 contiguous nucleotides of SEQ ID NO: 34, and the influenza B NS probe comprises a sequence that is at least 85%, at least 90%, at least 95%, or 100% identical or complementary to at least 15, at least 16, at least 17, at least 18, at least 19, at least 20, at least 21, at least 22, at least 23, at least 24, or at least 25 contiguous nucleotides of SEQ ID NO: 37.

In some embodiments, the method comprises contacting the amplicons with at least one probe selected from an RSV A probe and an RSV B probe. In some embodiments, the RSV A probe comprises a sequence that is at least 85%, at least 90%, at least 95%, or 100% identical or complementary to at least 15, at least 16, at least 17, at least 18, at least 19, at least 20, at least 21, at least 22, at least 23, at least 24, or at least 25 contiguous nucleotides of SEQ ID NO: 15, and the RSV B probe comprises a sequence that is at least 85%, at least 90%, at least 95%, or 100% identical or complementary to at least 15, at least 16, at least 17, at least 18, at least 19, at least 20, at least 21, at least 22, at least 23, at least 24, or at least 25 contiguous nucleotides of SEQ ID NO: 16. In some embodiments, the RSV A probe comprises a sequence that is at least 85%, at least 90%, at least 95%, or 100% identical or complementary to at least 15, at least 16, at least 17, at least 18, at least 19, at least 20, at least 21, at least 22, at least 23, at least 24, or at least 25 contiguous nucleotides of SEQ ID NO: 40, and the RSV B probe comprises a sequence that is at least 85%, at least 90%, at least 95%, or 100% identical or complementary to at least 15, at least 16, at least 17, at least 18, at least 19, at least 20, at least 21, at least 22, at least 23, at least 24, or at least 25 contiguous nucleotides of SEQ ID NO: 43.

In some embodiments, the method comprises contacting the amplicons with at least one probe selected from a SARS-CoV-2 E probe, a SARS-CoV-2 N2 probe, and a SARS-CoV-2 RdRP probe. In some embodiments, the SARS-CoV-2 E probe comprises a sequence that is at least 85%, at least 90%, at least 95%, or 100% identical or complementary to at least 15, at least 16, at least 17, at least 18, at least 19, at least 20, at least 21, at least 22, at least 23, at least 24, or at least 25 contiguous nucleotides of SEQ ID NO: 44, and the SARS-CoV-2 N2 probe comprises a sequence that is at least 85%, at least 90%, at least 95%, or 100% identical or complementary to at least 15, at least 16, at least 17, at least 18, at least 19, at least 20, at least 21, at least 22, at least 23, at least 24, or at least 25 contiguous nucleotides of SEQ ID NO: 45 or SEQ ID NO: 79. In some embodiments, the SARS-CoV-2 RdRP probe comprises a sequence that is at least 85%, at least 90%, at least 95%, or 100% identical or complementary to at least 15, at least 16, at least 17, at least 18, at least 19, at least 20, at least 21, at least 22, at least 23, at least 24, or at least 25 contiguous nucleotides of SEQ ID NO: 80 and/or SEQ ID NO: 81. In some embodiments, the SARS-CoV-2 E probe comprises a sequence that is at least 85%, at least 90%, at least 95%, or 100% identical or complementary to at least 15, at least 16, at least 17, at least 18, at least 19, at least 20, or at least 21, contiguous nucleotides of SEQ ID NO: 50 or SEQ ID NO: 72, and the SARS-CoV-2 N2 probe comprises a sequence that is at least 85%, at least 90%, at least 95%, or 100% identical or complementary to at least 15, at least 16, at least 17, at least 18, at least 19, at least 20, at least 21, at least 22, at least 23, or at least 24 contiguous nucleotides of SEQ ID NO: 53, SEQ ID NO: 74 and/or SEQ ID NO: 75. In some embodiments, the SARS-CoV-2 RdRP probe comprises a sequence that is at least 85%, at least 90%, at least 95%, or 100% identical or complementary to at least 15, at least 16, at least 17, at least 18, at least 19, at least 20, or at least 21, contiguous nucleotides of SEQ ID NO: 77. In some embodiments two probes are included for SARS-CoV-2 N2, a first probe that comprises a sequence that is at least 85%, at least 90%, at least 95%, or 100% identical or complementary to at least 15, at least 16, at least 17, at least 18, at least 19, at least 20, at least 21, at least 22, at least 23, or at least 24 contiguous nucleotides of SEQ ID NO: 53 or SEQ ID NO: 74, and a second probe that comprises a sequence that is at least 85%, at least 90%, at least 95%, or 100% identical or complementary to at least 15, at least 16, at least 17, at least 18, at least 19, at least 20, at least 21, at least 22, at least 23, or at least 24 contiguous nucleotides of SEQ ID NO: 75.

In some embodiments, each probe comprises a detectable label. In some embodiments, the each probe comprises a fluorescent dye and a quencher molecule. In some embodiments, the probes comprise detectable labels that are detectably different. In some embodiments, the probes comprise detectable labels that are not detectably different. In some embodiments, each probe consists of 15 to 30 nucleotides. In some embodiments the probes for a single organism are not detectably different and probes for different organisms are detectably different.

In some embodiments, the method comprises forming an exogenous control amplicon. In some embodiments, the method comprises contacting the exogenous control amplicon with a control probe capable of selectively hybridizing with the exogenous control amplicon.

In some embodiments, the method comprises detecting the presence or absence of at least one influenza A subtype and at least one influenza B subtype and an exogenous control in a single multiplex reaction. In some embodiments, the at least one influenza A subtype includes at least one avian influenza. In some embodiments, the method further comprises detecting RSV A and/or RSV B and SARS-CoV-2 E and/or SARS-CoV-2 N2 and/or SARS-CoV-2 RdRP in the same multiplex reaction. In some embodiments, using the multiplex reaction results in conservation of test materials that may have limited availability or results in avoiding increased cost to run the assays as single tests, thus increasing the availability of testing and reducing the cost of testing.

In some embodiments, the sample is selected from a nasopharyngeal swab sample, a nasal aspirate sample, and a nasal wash sample.

In some embodiments, compositions are provided. In some embodiments, a composition comprises a first primer pair for detecting an influenza PA gene. In some embodiments, the first primer pair comprises a first primer and a second primer, wherein the first primer comprises a sequence that is at least 85%, at least 90%, at least 95%, or 100% identical to at least 15, at least 16, at least 17, at least 18, at least 19, at least 20, at least 21, at least 22, at least 23, at least 24, or at least 25 contiguous nucleotides of SEQ ID NO: 2, and wherein the second primer comprises a sequence that is at least 85%, at least 90%, at least 95%, or 100% complementary to at least 15, at least 16, at least 17, at least 18, at least 19, at least 20, at least 21, at least 22, at least 23, at least 24, or at least 25 contiguous nucleotides of SEQ ID NO: 2. In some embodiments, the first primer pair comprises a first primer and a second primer, wherein the first primer comprises a sequence that is at least 85%, at least 90%, at least 95%, or 100% identical to at least 15, at least 16, at least 17, at least 18, at least 19, at least 20, at least 21, at least 22, at least 23, at least 24, or at least 25 contiguous nucleotides of SEQ ID NO: 20, and wherein the second primer comprises a sequence that is at least 85%, at least 90%, at least 95%, or 100% identical to at least 15, at least 16, at least 17, at least 18, at least 19, or at least 20 contiguous nucleotides of SEQ ID NO: 21. In some embodiments, the first primer has the sequence of SEQ ID NO: 20 and the second primer has the sequence of SEQ ID NO: 21.

In some embodiments, a composition comprising a second primer pair for detecting an influenza PB2 gene is provided. In some embodiments, the second primer pair comprises a third primer and a fourth primer, wherein the third primer comprises a sequence that is at least 85%, at least 90%, at least 95%, or 100% identical to at least 15, at least 16, at least 17, at least 18, at least 19, at least 20, at least 21, at least 22, at least 23, at least 24, or at least 25 contiguous nucleotides of SEQ ID NO: 1, and wherein the fourth primer comprises a sequence that is at least 85%, at least 90%, at least 95%, or 100% complementary to at least 15, at least 16, at least 17, at least 18, at least 19, at least 20, at least 21, at least 22, at least 23, at least 24, or at least 25 contiguous nucleotides of SEQ ID NO: 1. In some embodiments, the second primer pair comprises a third primer and a fourth primer, wherein the third primer comprises a sequence that is at least 85%, at least 90%, at least 95%, or 100% identical to at least 15, at least 16, at least 17, at least 18, at least 19, or at least 20 contiguous nucleotides of SEQ ID NO: 17, and wherein the fourth primer comprises a sequence that is at least 85%, at least 90%, at least 95%, or 100% identical to at least 15, at least 16, at least 17, at least 18, at least 19, or at least 20 contiguous nucleotides of SEQ ID NO: 18. In some embodiments, the third primer has the sequence of SEQ ID NO: 17 and the fourth primer has the sequence of SEQ ID NO: 18.

In some embodiments, a composition comprises at least one additional primer pair, wherein each of the additional primer pairs is for detecting a different influenza gene selected from an influenza A MP gene, an avian influenza MP gene, and an avian influenza HA gene. In some embodiments, each additional primer pair comprises a fifth primer and a sixth primer independently selected from: (a) a fifth primer comprising a sequence that is at least 85%, at least 90%, at least 95%, or 100% identical to at least 15, at least 16, at least 17, at least 18, at least 19, at least 20, at least 21, at least 22, at least 23, at least 24, or at least 25 contiguous nucleotides of SEQ ID NO: 3, and a sixth primer comprising a sequence that is at least 85%, at least 90%, at least 95%, or 100% complementary to at least 15, at least 16, at least 17, at least 18, at least 19, at least 20, at least 21, at least 22, at least 23, at least 24, or at least 25 contiguous nucleotides of SEQ ID NO: 3; (b) a fifth primer comprising a sequence that is at least 85%, at least 90%, at least 95%, or 100% identical to at least 15, at least 16, at least 17, at least 18, at least 19, at least 20, at least 21, at least 22, at least 23, at least 24, or at least 25 contiguous nucleotides of SEQ ID NO: 4, and a sixth primer comprising a sequence that is at least 85%, at least 90%, at least 95%, or 100% complementary to at least 15, at least 16, at least 17, at least 18, at least 19, at least 20, at least 21, at least 22, at least 23, at least 24, or at least 25 contiguous nucleotides of SEQ ID NO: 4; (c) a fifth primer comprising a sequence that is at least 85%, at least 90%, at least 95%, or 100% identical to at least 15, at least 16, at least 17, at least 18, at least 19, at least 20, at least 21, at least 22, at least 23, at least 24, or at least 25 contiguous nucleotides of SEQ ID NO: 5, and a sixth primer comprising a sequence that is at least 85%, at least 90%, at least 95%, or 100% complementary to at least 15, at least 16, at least 17, at least 18, at least 19, at least 20, at least 21, at least 22, at least 23, at least 24, or at least 25 contiguous nucleotides of SEQ ID NO: 5; (d) a fifth primer comprising a sequence that is at least 85%, at least 90%, at least 95%, or 100% identical to at least 15, at least 16, at least 17, at least 18, at least 19, at least 20, at least 21, at least 22, at least 23, at least 24, or at least 25 contiguous nucleotides of SEQ ID NO: 23, and a sixth primer comprising a sequence that is at least 85%, at least 90%, at least 95%, or 100% identical to at least 15, at least 16, at least 17, at least 18, at least 19, at least 20, at least 21, at least 22, at least 23, at least 24, or at least 25 contiguous nucleotides of SEQ ID NO: 24; and (e) a fifth primer comprising a sequence that is at least 85%, at least 90%, at least 95%, or 100% identical to at least 15, at least 16, at least 17, at least 18, at least 19, at least 20, at least 21, at least 22, at least 23, at least 24, or at least 25 contiguous nucleotides of SEQ ID NO: 26, and a sixth primer comprising a sequence that is at least 85%, at least 90%, at least 95%, or 100% identical to at least 15, at least 16, at least 17, at least 18, at least 19, at least 20, at least 21, at least 22, at least 23, at least 24, or at least 25 contiguous nucleotides of SEQ ID NO: 27.

In some embodiments, the composition further comprises at least one additional primer pair, wherein each of the additional primer pairs is for detecting a different influenza gene selected from an influenza B MP gene and an influenza B NS gene. In some embodiments, each additional primer pair comprises a seventh primer and an eighth primer independently selected from: (a) a seventh primer comprising a sequence that is at least 85%, at least 90%, at least 95%, or 100% identical to at least 15, at least 16, at least 17, at least 18, at least 19, at least 20, at least 21, at least 22, at least 23, at least 24, or at least 25 contiguous nucleotides of SEQ ID NO: 6, and an eighth primer comprising a sequence that is at least 85%, at least 90%, at least 95%, or 100% complementary to at least 15, at least 16, at least 17, at least 18, at least 19, at least 20, at least 21, at least 22, at least 23, at least 24, or at least 25 contiguous nucleotides of SEQ ID NO: 6; (b) a seventh primer comprising a sequence that is at least 85%, at least 90%, at least 95%, or 100% identical to at least 15, at least 16, at least 17, at least 18, at least 19, at least 20, at least 21, at least 22, at least 23, at least 24, or at least 25 contiguous nucleotides of SEQ ID NO: 7, and an eighth primer comprising a sequence that is at least 85%, at least 90%, at least 95%, or 100% complementary to at least 15, at least 16, at least 17, at least 18, at least 19, at least 20, at least 21, at least 22, at least 23, at least 24, or at least 25 contiguous nucleotides of SEQ ID NO: 7; (c) a seventh primer comprising a sequence that is at least 85%, at least 90%, at least 95%, or 100% identical to at least 15, at least 16, at least 17, at least 18, at least 19, at least 20, at least 21, at least 22, at least 23, at least 24, or at least 25 contiguous nucleotides of SEQ ID NO: 32, and an eighth primer comprising a sequence that is at least 85%, at least 90%, at least 95%, or 100% identical to at least 15, at least 16, at least 17, at least 18, at least 19, at least 20, at least 21, at least 22, at least 23, at least 24, or at least 25 contiguous nucleotides of SEQ ID NO: 33; and (d) a seventh primer comprising a sequence that is at least 85%, at least 90%, at least 95%, or 100% identical to at least 15, at least 16, at least 17, at least 18, at least 19, at least 20, at least 21, at least 22, at least 23, at least 24, or at least 25 contiguous nucleotides of SEQ ID NO: 35, and an eighth primer comprising a sequence that is at least 85%, at least 90%, at least 95%, or 100% identical to at least 15, at least 16, at least 17, at least 18, at least 19, at least 20, at least 21, at least 22, at least 23, at least 24, or at least 25 contiguous nucleotides of SEQ ID NO: 36.

In some embodiments, a composition further comprises at least one additional primer pair, wherein each of the additional primer pairs is for detecting RSV A or RSV B. In some embodiments, each additional primer pair comprises a ninth primer and a tenth primer independently selected from: (a) a ninth primer comprising a sequence that is at least 85%, at least 90%, at least 95%, or 100% identical to at least 15, at least 16, at least 17, at least 18, at least 19, at least 20, at least 21, at least 22, at least 23, at least 24, or at least 25 contiguous nucleotides of SEQ ID NO: 38 and/or SEQ ID NO: 67, and a tenth primer comprising a sequence that is at least 85%, at least 90%, at least 95%, or 100% identical to at least 15, at least 16, at least 17, at least 18, at least 19, at least 20, at least 21, at least 22, at least 23, at least 24, or at least 25 contiguous nucleotides of SEQ ID NO: 39, SEQ ID NO: 68, and/or SEQ ID NO: 69; and (b) a ninth primer comprising a sequence that is at least 85%, at least 90%, at least 95%, or 100% identical to at least 15, at least 16, at least 17, at least 18, at least 19, at least 20, at least 21, at least 22, at least 23, at least 24, or at least 25 contiguous nucleotides of SEQ ID NO: 41, and a tenth primer comprising a sequence that is at least 85%, at least 90%, at least 95%, or 100% identical to at least 15, at least 16, at least 17, at least 18, at least 19, at least 20, at least 21, at least 22, at least 23, at least 24, or at least 25 contiguous nucleotides of SEQ ID NO: 42. In some embodiments at least two forward primers and/or at least two reverse primers may be used to detect RSV A and/or RSV B. In some embodiments, the primers used to detect RSV A comprise a forward primer comprising a sequence that is at least 85% identical to at least 15 contiguous nucleotides of SEQ ID NO: 38 and/or a forward primer comprising a sequence that is at least 85% identical to at least 15 contiguous nucleotides of SEQ ID NO: 67; and a reverse primer comprising a sequence that is at least 85% complementary to at least 15 contiguous nucleotides of SEQ ID NO: 39 and/or a reverse primer comprising a sequence that is at least 85% complementary to at least 15 contiguous nucleotides of SEQ ID NO: 68 and/or a reverse primer comprising a sequence that is at least 85% complementary to at least 15 contiguous nucleotides of SEQ ID NO: 69.

In some embodiments, a composition further comprises at least one additional primer pair, wherein each of the additional primer pairs is for detecting the SARS-CoV-2 E gene and/or SARS-CoV-2 N2 gene and/or SARS-CoV-2 RdRP gene. In some embodiments, each additional primer pair comprises an eleventh primer and a twelfth primer independently selected from: (a) an eleventh primer comprising a sequence that is at least 85%, at least 90%, at least 95%, or 100% identical to at least 15, at least 16, at least 17, at least 18, at least 19, at least 20, at least 21, at least 22, at least 23, at least 24, or at least 25 contiguous nucleotides of SEQ ID NO: 44, and a twelfth primer comprising a sequence that is at least 85%, at least 90%, at least 95%, or 100% complementary to at least 15, at least 16, at least 17, at least 18, at least 19, at least 20, at least 21, at least 22, at least 23, at least 24, or at least 25 contiguous nucleotides of SEQ ID NO: 44; (b) an eleventh primer comprising a sequence that is at least 85%, at least 90%, at least 95%, or 100% identical to at least 15, at least 16, at least 17, at least 18, at least 19, at least 20, at least 21, at least 22, at least 23, at least 24, or at least 25 contiguous nucleotides of SEQ ID NO: 70, and a twelfth primer comprising a sequence that is at least 85%, at least 90%, at least 95%, or 100% complementary to at least 15, at least 16, at least 17, at least 18, at least 19, at least 20, at least 21, at least 22, at least 23, at least 24, or at least 25 contiguous nucleotides of SEQ ID NO: 71; (c) an eleventh primer comprising a sequence that is at least 85%, at least 90%, at least 95%, or 100% identical to at least 15, at least 16, at least 17, at least 18, at least 19, at least 20, at least 21, at least 22, at least 23, at least 24, or at least 25 contiguous nucleotides of SEQ ID NO: 48, and a twelfth primer comprising a sequence that is at least 85%, at least 90%, at least 95%, or 100% identical to at least 15, at least 16, at least 17, at least 18, at least 19, at least 20, at least 21, at least 22, at least 23, at least 24, or at least 25 contiguous nucleotides of SEQ ID NO: 49; (d) an eleventh primer comprising a sequence that is at least 85%, at least 90%, at least 95%, or 100% identical to at least 15, at least 16, at least 17, at least 18, at least 19, at least 20, at least 21, at least 22, at least 23, at least 24, or at least 25 contiguous nucleotides of SEQ ID NO: 45, and a twelfth primer comprising a sequence that is at least 85%, at least 90%, at least 95%, or 100% complementary to at least 15, at least 16, at least 17, at least 18, at least 19, at least 20, at least 21, at least 22, at least 23, at least 24, or at least 25 contiguous nucleotides of SEQ ID NO: 45; (e) an eleventh primer comprising a sequence that is at least 85%, at least 90%, at least 95%, or 100% identical to at least 15, at least 16, at least 17, at least 18, at least 19, at least 20, at least 21, at least 22, at least 23, at least 24, or at least 25 contiguous nucleotides of SEQ ID No: 73 or SEQ ID NO: 51, and a twelfth primer comprising a sequence that is at least 85%, at least 90%, at least 95%, or 100% identical to at least 15, at least 16, at least 17, at least 18, at least 19, at least 20, at least 21, at least 22, at least 23, at least 24, or at least 25 contiguous nucleotides of SEQ ID NO: 52; and (f) an eleventh primer comprising a sequence that is at least 85%, at least 90%, at least 95%, or 100% identical to at least 15, at least 16, at least 17, at least 18, at least 19, at least 20, at least 21, at least 22, at least 23, at least 24, or at least 25 contiguous nucleotides of SEQ ID NO: 76, and a twelfth primer comprising a sequence that is at least 85%, at least 90%, at least 95%, or 100% identical to at least 15, at least 16, at least 17, at least 18, at least 19, at least 20, at least 21, at least 22, at least 23, at least 24, or at least 25 contiguous nucleotides of SEQ ID NO: 61 and/or SEQ ID NO: 78.

In some embodiments, a composition comprises primer pairs for detecting at least one influenza A gene, influenza B gene, SARS-CoV-2 gene and RSV gene. In some embodiments, the composition comprises primer pairs for detecting SARS-CoV-2 E and/or SARS-CoV-2 N2 and/or SARS-CoV-2 RdRP. In some embodiments, the composition comprises primer pairs for detecting an influenza A PA gene, an influenza A PB2 gene, an influenza A MP gene, and/or an avian influenza MP gene. In some embodiments, the composition further comprises primer pairs for detecting an influenza B MP gene and/or an influenza B NS gene. In some embodiments, the composition further comprises primer pairs for detecting RSV A and/or RSV B. In some embodiments, the composition further comprises a primer pair for detecting an exogenous control. In some embodiments, the exogenous control is a sample processing control.

In some embodiments, a composition comprises at least one probe selected from an influenza A PA probe and an influenza A PB2 probe. In some embodiments, the influenza PA probe comprises a sequence that is at least 85%, at least 90%, at least 95%, or 100% identical or complementary to at least 15, at least 16, at least 17, at least 18, at least 19, at least 20, at least 21, at least 22, at least 23, at least 24, or at least 25 contiguous nucleotides of SEQ ID NO: 2, and the influenza PB2 probe comprises a sequence that is at least 85%, at least 90%, at least 95%, or 100% identical or complementary to at least 15, at least 16, at least 17, at least 18, at least 19, at least 20, at least 21, at least 22, at least 23, at least 24, or at least 25 contiguous nucleotides of SEQ ID NO: 1. In some embodiments, the influenza PA probe comprises a sequence that is at least 85%, at least 90%, at least 95%, or 100% identical or complementary to at least 15, at least 16, at least 17, at least 18, at least 19, at least 20, at least 21, at least 22, at least 23, at least 24, or at least 25 contiguous nucleotides of SEQ ID NO: 22, and the influenza PB2 probe comprises a sequence that is at least 85%, at least 90%, at least 95%, or 100% identical or complementary to at least 15, at least 16, at least 17, at least 18, at least 19, at least 20, at least 21, at least 22, at least 23, at least 24, or at least 25 contiguous nucleotides of SEQ ID NO: 19.

In some embodiments, a composition further comprises at least one probe selected from an influenza A MP probe, an avian influenza MP probe, and an avian influenza HA probe. In some embodiments, the influenza MP probe comprises a sequence that is at least 85%, at least 90%, at least 95%, or 100% identical or complementary to at least 15, at least 16, at least 17, at least 18, at least 19, at least 20, at least 21, at least 22, at least 23, at least 24, or at least 25 contiguous nucleotides of SEQ ID NO: 3, and the avian influenza MP probe comprises a sequence that is at least 85%, at least 90%, at least 95%, or 100% identical or complementary to at least 15, at least 16, at least 17, at least 18, at least 19, at least 20, at least 21, at least 22, at least 23, at least 24, or at least 25 contiguous nucleotides of SEQ ID NO: 4, and the avian influenza HA probe comprises a sequence that is at least 85%, at least 90%, at least 95%, or 100% identical or complementary to at least 15, at least 16, at least 17, at least 18, at least 19, at least 20, at least 21, at least 22, at least 23, at least 24, or at least 25 contiguous nucleotides of SEQ ID NO: 5. In some embodiments, the influenza MP probe comprises a sequence that is at least 85%, at least 90%, at least 95%, or 100% identical or complementary to at least 15, at least 16, at least 17, at least 18, at least 19, at least 20, at least 21, at least 22, at least 23, at least 24, or at least 25 contiguous nucleotides of SEQ ID NO: 25, and the avian influenza MP probe comprises a sequence that is at least 85%, at least 90%, at least 95%, or 100% identical or complementary to at least 15, at least 16, at least 17, at least 18, at least 19, at least 20, at least 21, at least 22, at least 23, at least 24, or at least 25 contiguous nucleotides of SEQ ID NO: 28.

In some embodiments, the composition further comprises at least one probe selected from an influenza B MP probe and an influenza B NS probe. In some embodiments, the influenza B MP probe comprises a sequence that is at least 85%, at least 90%, at least 95%, or 100% identical or complementary to at least 15, at least 16, at least 17, at least 18, at least 19, at least 20, at least 21, at least 22, at least 23, at least 24, or at least 25 contiguous nucleotides of SEQ ID NO: 6, and the influenza B NS probe comprises a sequence that is at least 85%, at least 90%, at least 95%, or 100% identical or complementary to at least 15, at least 16, at least 17, at least 18, at least 19, at least 20, at least 21, at least 22, at least 23, at least 24, or at least 25 contiguous nucleotides of SEQ ID NO: 7. In some embodiments, the influenza B MP probe comprises a sequence that is at least 85%, at least 90%, at least 95%, or 100% identical or complementary to at least 15, at least 16, at least 17, at least 18, at least 19, at least 20, at least 21, at least 22, at least 23, at least 24, or at least 25 contiguous nucleotides of SEQ ID NO: 34, and the influenza B NS probe comprises a sequence that is at least 85%, at least 90%, at least 95%, or 100% identical or complementary to at least 15, at least 16, at least 17, at least 18, at least 19, at least 20, at least 21, at least 22, at least 23, at least 24, or at least 25 contiguous nucleotides of SEQ ID NO: 37.

In some embodiments, the composition further comprises at least one probe selected from an RSV A probe and an RSV B probe. In some embodiments, the RSV A probe comprises a sequence that is at least 85%, at least 90%, at least 95%, or 100% identical or complementary to at least 15, at least 16, at least 17, at least 18, at least 19, at least 20, at least 21, at least 22, at least 23, at least 24, or at least 25 contiguous nucleotides of SEQ ID NO: 15, and the influenza B NS probe comprises a sequence that is at least 85%, at least 90%, at least 95%, or 100% identical or complementary to at least 15, at least 16, at least 17, at least 18, at least 19, at least 20, at least 21, at least 22, at least 23, at least 24, or at least 25 contiguous nucleotides of SEQ ID NO: 16. In some embodiments, the RSV A probe comprises a sequence that is at least 85%, at least 90%, at least 95%, or 100% identical or complementary to at least 15, at least 16, at least 17, at least 18, at least 19, at least 20, at least 21, at least 22, at least 23, at least 24, or at least 25 contiguous nucleotides of SEQ ID NO: 40, and the RSV B probe comprises a sequence that is at least 85%, at least 90%, at least 95%, or 100% identical or complementary to at least 15, at least 16, at least 17, at least 18, at least 19, at least 20, at least 21, at least 22, at least 23, at least 24, or at least 25 contiguous nucleotides of SEQ ID NO: 43.

In some embodiments, the composition further comprises at least one probe selected from a SARS-CoV-2 E probe, and/or a SARS-CoV-2 N2 probe and/or a SARS CoV-2 RdRP probe. In some embodiments, the SARS-CoV-2 E probe comprises a sequence that is at least 85%, at least 90%, at least 95%, or 100% identical or complementary to at least 15, at least 16, at least 17, at least 18, at least 19, at least 20, at least 21, at least 22, at least 23, at least 24, or at least 25 contiguous nucleotides of SEQ ID NO: 44, and the SARS-CoV-2 N2 probe probe comprises a sequence that is at least 85%, at least 90%, at least 95%, or 100% identical or complementary to at least 15, at least 16, at least 17, at least 18, at least 19, at least 20, at least 21, at least 22, at least 23, at least 24, or at least 25 contiguous nucleotides of SEQ ID NO: 45 or SEQ ID NO: 79. In some embodiments, the SARS-CoV-2 RdRP probe comprises a sequence that is at least 85%, at least 90%, at least 95%, or 100% identical or complementary to at least 15, at least 16, at least 17, at least 18, at least 19, at least 20, at least 21, at least 22, at least 23, at least 24, or at least 25 contiguous nucleotides of SEQ ID NO: 80 and/or SEQ ID NO: 81. In some embodiments, the SARS-CoV-2 E probe comprises a sequence that is at least 85%, at least 90%, at least 95%, or 100% identical or complementary to at least 15, at least 16, at least 17, at least 18, at least 19, at least 20, or at least 21 contiguous nucleotides of SEQ ID NO: 50 or SEQ ID NO: 72, and the SARS-CoV-2 N2 probe comprises a sequence that is at least 85%, at least 90%, at least 95%, or 100% identical or complementary to at least 15, at least 16, at least 17, at least 18, at least 19, at least 20, at least 21, at least 22, at least 23, or at least 24 contiguous nucleotides of SEQ ID NO: 53, SEQ ID NO: 74 and/or SEQ ID NO: 75. In some embodiments, the SARS-CoV-2 RdRP probe comprises a sequence that is at least 85%, at least 90%, at least 95%, or 100% identical or complementary to at least 15, at least 16, at least 17, at least 18, at least 19, at least 20, or at least 21, contiguous nucleotides of SEQ ID NO: 77. In some embodiments, two probes are included for SARS-CoV-2 N2, a first probe that comprises a sequence that is at least 85%, at least 90%, at least 95%, or 100% identical or complementary to at least 15, at least 16, at least 17, at least 18, at least 19, at least 20, at least 21, at least 22, at least 23, or at least 24 contiguous nucleotides of SEQ ID NO: 53 or SEQ ID NO: 74, and a second probe that comprises a sequence that is at least 85%, at least 90%, at least 95%, or 100% identical or complementary to at least 15, at least 16, at least 17, at least 18, at least 19, at least 20, at least 21, at least 22, at least 23, or at least 24 contiguous nucleotides of SEQ ID NO: 75..

In some embodiments, the composition further comprises a probe for detecting an exogenous control.

In some embodiments, each probe comprises a detectable label. In some embodiments, each probe comprises a fluorescent dye and a quencher molecule. In some embodiments, each probe consists of 15 to 30 nucleotides.

In some embodiments, the composition is a lyophilized composition. In some embodiments, the composition is in solution. In some embodiments, the composition comprises nucleic acids from a sample from a subject being tested for the presence or absence of COVID-19, influenza, and/or RSV. In some embodiments, the sample is selected from a nasopharyngeal swab sample, a nasal aspirate sample, a saliva sample and a nasal wash sample.

In some embodiments, kits are provided. In some embodiments, a kit comprises a composition described herein. In some embodiments, the kit further comprises an exogenous control. In some embodiments, the exogenous control is an RNA control. In some embodiments, the RNA control is packaged in a bacteriophage protective coat (e.g., ARMORED® RNA). In some embodiments, the kit comprises dNTPs and/or a thermostable polymerase. In some embodiments, the kit comprises a reverse transcriptase.

In some embodiments, an oligonucleotide comprising a sequence selected from SEQ ID NOs: 17 to 28, 32 to 43, 48 to 65, and 67 to 78 is provided. In some embodiments, the oligonucleotide comprises at least one modified nucleotide. In some embodiments, the oligonucleotide comprises a detectable label. In some embodiments, the oligonucleotide comprises a fluorescent dye and a quencher molecule. In some embodiments, the oligonucleotide is a fluorescence resonance energy transfer (FRET) probe.

### 5. DETAILED DESCRIPTION

5.1. Definitions

To facilitate an understanding of the present invention, a number of terms and phrases are defined below:

As used herein, the terms “detect”, “detecting” or “detection” may describe either the general act of discovering or discerning or the specific observation of a detectably labeled composition.

As used herein, the term “detectably different” refers to a set of labels (such as dyes) that can be detected and distinguished simultaneously.

As used herein, the terms “patient” and “subject” are used interchangeably to refer to a human. In some embodiments, the methods described herein may be used on samples from non-human animals.

As used herein, the terms “oligonucleotide,” “polynucleotide,” “nucleic acid molecule,” and the like, refer to nucleic acid-containing molecules, including but not limited to, DNA or RNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

As used herein, the term “oligonucleotide,” refers to a single-stranded polynucleotide having fewer than 500 nucleotides. In some embodiments, an oligonucleotide is 8 to 200, 8 to 100, 12 to 200, 12 to 100, 12 to 75, or 12 to 50 nucleotides long. Oligonucleotides may be referred to by their length, for example, a 24 residue oligonucleotide may be referred to as a “24-mer.”

As used herein, the term “complementary” to a target RNA (or target region thereof), and the percentage of “complementarity” of the probe sequence to that of the target RNA sequence is the percentage “identity” to the sequence of target RNA or to the reverse complement of the sequence of the target RNA. In determining the degree of “complementarity” between probes used in the compositions described herein (or regions thereof) and a target RNA, such as those disclosed herein, the degree of “complementarity” is expressed as the percentage identity between the sequence of the probe (or region thereof) and sequence of the target RNA or the reverse complement of the sequence of the target RNA that best aligns therewith. The percentage is calculated by counting the number of aligned bases that are identical as between the 2 sequences, dividing by the total number of contiguous nucleotides in the probe, and multiplying by 100. When the term “complementary” is used, the subject oligonucleotide is at least 90% complementary to the target molecule, unless indicated otherwise. In some embodiments, the subject oligonucleotide is at least 91%, at least 92%, at least 93%, at least 94%, at least 95%, at least 96%, at least 97%, at least 98%, at least 99%, or 100% complementary to the target molecule.

A “primer” or “probe” as used herein, refers to an oligonucleotide that comprises a region that is complementary to a sequence of at least 8 contiguous nucleotides of a target nucleic acid molecule, such as DNA (e.g., a target gene) or an mRNA (or a DNA reverse-transcribed from an mRNA). In some embodiments, a primer or probe comprises a region of at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 contiguous nucleotides that is complementary to a sequence of at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 contiguous nucleotides of a target molecule. When a primer or probe comprises a region that is “complementary to at least x contiguous nucleotides” of a target molecule or region of a target molecule or sequence thereof, the primer or probe is at least 95% complementary to at least x contiguous nucleotides of the target molecule. In some embodiments, the primer or probe is at least 96%, at least 97%, at least 98%, at least 99%, or 100% complementary to the target molecule. In some embodiments, two or more forward primers, reverse primers, and/or probes are used to detect the target.

The term “nucleic acid amplification,” encompasses any means by which at least a part of at least one target nucleic acid is reproduced, typically in a template-dependent manner, including without limitation, a broad range of techniques for amplifying nucleic acid sequences, either linearly or exponentially. Exemplary means for performing an amplifying step include polymerase chain reaction (PCR), ligase chain reaction (LCR), ligase detection reaction (LDR), multiplex ligation-dependent probe amplification (MLPA), ligation followed by Q-replicase amplification, primer extension, strand displacement amplification (SDA), hyperbranched strand displacement amplification, multiple displacement amplification (MDA), nucleic acid strand-based amplification (NASB A), two-step multiplexed amplifications, rolling circle amplification (RCA), and the like, including multiplex versions and combinations thereof, for example but not limited to, OLA/PCR, PCR/OLA, LDR/PCR, PCR/PCR/LDR, PCR/LDR, LCR/PCR, PCR/LCR (also known as combined chain reaction--CCR), digital amplification, and the like. Descriptions of such techniques can be found in, among other sources, Ausbel et al.; PCR Primer: A Laboratory Manual, Diffenbach, Ed., Cold Spring Harbor Press (1995); The Electronic Protocol Book, Chang Bioscience (2002); Msuih et al., J. Clin. Micro. 34:501-07 (1996); The Nucleic Acid Protocols Handbook, R. Rapley, ed., Humana Press, Totowa, N.J. (2002); Abramson et al., Curr Opin Biotechnol. 1993 Feb.;4(1):41-7, U.S. Pat. No. 6,027,998; U.S. Pat. No. 6,605,451, Barany et al., PCT Publication No. WO 97/31256; Wenz et al., PCT Publication No. WO 01/92579; Day et al., Genomics, 29(1): 152-162 (1995), Ehrlich et al., Science 252:1643-50 (1991); Innis et al., PCR Protocols: A Guide to Methods and Applications, Academic Press (1990); Favis et al., Nature Biotechnology 18:561-64 (2000); and Rabenau et al., Infection 28:97-102 (2000); Belgrader, Barany, and Lubin, Development of a Multiplex Ligation Detection Reaction DNA Typing Assay, Sixth International Symposium on Human Identification, 1995 (available on the world wide web at: promega.com/geneticidproc/ussymp6proc/blegrad.html); LCR Kit Instruction Manual, Cat. #200520, Rev. #050002, Stratagene, 2002; Barany, Proc. Natl. Acad. Sci. USA 88:188-93 (1991); Bi and Sambrook, Nucl. Acids Res. 25:2924-2951 (1997); Zirvi et al., Nucl. Acid Res. 27:e40i-viii (1999); Dean et al., Proc Natl Acad Sci USA 99:5261-66 (2002); Barany and Gelfand, Gene 109:1-11 (1991); Walker et al., Nucl. Acid Res. 20:1691-96 (1992); Polstra et al., BMC Inf. Dis. 2:18-(2002); Lage et al., Genome Res. 2003 Feb.;13(2):294-307, and Landegren et al., Science 241:1077-80 (1988), Demidov, V., Expert Rev Mol Diagn. 2002 Nov.;2(6):542-8., Cook et al., J Microbiol Methods. 2003 May;53(2):165-74, Schweitzer et al., Curr Opin Biotechnol. 2001 Feb.;12(1):21-7, U.S. Pat. No. 5,830,711, U.S. Pat. No. 6,027,889, U.S. Pat. No. 5,686,243, PCT Publication No. W00056927A3, and PCT Publication No. W09803673A1.

In some embodiments, amplification comprises at least one cycle of the sequential procedures of: annealing at least one primer with complementary or substantially complementary sequences in at least one target nucleic acid; synthesizing at least one strand of nucleotides in a template-dependent manner using a polymerase; and denaturing the newly-formed nucleic acid duplex to separate the strands. The cycle may or may not be repeated. Amplification can comprise thermocycling or can be performed isothermally.

Unless otherwise indicated, the term “hybridize” is used herein refer to “specific hybridization” which is the binding, duplexing, or hybridizing of a nucleic acid molecule preferentially to a particular nucleotide sequence, in some embodiments, under stringent conditions. The term “stringent conditions” refers to conditions under which a probe will hybridize preferentially to its target sequence, and to a lesser extent to, or not at all to, other sequences. A “stringent hybridization” and “stringent hybridization wash conditions” in the context of nucleic acid hybridization (e.g., as in array, Southern, or Northern hybridization) are sequence-dependent and are different under different environmental parameters. An extensive guide to the hybridization of nucleic acids is found in, e.g., Tijssen (1993) Laboratory Techniques in Biochemistry and Molecular Biology—Hybridization with Nucleic Acid Probes part I, Ch. 2, “Overview of principles of hybridization and the strategy of nucleic acid probe assays,” Elsevier, N.Y. (“Tijssen”). Generally, highly stringent hybridization and wash conditions for filter hybridizations are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Very stringent conditions are selected to be equal to the Tₘ for a particular probe. Dependency of hybridization stringency on buffer composition, temperature, and probe length are well known to those of skill in the art (see, e.g., Sambrook and Russell (2001) Molecular Cloning: A Laboratory Manual (3rd ed.) Vol. 1-3, Cold Spring Harbor Laboratory, Cold Spring Harbor Press, NY).

A “sample,” as used herein, includes various nasal samples, such as nasopharyngeal swab samples, oropharyngeal samples, nasal aspirate samples, nasal, or mid-turbinate swab and nasal wash/aspirate specimens, saliva samples, and other types of human samples, including fecal or urine samples. In some embodiments, a nasal sample comprises a buffer, such as a preservative. Further nonlimiting exemplary samples include nasal swabs, oropharyngeal swabs, throat swabs, bronchoalveolar lavage samples, bronchial aspirates, bronchial washes, endotracheal aspirates, endotracheal washes, tracheal aspirates, nasal secretion samples, mucus samples, sputum samples, and lung tissue samples. In some embodiments, the sample comprises a buffer, such as a preservative. In some embodiments the sample is a pooled sample containing sample from two or more subjects.

An “endogenous control,” as used herein refers to a moiety that is naturally present in the sample to be used for detection. In some embodiments, an endogenous control is a “sample adequacy control” (SAC), which may be used to determine whether there was sufficient sample used in the assay, or whether the sample comprised sufficient biological material, such as cells. In some embodiments, an endogenous control is an RNA (such as an mRNA, tRNA, ribosomal RNA, etc.), such as a human RNA. Nonlimiting exemplary endogenous controls include ABL mRNA, GUSB mRNA, GAPDH mRNA, TUBB mRNA, and UPK1a mRNA. In some embodiments, an endogenous control, such as an SAC, is selected that can be detected in the same manner as the target RNA is detected and, in some embodiments, simultaneously with the target RNA.

An “exogenous control,” as used herein, refers to a moiety that is added to a sample or to an assay, such as a “sample processing control” (SPC). In some embodiments, an exogenous control is included with the assay reagents. An exogenous control is typically selected that is not expected to be present in the sample to be used for detection, or is present at very low levels in the sample such that the amount of the moiety naturally present in the sample is either undetectable or is detectable at a much lower level than the amount added to the sample as an exogenous control. In some embodiments, an exogenous control comprises a nucleotide sequence that is not expected to be present in the sample type used for detection of the target RNA. In some embodiments, an exogenous control comprises a nucleotide sequence that is not known to be present in the species from whom the sample is taken. In some embodiments, an exogenous control comprises a nucleotide sequence from a different species than the subject from whom the sample was taken. In some embodiments, an exogenous control comprises a nucleotide sequence that is not known to be present in any species. In some embodiments, an exogenous control is selected that can be detected in the same manner as the target RNA is detected and, in some embodiments, simultaneously with the target RNA. In some embodiments, the exogenous control is an RNA. In some such embodiments, the exogenous control is an ARMORED® RNA, which comprises RNA packaged in a bacteriophage protective coat. See, e.g., WalkerPeach et al., Clin. Chem. 45:12: 2079-2085 (1999).

In the sequences herein, “U” and “T” are used interchangeably, such that both letters indicate a uracil or thymine at that position. One skilled in the art will understand from the context and/or intended use whether a uracil or thymine is intended and/or should be used at that position in the sequence. For example, one skilled in the art would understand that native RNA molecules typically include uracil, while native DNA molecules typically include thymine. Thus, where an RNA sequence includes “T”, one skilled in the art would understand that that position in the native RNA is likely a uracil.

In the present disclosure, “a sequence selected from” encompasses both “one sequence selected from” and “one or more sequences selected from.” Thus, when “a sequence selected from” is used, it is to be understood that one, or more than one, of the listed sequences may be chosen.

In the present disclosure, “SARS-CoV-2” refers to Severe Acute Respiratory Syndrome Coronavirus 2, a novel coronavirus (2019-nCoV) identified in 2019. In the present disclosure, “COVID-19” refers to the disease caused by the SARS-CoV-2 coronavirus. In the present disclosure, “SARS-CoV-2 E” refers to the SARS-CoV-2 envelope protein (E) gene and “SARS-CoV-2 N2” refers to the N2 region in the SARS-CoV-2 nucleoprotein gene. The SARS-CoV-2 E gene and SARS-CoV-2 N2 gene have been identified as markers for detecting SARS-CoV-2 in a sample from a patient. Other portions of the SARS-CoV-2 genome, including but not limited to portions relating to the RNA-dependent RNA polymerase (RdRP) gene of the ORF lab sequence, and other ORF lab genes could also function as markers.

5.2. Detecting Influenza A, Influenza B, RSV, and SARS-CoV-2

The present inventors have developed a combination assay for detecting Influenza A, Influenza B, RSV, and SARS-CoV-2. In some embodiments the assay has at least 95% accuracy in detecting the presence of Influenza A, Influenza B, RSV, and SARS-CoV-2 and at least 95% accuracy in detecting the absence of Influenza A, Influenza B, RSV, and SARS-CoV-2.

In some embodiments, the assay comprises detecting the presence or absence of Flu A polymerase basic 2 (PB2) gene, Flu A polymerase acidic (PA) gene in a sample from a subject, and the Flu A matrix protein (MP) gene. In some embodiments, the assay comprises detecting the influenza A PB2, PA, MP and/or avian MP genes using the forward and reverse primers as shown in Table A. In some embodiments, a probe is used to detect the influenza A PB2, PA, MP and/or avian MP genes, which selectively hybridizes to an amplicon produced by the forward and reverse primers. Nonlimiting exemplary primers and probes for detecting the presence or absence of influenza A are shown in Table A.

In some embodiments, the assay comprises detecting the presence or absence of influenza B MP gene and/or the influenza B NS gene in a sample from a subject. In some embodiments, the assay comprises detecting the influenza B MP and/or NS genes using the forward and reverse primers as shown in Table A. In some embodiments, a probe is used to detect the influenza B MP and/or NS genes, which selectively hybridizes to an amplicon produced by the forward and reverse primers. Nonlimiting exemplary primers and probes for detecting the presence or absence of influenza B are shown in Table A.

In some embodiments, the assay comprises detecting the presence or absence of RSV A and/or RSV B in a sample from a subject. In some embodiments, the assay comprises detecting RSV A and/or RSV B using the forward and reverse primers as shown in Table A. In some embodiments, a probe is used to detect RSV A and/or RSV B, which selectively hybridizes to an amplicon produced by the forward and reverse primers. In some embodiments, two or more forward primers and/or two or more reverse primers are used to detect the genes. Nonlimiting exemplary primers and probes for detecting the presence or absence of RSV are shown in Table A.

In some embodiments, the assay comprises detecting the presence or absence of the SARS-CoV-2 E gene and/or SARS-CoV-2 N2 gene and/or SARS-CoV-2 RdRP gene in a sample from a subject. In some embodiments, the assay comprises detecting the SARS-CoV-2 E gene and/or the SARS-CoV-2 N2 gene and/or SARS CoV-2 RdRP gene using the forward and reverse primers as shown in Table A. In some embodiments, a probe is used to detect the SARS-CoV-2 E gene and/or the SARS-CoV-2 N2 gene and/or SARS CoV-2 RdRP gene, which selectively hybridizes to an amplicon produced by the forward and reverse primers. In some embodiments, two or more probes are used to detect the gene. In some embodiments, two or more forward primers and/or two or more reverse primers are used to detect the gene. Nonlimiting exemplary primers and probes for detecting the presence or absence of SARS-CoV-2 are shown in Table A.

The present assay relies on the polymerase chain reaction (PCR), and can be carried out in a substantially automated manner using a commercially available nucleic acid amplification system. Exemplary nonlimiting nucleic acid amplification systems that can be used to carry out the methods of the invention include the GENEXPERT® system, a GENEXPERT® Infinity system, and GENEXPERT® Xpress System (Cepheid, Sunnyvale, Calif.). In some embodiments, the amplification system may be available at the same location as the individual to be tested, such as a health care provider's office, a clinic, or a community hospital, so processing is not delayed by transporting the sample to another facility. The present assay can be completed in under 3 hours, in some embodiments, under 2 hours, in some embodiments, under 1 hour, in some embodiments, under 45 minutes, in some embodiments, under 35 minutes, and in some embodiments, under 30 minutes, using an automated system, for example, the GENEXPERT® system.

5.2.1. General Methods

Compositions and methods for detecting influenza A, influenza B, RSV, and SARS-CoV-2 are provided. In some embodiments, the method comprises detecting the influenza A PB2 gene, influenza A PA gene, influenza A MP gene, and/or avian MP gene. In some embodiments, the method comprises detecting the influenza B MP gene and/or influenza B NS gene. In some embodiments, the method comprises detecting RSV A and/or RSV B. In some embodiments, the method comprises detecting the SARS-CoV-2 E gene and/or SARS-CoV-2 N2 gene and/or SARS CoV-2 RdRP gene. In some embodiments, the method comprises detecting one or more of SARS-CoV-2, Flu A, avian Flu, Flu B, and RSV.

In some embodiments, a method of detecting Flu A, Flu B, RSV, and/or SARS-CoV-2 in a subject comprises detecting the presence or absence of one or more of SARS-CoV-2, Flu A, avian Flu, Flu B, and RSV genes in a sample from the subject. In some embodiments, the sample is selected from a nasopharyngeal swab sample, a nasal aspirate sample, and a nasal wash sample.

In some embodiments, a method of detecting Flu A, Flu B, RSV, and/or SARS-CoV-2 in a subject further comprises detecting at least one endogenous control, such as a sample adequacy control (SAC). In some embodiments, a method of detecting Flu A, Flu B, RSV, and/or SARS-CoV-2 in a subject further comprises detecting at least one exogenous control, such as a sample processing control (SPC). In some embodiments, the SPC is a RNA control. In some embodiments, the SPC is ARMORED® RNA.

In the present disclosure, the terms “target RNA” and “target gene” are used interchangeably to refer any of the Flu A, Flu B, RSV, avian, and/or SARS-CoV-2 genes described herein, as well as to exogenous and/or endogenous controls. Thus, it is to be understood that when a discussion is presented in terms of a target gene, that discussion is specifically intended to encompass the Flu A, Flu B, RSV, avian, and/or SARS-CoV-2 genes, any endogenous control(s) (e.g., SAC), and any exogenous control(s) (e.g., SPC).

In some embodiments, the presence of the Flu A, Flu B, RSV, avian, and/or SARS-CoV-2 genes is detected in a nasal sample. In some embodiments, the target gene is detected in a nasal aspirate sample or a nasal wash sample. In some embodiments, a target gene is detected in a sample to which a buffer (such as a preservative) has been added. In some embodiments, the presence of the Flu A, Flu B, RSV, avian, and/or SARS-CoV-2 genes is detected in a nasopharyngeal swab sample. In some embodiments, the target gene is detected in an nasopharyngeal swab sample that has been placed in a buffer (such as a preservative).

In some embodiments, detection of the influenza A PB2, PA, MP, and/or avian MP genes in a sample from a subject indicates the presence of Flu A in the subject. In some embodiments, detection of the influenza B MP and/or NS genes in a sample from a subject indicates the presence of Flu B in the subject. In some embodiments, detection of RSV A and/or RSV B in a sample from the subject indicates the presence of RSV in the subject. In some embodiments, detection of the SARS-CoV-2 E and/or SARS-CoV-2 N2 and/or SARS CoV-2 RdRP genes in a sample from the subject indicates the presence of SARS-CoV-2 in the subject.

In some embodiments, the sequence of the influenza A PA gene is at least 95% identical to SEQ ID NO: 2. In some embodiments, the sequence of the influenza A PB2 gene is at least 95% identical to SEQ ID NO: 1. In some embodiments, the sequence of the influenza A MP gene is at least 95% identical to SEQ ID NO: 3. In some embodiments, the sequence of the avian influenza MP gene is at least 95% identical to SEQ ID NO: 4. In some embodiments, the sequence of the influenza B MP gene is at least 95% identical to SEQ ID NO: 6. In some embodiments, the sequence of the influenza B NS gene is at least 95% identical to SEQ ID NO: 7. In some embodiments, the sequence of the SARS-CoV-2 E gene is at least 95% identical to SEQ ID NO: 44. In some embodiments, the sequence of the SARS-CoV-2 N2 gene is at least 95% identical to SEQ ID NO: 45.

In some embodiments, the detecting is done quantitatively. In other embodiments, the detecting is done qualitatively. In some embodiments, detecting a target gene comprises forming a complex comprising a polynucleotide and a nucleic acid selected from a target gene, a cDNA reverse transcribed from a target gene, a DNA amplicon of a target gene, and a complement of a target gene. In some embodiments, detecting a target gene comprises RT-PCR (reverse transcriptase-PCR). In some embodiments, detecting a target gene comprises quantitative RT-PCR or real-time RT-PCR. In some embodiments, a sample adequacy control (SAC) and/or a sample processing control (SPC) is detected in the same assay as the target gene. In some embodiments, if the Flu A, Flu B, RSV, avian, and/or SARS-CoV-2 genes are detected, they are considered to be detected even if the SPC is not detected in the assay. In some embodiments, if the Flu A, Flu B, RSV, avian, and/or SARS-CoV-2 genes are not detected, they are considered to be not detected only if the SPC is detected in the assay.

In some embodiments, the presence of the Flu A, Flu B, RSV, avian, and/or SARS-CoV-2 genes can be measured in samples collected at one or more times from a subject to monitor treatment for Flu, RSV, or COVID-19 in the subject. In some embodiments, the assay may be used in a subject suspected of respiratory tract infection, e.g., after consultation with their healthcare provider. In some embodiments, the present assay may be used as part of routine and/or preventative healthcare for a subject. In some embodiments, the present assay may be used seasonally as part of routine and/or preventative healthcare for a subject. In some embodiments, the present assay may be used as part of routine and/or preventative healthcare for subjects who are at particular risk from Flu, RSV, or COVID-19, such as immunocompromised and elderly subjects.

In some embodiments, a sample to be tested is a nasal aspirate sample or nasal wash sample, or is derived from a nasal aspirate sample or nasal wash sample. In some embodiments, a buffer (such as a preservative) is added to the nasal aspirate sample or nasal wash sample. In some embodiments, the buffer is added to the nasal aspirate sample or nasal wash sample 5 minutes, within 10 minutes, within 30 minutes, within 1 hour, or within 2 hours of sample collection.

In some embodiments, a sample to be tested is a nasopharyngeal swab sample. In some embodiments, the swab is placed in a buffer. In some embodiments, the swab is immediately placed in the buffer. In some embodiments, the swab is placed in the buffer within 5 minutes, within 10 minutes, within 30 minutes, within 1 hour, or within 2 hours of sample collection.

In some embodiments, less than 5 ml, less than 4 ml, less than 3 ml, less than 2 ml, less than 1 ml, or less than 0.75 ml of sample or buffered sample are used in the present methods. In some embodiments, 0.1 ml to 1 ml of sample or buffered sample is used in the present methods.

In some embodiments, the sample to be tested is another bodily fluid, such as saliva, nasal swabs, oropharyngeal swabs, throat swabs, nasal aspirate samples, nasal, or mid-turbinate swab and nasal wash/aspirate samples, bronchoalveolar lavage samples, bronchial aspirates, bronchial washes, endotracheal aspirates, endotracheal washes, tracheal aspirates, nasal secretion samples, mucus samples, sputum samples, lung tissue samples, etc. In some embodiments, the sample to be tested is from other types of human samples, including fecal or urine samples.

The clinical sample to be tested is, in some embodiments, fresh (i.e., never frozen). In other embodiments, the sample is a frozen specimen. In some embodiments, the sample is a tissue sample, such as a formalin-fixed paraffin embedded sample. In some embodiments, the sample is a liquid cytology sample.

In some embodiments, the sample to be tested is obtained from an individual who has one or more symptoms of influenza, RSV, or COVID-19 infection. Nonlimiting exemplary symptoms of influenza include fever, chills, cough, sore throat, runny nose, nasal congestion, muscle ache, headache, fatigue, vomiting, diarrhea, and combinations of any of those symptoms. In some embodiments, the individual may have one or more symptoms that are common between an influenza and COVID-19 infection such as fever, chills, cough, shortness of breath or difficulty breathing, fatigue, sore throat, runny or stuffy nose, muscle or body aches, headache, vomiting, and diarrhea, making it difficult for a health care provider to differentiate between influenza and COVID-19 based on the symptoms. In other embodiments, the individual may have symptoms characteristic of COVID-19, but not typically observed with an influenza or RSV infection, such as loss of smell or taste. In some embodiments, the sample to be tested is obtained from an individual who has previously been diagnosed with influenza, RSV, or COVID-19. In some such embodiments, the individual is monitored for recurrence of influenza, RSV, or COVID-19.

In some embodiments, methods described herein can be used for routine screening of healthy individuals with no risk factors. In some embodiments, methods described herein are used to screen asymptomatic individuals, for example, during routine or preventative care. In some embodiments, methods described herein are used to screen women who are pregnant or who are attempting to become pregnant.

In some embodiments, the methods described herein can be used to assess the effectiveness of a treatment for influenza, RSV, or COVID-19 infection in a patient.

In some embodiments, use of the polymerase acidic (PA) gene, the polymerase basic 2 (PB2) gene, and/or the matrix protein (MP) gene for detecting Flu A is provided. In some embodiments, use of the MP and/or NS gene for detecting Flu B is provided. In some embodiments, use of the RSV A and/or RSV B gene for detecting RSV is provided. In some embodiments, use of the SARS-CoV-2E and/or SARS-CoV-2 N2 gene and/or SARS CoV-2 RdRP gene for detecting SARS-CoV-2 is provided. In some embodiments, use of an avian MP influenza gene to detect avian flu is also provided.

In any of the embodiments described herein, the Flu A, Flu B, RSV, avian, and/or SARS-CoV-2 genes may be detected in the same assay reaction as a sample processing control (SPC).

In some embodiments, a method of facilitating detection of Flu A, Flu B, RSV, avian, and/or SARS-CoV-2 genes infection in a subject is provided. Such methods comprise detecting the presence or absence of the Flu A PB2 gene, Flu A PA gene, and/or Flu A MP gene in a sample from the subject; detecting the presence or absence of the Flu B MP and/or Flu B NS gene in a sample from the subject; determining the presence or absence of the RSV A and/or RSV B gene in a sample from the subject; determining the presence or absence of the SARS-CoV-2E and/or SARS-CoV-2 N2 and/or SARS CoV-2 RdRP gene in a sample from the subject; and/or detecting the presence or absence of an avian flu gene in a sample from the subject. In some embodiments, information concerning the presence or absence of the Flu A, Flu B, RSV, avian, and/or SARS-CoV-2 genes in the sample from the subject is communicated to a medical practitioner. A “medical practitioner,” as used herein, refers to an individual or entity that diagnoses and/or treats patients, such as a hospital, a clinic, a physician's office, a physician, a nurse, or an agent of any of the aforementioned entities and individuals. In some embodiments, detecting the presence or absence of Flu A, Flu B, RSV, avian, and/or SARS-CoV-2 genesis carried out at a laboratory that has received the subject's sample from the medical practitioner or agent of the medical practitioner. The laboratory carries out the detection by any method, including those described herein, and then communicates the results to the medical practitioner. A result is “communicated,” as used herein, when it is provided by any means to the medical practitioner. In some embodiments, such communication may be oral or written, may be by telephone, in person, by e-mail, by mail or other courier, or may be made by directly depositing the information into, e.g., a database accessible by the medical practitioner, including databases not controlled by the medical practitioner. In some embodiments, the information is maintained in electronic form. In some embodiments, the information can be stored in a memory or other computer readable medium, such as RAM, ROM, EEPROM, flash memory, computer chips, digital video discs (DVD), compact discs (CDs), hard disk drives (HDD), magnetic tape, etc.

In some embodiments, methods of detecting Flu A, Flu B, RSV, avian, and/or SARS-CoV-2 are provided. In some embodiments, methods of diagnosing Flu A, Flu B, RSV, avian, and/or SARS-CoV-2 infection are provided. In some embodiments, the method comprises obtaining a sample from a subject and providing the sample to a laboratory for detection of the Flu A, Flu B, RSV, avian, and/or SARS-CoV-2 genes in the sample. In some embodiments, the method further comprises receiving a communication from the laboratory that indicates the presence or absence of the Flu A, Flu B, RSV, avian, and/or SARS-CoV-2 genes in the sample. A “laboratory,” as used herein, is any facility that detects the target gene in a sample by any method, including the methods described herein, and communicates the result to a medical practitioner. In some embodiments, a laboratory is under the control of a medical practitioner. In some embodiments, a laboratory is not under the control of the medical practitioner.

When a laboratory communicates the result of detecting the presence or absence of the Flu A, Flu B, RSV, avian, and/or SARS-CoV-2genes to a medical practitioner, in some embodiments, the laboratory indicates whether or not the Flu A, Flu B, RSV, avian, and/or SARS-CoV-2genes were detected in the sample.

As used herein, when a method relates to detecting Flu A, Flu B, RSV, avian, and/or SARS-CoV-2; determining the presence of Flu A, Flu B, RSV, avian, and/or SARS-CoV-2; monitoring for Flu A, Flu B, RSV, avian, and/or SARS-CoV-2; and/or diagnosing Flu A, Flu B, RSV, avian, and/or SARS-CoV-2 infection, the method includes activities in which the steps of the method are carried out, but the result is negative for the presence of Flu A, Flu B, RSV, avian, and/or SARS-CoV-2. That is, detecting, determining, monitoring, and diagnosing Flu A, Flu B, RSV, avian, and/or SARS-CoV-2 infection include instances of carrying out the methods that result in either positive or negative results.

In some embodiments, at least one endogenous control (e.g., an SAC) and/or at least one exogenous control (e.g., an SPC) are detected simultaneously with the Flu A, Flu B, RSV, avian, and/or SARS-CoV-2 genes in a single reaction. In some embodiments, at least one exogenous control (e.g., an SPC) is detected simultaneously with the Flu A, Flu B, RSV, avian, and/or SARS-CoV-2 genes in a single reaction.

5.2.2. Exemplary Controls

In some embodiments, an assay described herein comprises detecting the Flu A, Flu B, RSV, avian, and/or SARS-CoV-2 genes and at least one endogenous control. In some embodiments, the endogenous control is a sample adequacy control (SAC). In some such embodiments, if none of the Flu A, Flu B, RSV, avian, and/or SARS-CoV-2 genes are is in a sample, and the SAC is also not detected in the sample, the assay result is considered “invalid” because the sample may have been insufficient. While not intending to be bound by any particular theory, an insufficient sample may be too dilute, contain too little cellular material, contain an assay inhibitor, etc. In some embodiments, the failure to detect an SAC may indicate that the assay reaction failed. In some embodiments, an endogenous control is an RNA (such as an mRNA, tRNA, ribosomal RNA, etc.). Nonlimiting exemplary endogenous controls include ABL mRNA, GUSB mRNA, GAPDH mRNA, TUBB mRNA, and UPK1a mRNA.

In some embodiments, an assay described herein comprises detecting the Flu A, Flu B, RSV, avian, and/or SARS-CoV-2 genes and at least one exogenous control. In some embodiments, the exogenous control is a sample processing control (SPC). In some such embodiments, for example, if the PB2 gene and/or the PA gene is not detected in a sample, and the SPC is also not detected in the sample, the assay result is considered “invalid” because there may have been an error in sample processing, including but not limited to, failure of the assay. Nonlimiting exemplary errors in sample processing include, inadequate sample processing, the presence of an assay inhibitor, the presence of a nuclease (such as an RNase), compromised reagents, etc. In some embodiments, an exogenous control (such as an SPC) is added to a sample. In some embodiments, an exogenous control (such as an SPC) is added during performance of an assay, such as with one or more buffers or reagents. In some embodiments, when a GENEXPERT® system is to be used, the SPC is included in the GENEXPERT® cartridge. In some embodiments, an exogenous control (such as an SPC) is an ARMORED® RNA, which is protected by a bacteriophage coat.

In some embodiments, an endogenous control and/or an exogenous control is detected contemporaneously, such as in the same assay, as detection of the Flu A, Flu B, RSV, avian, and/or SARS-CoV-2 genes. In some embodiments, an assay comprises reagents for detecting the Flu A, Flu B, RSV, avian, and/or SARS-CoV-2 genes and an exogenous control simultaneously in the same assay reaction. In some such embodiments, for example, an assay reaction comprises a primer set for amplifying each of the Flu A, Flu B, RSV, avian, and/or SARS-CoV-2 genes, and a primer set for amplifying an exogenous control, and labeled probes for detecting the amplification products (such as, for example, TAQMAN® probes).

5.2.3. Exemplary Sample Preparation

5.2.3.1. Exemplary Buffers

In some embodiments, a buffer is added to the sample. In some embodiments, the buffer is added within one hour, two hours, three hours, or six hours of the time the sample was collected. In some embodiments, a buffer is added to the sample within one hour, two hours, three hours, or six hours before the sample is analyzed by the methods described herein.

In some embodiments, a swab sample is placed in a buffer. In some embodiments, the swab sample is placed in the buffer within one hour, two hours, three hours, or six hours of the time the swab sample was collected. In some embodiments, the swab sample is placed in a buffer within one hour, two hours, three hours, or six hours before the sample is analyzed by the methods described herein.

Non-limiting exemplary commercial buffers include the viral transport medium provided with the GENEXPERT® Nasal Pharyngeal Collection Kit (Cepheid, Sunnyvale, Calif.); specimen collection and transport device optimized for molecular assays (e-NAT™, Copan, Murrieta, Calif.); universal transport medium (UTMTM, Copan, Murrieta, Calif.); universal viral transport medium (UVT, BD, Franklin Lakes, N.J.); M4, M4RT, M5, and M6 (Thermo Scientific). Further nonlimiting exemplary buffers include liquid Amies medium, PBS/0.5% BSA, PBS/0.5% gelatin, Bartel BiraTrans™ medium, EMEM, PBS, EMEM/1% BSA, sucrose phosphate, Trypticase™ soy broth (with or without 0.5% gelatin or 0.5% BSA), modified Stuart's medium, veal infusion broth (with or without 0.5% BSA), and saline. Buffers that contain a chaotropic agent such as guanidine thiocyanate, guanidine hydrochloride, and guanidine iosthionate may also be used. These buffers may optionally contain one or more detergents, one or more reducing agents and one or more chelators.

5.2.3.2. Exemplary RNA Preparation

Target RNA can be prepared by any appropriate method. Total RNA can be isolated by any method, including, but not limited to, the protocols set forth in Wilkinson, M. (1988) Nucl. Acids Res. 16(22):10,933; and Wilkinson, M. (1988) Nucl. Acids Res. 16(22): 10934, or by using commercially-available kits or reagents, such as the TRIzol® reagent (Invitrogen), Total RNA Extraction Kit (iNtRON Biotechnology), Total RNA Purification Kit (Norgen Biotek Corp.), RNAqueous™ (Invitrogen), MagMAX™ (Applied Biosystems), RecoverAll™ (Invitrogen), RNAeasy (Qiagen), etc.

In some embodiments, RNA levels are measured in a sample in which RNA has not first been purified from the cells. In some such embodiments, the cells are subject to a lysis step to release the RNA. Nonlimiting exemplary lysis methods include sonication (for example, for 2-15 seconds, 8-18 μm at 36 kHz); chemical lysis, for example, using a detergent; and various commercially available lysis reagents (such as RNAeasy lysis buffer, Qiagen). In some embodiments, RNA levels are measured in a sample in which RNA has been isolated.

In some embodiments, RNA is modified before a target RNA is detected. In some embodiments, all of the RNA in the sample is modified. In some embodiments, just the particular target RNAs to be analyzed are modified, e.g., in a sequence-specific manner. In some embodiments, RNA is reverse transcribed. In some such embodiments, RNA is reverse transcribed using a reverse transcriptase enzyme such as MMLV, AMV or variants thereof that have been engineered to have features such as reduced RNAse H activity and increased processivity, sensitivity, and/or thermostability. Nonlimiting exemplary conditions for reverse transcribing RNA using MMLV reverse transcriptase include incubation from 5 to 20 minutes at 40° C. to 50° C.

When a target RNA is reverse transcribed, a DNA complement of the target RNA is formed. In some embodiments, the complement of a target RNA is detected rather than a target RNA itself (or a DNA copy of the RNA itself). Thus, when the methods discussed herein indicate that a target RNA is detected, or the level of a target RNA is determined, such detection or determination may be carried out on a complement of a target RNA instead of, or in addition to, the target RNA itself. In some embodiments, when the complement of a target RNA is detected rather than the target RNA, a polynucleotide for detection is used that is complementary to the complement of the target RNA. In some such embodiments, a polynucleotide for detection comprises at least a portion that is identical in sequence to the target RNA, although it may contain thymidine in place of uridine, and/or comprise other modified nucleotides.

5.2.4. Exemplary Analytical Methods

As described above, methods are presented for detecting SARS-CoV-2, influenza, and respiratory syncytial virus (RSV). In some embodiments, the methods comprise detecting the presence of the Flu A polymerase basic 2 (PB 2) gene, polymerase acidic (PA) gene, Flu A matrix protein (MP) gene, and/or the avian influenza MP gene in a sample from a subject. In some embodiments, the method comprises detecting the presence of the Flu B nonstructural protein (NS) gene and/or Flu B matrix protein (MP) gene. In some embodiments, the method comprises detecting the presence of the RSV A genome and/or RSV B genome. In some embodiments, the method comprises detecting the presence of the SARS-CoV-2 E and/or SARS-CoV-2 N2 and/or SARS-CoV-2 RdRP gene. In some embodiments, the method optionally comprises detecting the presence of at least one exogenous control (such as an SPC). In some embodiments, detection of one or more genes selected from Flu A polymerase basic 2 (PB2) gene, polymerase acidic (PA) gene, Flu A matrix protein (MP) gene, Flu B nonstructural protein (NS) gene, Flu B matrix protein (MP) gene, and avian matrix protein (MP) gene indicates the presence of influenza, even if the endogenous control and/or exogenous control is not detected in the assay. In some embodiments, detection of RSV A or RSV B indicates the presence of RSV, even if the endogenous control and/or exogenous control is not detected in the assay. In some embodiments, detection of SARS-CoV-2 E and/or SARS-CoV-2 N2 and/or SARS-CoV 2 RdRP indicates the presence of SARS-CoV-2, even if the endogenous control and/or exogenous control is not detected in the assay. In some embodiments, if none of the flu A or B target genes (such as the Flu A polymerase basic 2 (PB2) gene, polymerase acidic (PA) gene, Flu A matrix protein (MP) gene, Flu B matrix protein (MP) gene, Flu B nonstructural protein (NS) gene, and avian influenza matrix protein (MP) gene) is detected, the result is considered to be negative for influenza only if the control detected. In some embodiments, if none of the RSV target genes is detected, the result is considered to be negative for RSV only if the control detected. In some embodiments, if none of the SARS-CoV-2 target genes is detected, the result is considered to be negative for SARS-CoV-2 only if the control detected.

In some embodiments, detection of any one, any two or all three of the three target genes, SARS-CoV-2 E or SARS-CoV-2 N2 or SARS-CoV 2 RdRP, in a sample indicates the presence of SARS-CoV-2. Large-scale sustained person-to-person transmission of SARS-CoV-2 has led to many mutational events that have been shown to affect the sensitivity and specificity of PCR assays. Mutations in the binding site for any of the primers or probes used in an assay may result in loss of detection of that target and potentially a false negative result. Mutations have been reported throughout the genome of SARS-CoV-2, including within the E and the N genes (Plante et al. Cell Host Microbe. 2021 Apr. 14; 29(4): 508-515, Ziegler et al. Euro Surveill. 2020;25(39) and Hasan et al. J Clin Microbiol 59:e03278-20). Use of multiple targets and redundant primers and or probes mitigates the impact of any single mutation. If one of the targets is not detected but a second or third target is detected that can be interpreted as a positive or presumptive positive result.

Any analytical procedure capable of permitting specific detection of a target gene may be used in the methods herein presented. Exemplary nonlimiting analytical procedures include, but are not limited to, nucleic acid amplification methods, PCR methods, isothermal amplification methods, and other analytical detection methods known to those skilled in the art.

In some embodiments, the method of detecting a target gene comprises amplifying the gene and/or a complement thereof. Such amplification can be accomplished by any method. Exemplary methods include, but are not limited to, isothermal amplification, real time RT-PCR, endpoint RT-PCR, and amplification using T7 polymerase from a T7 promoter annealed to a DNA, such as provided by the SenseAmp Plus™ Kit available at Implen, Germany.

When a target gene is amplified, in some embodiments, an amplicon of the target gene is formed. An amplicon may be single stranded or double-stranded. In some embodiments, when an amplicon is single-stranded, the sequence of the amplicon is related to the target gene in either the sense or antisense orientation. In some embodiments, an amplicon of a target gene is detected rather than the target gene itself. Thus, when the methods discussed herein indicate that a target gene is detected, such detection may be carried out on an amplicon of the target gene instead of, or in addition to, the target gene itself. In some embodiments, when the amplicon of the target gene is detected rather than the target gene, a polynucleotide for detection is used that is complementary to the complement of the target gene. In some embodiments, when the amplicon of the target gene is detected rather than the target gene, a polynucleotide for detection is used that is complementary to the target gene. Further, in some embodiments, multiple polynucleotides for detection may be used, and some polynucleotides may be complementary to the target gene and some polynucleotides may be complementary to the complement of the target gene.

In some embodiments, the method of detecting a target gene comprises PCR, as described below. In some embodiments, detecting one or more target genes comprises real-time monitoring of a PCR reaction, which can be accomplished by any method. Such methods include, but are not limited to, the use of TAQMAN®, molecular beacons, or Scorpion probes (i.e., energy transfer (ET) probes, such as FRET probes) and the use of intercalating dyes, such as SYBR green, EvaGreen, thiazole orange, YO-PRO, TO-PRO, etc.

Nonlimiting exemplary conditions for amplifying a cDNA that has been reverse transcribed from the target RNA are as follows. An exemplary cycle comprises an initial denaturation at 90° C. to 100° C. for 20 seconds to 5 minutes, followed by cycling that comprises denaturation at 90° C. to 100° C. for 1 to 10 seconds, followed by annealing and amplification at 60° C. to 75° C. for 10 to 40 seconds. A further exemplary cycle comprises 20 seconds at 94° C., followed by up to 3 cycles of 1 second at 95° C., 35 seconds at 62° C., 20 cycles of 1 second at 95° C., 20 seconds at 62° C., and 14 cycles of 1 second at 95° C., 35 seconds at 62° C. In some embodiments, for the first cycle following the initial denaturation step, the cycle denaturation step is omitted. In some embodiments, Taq polymerase is used for amplification. In some embodiments, the cycle is carried out at least 10 times, at least 15 times, at least 20 times, at least 25 times, at least 30 times, at least 35 times, at least 40 times, or at least 45 times. In some embodiments, Taq is used with a hot start function. In some embodiments, the amplification reaction occurs in a GENEXPERT® cartridge, and amplification of the target genes and an exogenous control occurs in the same reaction. In some embodiments, detection of the target genes occurs in less than 3 hours, less than 2.5 hours, less than 2 hours, less than 1 hour, less than 45 minutes, less than 40 minutes, less than 35 minutes, or less than 30 minutes from initial denaturation through the last extension.

In some embodiments, detection of a target gene comprises forming a complex comprising a polynucleotide that is complementary to a target gene or to a complement thereof, and a nucleic acid selected from the target gene, a DNA amplicon of the target gene, and a complement of the target gene. Thus, in some embodiments, the polynucleotide forms a complex with a target gene. In some embodiments, the polynucleotide forms a complex with a complement of the target RNA, such as a cDNA that has been reverse transcribed from the target RNA. In some embodiments, the polynucleotide forms a complex with a DNA amplicon of the target gene. When a double-stranded DNA amplicon is part of a complex, as used herein, the complex may comprise one or both strands of the DNA amplicon. Thus, in some embodiments, a complex comprises only one strand of the DNA amplicon. In some embodiments, a complex is a triplex and comprises the polynucleotide and both strands of the DNA amplicon. In some embodiments, the complex is formed by hybridization between the polynucleotide and the target gene, complement of the target gene, or DNA amplicon of the target gene. The polynucleotide, in some embodiments, is a primer or probe.

In some embodiments, a method comprises detecting the complex. In some embodiments, the complex does not have to be associated at the time of detection. That is, in some embodiments, a complex is formed, the complex is then dissociated or destroyed in some manner, and components from the complex are detected. An example of such a system is a TAQMAN® assay. In some embodiments, when the polynucleotide is a primer, detection of the complex may comprise amplification of the target gene, a complement of the target gene, or a DNA amplicon of the target gene.

In some embodiments the analytical method used for detecting at least one target gene in the methods set forth herein includes real-time quantitative PCR. In some embodiments, the analytical method used for detecting at least one target gene includes the use of a TAQMAN® probe. The assay uses energy transfer (“ET”), such as fluorescence resonance energy transfer (“FRET”), to detect and quantitate the synthesized PCR product. Typically, the TAQMAN® probe comprises a fluorescent dye molecule coupled to the 5′-end and a quencher molecule coupled to the 3′-end, such that the dye and the quencher are in close proximity, allowing the quencher to suppress the fluorescence signal of the dye via FRET. When the polymerase replicates the chimeric amplicon template to which the TAQMAN® probe is bound, the 5′-nuclease of the polymerase cleaves the probe, decoupling the dye and the quencher so that the dye signal (such as fluorescence) is detected. Signal (such as fluorescence) increases with each PCR cycle proportionally to the amount of probe that is cleaved.

In some embodiments, a target gene is considered to be detected if any signal is generated from the TAQMAN® probe during the PCR cycling. For example, in some embodiments, if the PCR includes 40 cycles, if a signal is generated at any cycle during the amplification, the target gene is considered to be present and detected. In some embodiments, if no signal is generated by the end of the PCR cycling, the target gene is considered to be absent and not detected.

In some embodiments, quantitation of the results of real-time PCR assays is done by constructing a standard curve from a nucleic acid of known concentration and then extrapolating quantitative information for target genes of unknown concentration. In some embodiments, the nucleic acid used for generating a standard curve is a DNA (for example, an endogenous control, or an exogenous control). In some embodiments, the nucleic acid used for generating a standard curve is a purified double-stranded plasmid DNA or a single-stranded DNA generated in vitro.

In some embodiments, in order for an assay to indicate that Flu is not present in a sample, the Ct values for an endogenous control (such as an SAC) and/or an exogenous control (such as an SPC) must be within a previously-determined valid range. That is, in some embodiments, the absence of Flu cannot be confirmed unless the controls are detected, indicating that the assay was successful. In some embodiments, the assay includes an exogenous control. Ct values are inversely proportional to the amount of nucleic acid target in a sample.

In some embodiments, a threshold Ct (or a “cutoff Ct”) value for a target gene (including an endogenous control and/or exogenous control), below which the gene is considered to be detected, has previously been determined. In some embodiments, a threshold Ct is determined using substantially the same assay conditions and system (such as a GENEXPERT®) on which the samples will be tested.

In addition to the TAQMAN® assays, other real-time PCR chemistries useful for detecting and quantitating PCR products in the methods presented herein include, but are not limited to, Molecular Beacons, Scorpion probes and intercalating dyes, such as SYBR Green, EvaGreen, thiazole orange, YO-PRO, TO-PRO, etc., which are discussed below.

In various embodiments, real-time PCR detection is utilized to detect, in a single multiplex reaction, the Flu target genes, and optionally, one or more RSV target genes, an endogenous control, and an exogenous control. In some multiplex embodiments, a plurality of probes, such as TAQMAN® probes, each specific for a different target, is used. In some embodiments, each target gene-specific probe is spectrally distinguishable from the other probes used in the same multiplex reaction. A nonlimiting exemplary seven-color multiplex system is described, e.g., in Lee et al., BioTechniques, 27: 342-349.

In some embodiments, quantitation of real-time RT PCR products is accomplished using a dye that binds to double-stranded DNA products, such as SYBR Green, EvaGreen, thiazole orange, YO-PRO, TO-PRO, etc. In some embodiments, the assay is the QuantiTect SYBR® Green PCR assay from Qiagen. In this assay, total RNA is first isolated from a sample. Total RNA is subsequently poly-adenylated at the 3′-end and reverse transcribed using a universal primer with poly-dT at the 5′-end. In some embodiments, a single reverse transcription reaction is sufficient to assay multiple target RNAs. Real-time RT-PCR is then accomplished using target RNA-specific primers and an miScript Universal Primer, which comprises a poly-dT sequence at the 5′-end. SYBR Green dye binds non-specifically to double-stranded DNA and upon excitation, emits light. In some embodiments, buffer conditions that promote highly-specific annealing of primers to the PCR template (e.g., available in the QuantiTect SYBR® Green PCR Kit from Qiagen) can be used to avoid the formation of non-specific DNA duplexes and primer dimers that will bind SYBR Green and negatively affect quantitation. Thus, as PCR product accumulates, the signal from SYBR Green increases, allowing quantitation of specific products.

Real-time PCR is performed using any PCR instrumentation available in the art. Typically, instrumentation used in real-time PCR data collection and analysis comprises a thermal cycler, optics for fluorescence excitation and emission collection, and optionally a computer and data acquisition and analysis software.

In some embodiments, detection and/or quantitation of real-time PCR products is accomplished using a dye that binds to double-stranded DNA products, such as SYBR Green, EvaGreen, thiazole orange, YO-PRO, TO-PRO, etc. In some embodiments, the analytical method used in the methods described herein is a DASL® (DNA-mediated Annealing, Selection, Extension, and Ligation) Assay. In some embodiments, total RNA is isolated from a sample to be analyzed by any method. Total RNA may then be polyadenylated (>18 A residues are added to the 3′-ends of the RNAs in the reaction mixture). The RNA is reverse transcribed using a biotin-labeled DNA primer that comprises from the 5′ to the 3′ end, a sequence that includes a PCR primer site and a poly-dT region that binds to the poly-dA tail of the sample RNA. The resulting biotinylated cDNA transcripts are then hybridized to a solid support via a biotin-streptavidin interaction and contacted with one or more target RNA-specific polynucleotides. The target RNA-specific polynucleotides comprise, from the 5′-end to the 3′-end, a region comprising a PCR primer site, region comprising an address sequence, and a target RNA-specific sequence.

In some DASL® embodiments, the target RNA-specific sequence comprises at least 8, at least 9, at least 10, at least 11, at least 12, at least 13, at least 14, at least 15, at least 16, at least 17, at least 18, at least 19 contiguous nucleotides having a sequence that is the same as, or complementary to, at least 8, at least 9, at least 10, at least 11, at least 12, at least 13, at least 14, at least 15, at least 16, at least 17, at least 18, at least 19 contiguous nucleotides of a target RNA, an endogenous control RNA, or an exogenous control RNA.

After hybridization, the target RNA-specific polynucleotide is extended, and the extended products are then eluted from the immobilized cDNA array. A second PCR reaction using a fluorescently-labeled universal primer generates a fluorescently-labeled DNA comprising the target RNA-specific sequence. The labeled PCR products are then hybridized to a microbead array for detection and quantitation.

In some embodiments, the analytical method used for detecting and quantifying the target genes in the methods described herein is a bead-based flow cytometric assay. See Lu J. et al. (2005) Nature 435:834-838, which is incorporated herein by reference in its entirety. An example of a bead-based flow cytometric assay is the xMAP® technology of Luminex, Inc. See luminexcorp.com. In some embodiments, total RNA is isolated from a sample and is then labeled with biotin. The labeled RNA is then hybridized to target RNA-specific capture probes (e.g., FlexmiR™ products sold by Luminex, Inc. at luminexcorp.com) that are covalently bound to microbeads, each of which is labeled with 2 dyes having different fluorescence intensities. A streptavidin-bound reporter molecule (e.g., streptavidin-phycoerythrin, also known as “SAPE”) is attached to the captured target RNA and the unique signal of each bead is read using flow cytometry. In some embodiments, the RNA sample is first polyadenylated, and is subsequently labeled with a biotinylated 3DNA™ dendrimer (i.e., a multiple-arm DNA with numerous biotin molecules bound thereto), using a bridging polynucleotide that is complementary to the 3′-end of the poly-dA tail of the sample RNA and to the 5′-end of the polynucleotide attached to the biotinylated dendrimer. The streptavidin-bound reporter molecule is then attached to the biotinylated dendrimer before analysis by flow cytometry. In some embodiments, biotin-labeled RNA is first exposed to SAPE, and the RNA/SAPE complex is subsequently exposed to an anti-phycoerythrin antibody attached to a DNA dendrimer, which can be bound to as many as 900 biotin molecules. This allows multiple SAPE molecules to bind to the biotinylated dendrimer through the biotin-streptavidin interaction, thus increasing the signal from the assay.

In some embodiments, the analytical method used for detecting and quantifying the levels of the at least one target gene in the methods described herein is by gel electrophoresis and detection with labeled probes (e.g., probes labeled with a radioactive or chemiluminescent label), such as by northern blotting. In some embodiments, total RNA is isolated from the sample, and then is size-separated by SDS polyacrylamide gel electrophoresis. The separated RNA is then blotted onto a membrane and hybridized to radiolabeled complementary probes. In some embodiments, exemplary probes contain one or more affinity-enhancing nucleotide analogs as discussed below, such as locked nucleic acid (“LNA”) analogs, which contain a bicyclic sugar moiety instead of deoxyribose or ribose sugars. See, e.g., Várallyay, E. et al. (2008) Nature Protocols 3(2):190-196, which is incorporated herein by reference in its entirety.

In some embodiments, detection and quantification of one or more target genes is accomplished using microfluidic devices and single-molecule detection. In some embodiments, target RNAs in a sample of isolated total RNA are hybridized to two probes, one which is complementary to nucleic acids at the 5′-end of the target RNA and the second which is complementary to the 3′-end of the target RNA. Each probe comprises, in some embodiments, one or more affinity-enhancing nucleotide analogs, such as LNA nucleotide analogs and each is labeled with a different fluorescent dye having different fluorescence emission spectra (i.e., detectably different dyes). The sample is then flowed through a microfluidic capillary in which multiple lasers excite the fluorescent probes, such that a unique coincident burst of photons identifies a particular target RNA, and the number of particular unique coincident bursts of photons can be counted to quantify the amount of the target RNA in the sample. In some alternative embodiments, a target RNA-specific probe can be labeled with 3 or more distinct labels selected from, e.g., fluorophores, electron spin labels, etc., and then hybridized to an RNA sample.

Optionally, the sample RNA is modified before hybridization. The target RNA/probe duplex is then passed through channels in a microfluidic device and that comprise detectors that record the unique signal of the 3 labels. In this way, individual molecules are detected by their unique signal and counted. See, e.g., U.S. Pat. Nos. 7,402,422 and 7,351,538 to Fuchs et al., U.S. Genomics, Inc., each of which is incorporated herein by reference in its entirety.

5.2.5. Exemplary Automation and Systems

In some embodiments, gene expression is detected using an automated sample handling and/or analysis platform. In some embodiments, commercially available automated analysis platforms are utilized. For example, in some embodiments, the GENEXPERT® system (Cepheid, Sunnyvale, Calif.) is utilized.

The present invention is illustrated for use with the GENEXPERT® system. Exemplary sample preparation and analysis methods are described below. However, the present invention is not limited to a particular detection method or analysis platform. One of skill in the art recognizes that any number of platforms and methods may be utilized.

The GENEXPERT® utilizes a self-contained, single use cartridge. Sample extraction, amplification, and detection may all carried out within this self-contained “laboratory in a cartridge.” (See, e.g., U.S. Pat. Nos. 5,958,349, 6,403,037, 6,440,725, 6,783,736, 6,818,185; each of which is herein incorporated by reference in its entirety.)

Components of the cartridge include, but are not limited to, processing chambers containing reagents, filters, and capture technologies useful to extract, purify, and amplify target nucleic acids. A valve enables fluid transfer from chamber to chamber and contain nucleic acids lysis and filtration components. An optical window enables real-time optical detection. A reaction tube enables very rapid thermal cycling.

In some embodiments, the GENEXPERT® system includes a plurality of modules for scalability. Each module includes a plurality of cartridges, along with sample handling and analysis components.

After the sample is added to the cartridge, the sample is contacted with lysis buffer and released nucleic acid (NA) is bound to an NA-binding substrate such as a silica or glass substrate. The sample supernatant is then removed and the NA eluted in an elution buffer such as a Tris/EDTA buffer. The eluate may then be processed in the cartridge to detect target genes as described herein. In some embodiments, the eluate is used to reconstitute at least some of the PCR reagents, which are present in the cartridge as lyophilized particles.

In some embodiments, RT-PCR is used to amplify and analyze the presence of the target genes. In some embodiments, the reverse transcription uses MMLV RT enzyme and an incubation of 5 to 20 minutes at 40° C. to 50° C. In some embodiments, the PCR uses Taq polymerase with hot start function, such as AptaTaq (Roche). In some embodiments, the initial denaturation is at 90° C. to 100° C. for 20 seconds to 5 minutes; the cycling denaturation temperature is 90° C. to 100° C. for 1 to 10 seconds; the cycling anneal and amplification temperature is 60° C. to 75° C. for 10 to 40 seconds; and up to 50 cycles are performed.

In some embodiments, a double-denature method is used to amplify low copy number targets. A double-denature method comprises, in some embodiments, a first denaturation step followed by addition of primers and/or probes for detecting target genes. All or a substantial portion of the nucleic acid-containing sample (such as a DNA eluate) is then denatured a second time before, in some instances, a portion of the sample is aliquotted for cycling and detection of the target genes. While not intending to be bound by any particular theory, the double-denature protocol may increase the chances that a low copy number target gene (or its complement) will be present in the aliquot selected for cycling and detection because the second denaturation effectively doubles the number of targets (i.e., it separates the target and its complement into two separate templates) before an aliquot is selected for cycling. In some embodiments, the first denaturation step comprises heating to a temperature of 90° C. to 100° C. for a total time of 30 seconds to 5 minutes. In some embodiments, the second denaturation step comprises heating to a temperature of 90° C. to 100° C. for a total time of 5 seconds to 3 minutes. In some embodiments, the first denaturation step and/or the second denaturation step is carried out by heating aliquots of the sample separately. In some embodiments, each aliquot may be heated for the times listed above. As a non-limiting example, a first denaturation step for an NA-containing sample (such as a DNA eluate) may comprise heating at least one, at least two, at least three, or at least four aliquots of the sample separately (either sequentially or simultaneously) to a temperature of 90° C. to 100° C. for 60 seconds each. As a non-limiting example, a second denaturation step for a NA-containing sample (such as a DNA eluate) containing enzyme, primers, and probes may comprise heating at least one, at least two, at least three, or at least four aliquots of the eluate separately (either sequentially or simultaneously) to a temperature of 90° C. to 100° C. for 5 seconds each. In some embodiments, an aliquot is the entire NA-containing sample (such as a DNA eluate). In some embodiments, an aliquot is less than the entire NA-containing sample (such as a DNA eluate).

In some embodiments, target genes in a NA-containing sample, such as a DNA eluate, are detected using the following protocol: One or more aliquots of the NA-containing sample are heated separately to 95° C. for 60 seconds each. The enzyme and primers and probes are added to the NA-containing sample and one or more aliquots are heated separately to 95° C. for 5 seconds each. At least one aliquot of the NA-containing sample containing enzyme, primers, and probes is then heated to 94° C. for 60 seconds. The aliquot is then cycled 45 times with the following 2-step cycle: (1) 94° C. for 5 seconds, (2) 66° C. for 30 seconds.

The present invention is not limited to particular primer and/or probe sequences. Exemplary amplification primers and detection probes are described in the Examples and are shown in Table A.

In some embodiments, an off-line centrifugation is used, for example, with samples with low cellular content. The sample, with or without a buffer added, is centrifuged and the supernatant removed. The pellet is then resuspended in a smaller volume of either supernatant or the buffer. The resuspended pellet is then analyzed as described herein.

5.2.6. Exemplary Data Analysis

In some embodiments, the presence of Flu, RSV, and/or SARS-CoV-2 is detected if the Ct value for any one of the Flu, RSV, or SARS-CoV-2 target genes (such as PA, PB2, MP, NS, RSV A, RSV B, SARS-CoV-2 E, and SARS-CoV-2 N2) is below a certain threshold. In some embodiments, the valid range of Ct values is less than 40 cycles. In some embodiments the valid range of Ct values is 12 to 39.9 Ct. In some such embodiments, if no amplification above background is observed from the Flu-specific primers after 40 cycles, the sample is considered to be negative for Flu. In some such embodiments, the sample is considered to be negative for Flu only if amplification of the exogenous control (SPC) is above background.

In some embodiments, a computer-based analysis program is used to translate the raw data generated by the detection assay into data of predictive value for a clinician. The clinician can access the predictive data using any suitable means. Thus, in some embodiments, the present invention provides the further benefit that the clinician, who is not likely to be trained in genetics or molecular biology, need not understand the raw data. The data is presented directly to the clinician in its most useful form. The clinician is then able to immediately utilize the information in order to optimize the care of the subject.

The present invention contemplates any method capable of receiving, processing, and transmitting the information to and from laboratories conducting the assays, information provides, medical personal, and subjects. For example, in some embodiments of the present invention, a sample (e.g., a biopsy or a serum or urine sample) is obtained from a subject and submitted to a profiling service (e.g., clinical lab at a medical facility, genomic profiling business, etc.), located in any part of the world (e.g., in a country different than the country where the subject resides or where the information is ultimately used) to generate raw data. Where the sample comprises a tissue or other biological sample, the subject may visit a medical center to have the sample obtained and sent to the profiling center, or subjects may collect the sample themselves (e.g., a urine sample or sputum sample) and directly send it to a profiling center. Where the sample comprises previously determined biological information, the information may be directly sent to the profiling service by the subject (e.g., an information card containing the information may be scanned by a computer and the data transmitted to a computer of the profiling center using an electronic communication systems). Once received by the profiling service, the sample is processed and a profile is produced (i.e., expression data), specific for the diagnostic or prognostic information desired for the subject.

The profile data is then prepared in a format suitable for interpretation by a treating clinician. For example, rather than providing raw expression data, the prepared format may represent a diagnosis or risk assessment (e.g., presence of Flu) for the subject, with or without recommendations for particular treatment options. The data may be displayed to the clinician by any suitable method. For example, in some embodiments, the profiling service generates a report that can be printed for the clinician (e.g., at the point of care) or displayed to the clinician on a computer monitor.

In some embodiments, the information is first analyzed at the point of care or at a regional facility. The raw data is then sent to a central processing facility for further analysis and/or to convert the raw data to information useful for a clinician or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers.

In some embodiments, the subject is able to directly access the data using the electronic communication system. The subject may chose further intervention or counseling based on the results. In some embodiments, the data is used for research use. For example, the data may be used to further optimize the inclusion or elimination of markers as useful indicators of a particular condition or stage of disease or as a companion diagnostic to determine a treatment course of action.

5.2.7. Exemplary Polynucleotides

In some embodiments, polynucleotides are provided. In some embodiments, synthetic polynucleotides are provided. Synthetic polynucleotides, as used herein, refer to polynucleotides that have been synthesized in vitro either chemically or enzymatically. Chemical synthesis of polynucleotides includes, but is not limited to, synthesis using polynucleotide synthesizers, such as OligoPilot™ (GE Healthcare), ABI 3900 DNA Synthesizer (Applied Biosystems), and the like. Enzymatic synthesis includes, but is not limited, to producing polynucleotides by enzymatic amplification, e.g., PCR. A polynucleotide may comprise one or more nucleotide analogs (i.e., modified nucleotides) discussed herein.

In some embodiments, a polynucleotide is provided that comprises a region that is at least 85%, at least 90%, at least 95%, or 100% identical to, or at least 85%, at least 90%, at least 95%, or 100% complementary to, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 contiguous nucleotides of the Flu A polymerase acidic (PA) gene, Flu A polymerase basic 2 (PB2) gene, Flu A matrix protein (MP) gene, Flu B matrix protein (MP) gene, Flu B nonstructural protein (NS) gene, avian influenza MP gene, RSV A genome, RSV B genome, SARS-CoV-2 E gene, and/or SARS-CoV-2 N2 gene, and/or SARS CoV-2 RdRP gene. In some embodiments, a polynucleotide is provided that comprises a region that is at least 85%, at least 90%, at least 95%, or 100% identical to, or complementary to, a span of 6 to 100, 8 to 100, 8 to 75, 8 to 50, 8 to 40, or 8 to 30 contiguous nucleotides of the Flu A polymerase acidic (PA) gene, Flu A polymerase basic 2 (PB2) gene, Flu A matrix protein (MP) gene, Flu B matrix protein (MP) gene, Flu B nonstructural protein (NS) gene, avian MP gene, RSV A genome, RSV B genome, SARS-CoV-2 E gene, and/or SARS-CoV-2 N2 gene, and/or SARS CoV-2 RdRP gene. Nonlimiting exemplary polynucleotides are shown in Table A.

In some embodiments the primers and probes for the N2 gene are specific for SARS-CoV-2 and do not detect other closely related coronaviruses in the Sarbecovirus subgenus such as SARS-CoV-1. This provides a high level of specificity for SARS-CoV-2.

In some embodiments the primers and probes for the E gene are “Sarbecovirus specific” and will also detect other coronaviruses in the Sarbecovirus subgenus in addition to SARS-CoV-2. Since SARS-CoV-2 is the only member of the Sarbecovirus subgenus known to currently circulated in humans, it is in effect specific for SARS-CoV-2. However, in the event another Sarbecovirus emerges in the human population in the future, the primers and probes disclosed herein for the E gene may be used for detection.

In various embodiments, a polynucleotide comprises fewer than 500, fewer than 300, fewer than 200, fewer than 150, fewer than 100, fewer than 75, fewer than 50, fewer than 40, or fewer than 30 nucleotides. In various embodiments, a polynucleotide is between 6 and 200, between 8 and 200, between 8 and 150, between 8 and 100, between 8 and 75, between 8 and 50, between 8 and 40, between 8 and 30, between 15 and 100, between 15 and 75, between 15 and 50, between 15 and 40, or between 15 and 30 nucleotides long.

In some embodiments, the polynucleotide is a primer. In some embodiments, the primer is labeled with a detectable moiety. In some embodiments, a primer is not labeled. A primer, as used herein, is a polynucleotide that is capable of selectively hybridizing to a target RNA or to a cDNA reverse transcribed from the target RNA or to an amplicon that has been amplified from a target RNA or a cDNA (collectively referred to as “template”), and, in the presence of the template, a polymerase and suitable buffers and reagents, can be extended to form a primer extension product.

In some embodiments, the polynucleotide is a probe. In some embodiments, the probe is labeled with a detectable moiety. A detectable moiety, as used herein, includes both directly detectable moieties, such as fluorescent dyes, and indirectly detectable moieties, such as members of binding pairs. When the detectable moiety is a member of a binding pair, in some embodiments, the probe can be detectable by incubating the probe with a detectable label bound to the second member of the binding pair. In some embodiments, a probe is not labeled, such as when a probe is a capture probe, e.g., on a microarray or bead. In some embodiments, a probe is not extendable, e.g., by a polymerase. In other embodiments, a probe is extendable.

In some embodiments, the polynucleotide is a FRET probe that in some embodiments is labeled at the 5′-end with a fluorescent dye (donor) and at the 3′-end with a quencher (acceptor), a chemical group that absorbs (i.e., suppresses) fluorescence emission from the dye when the groups are in close proximity (i.e., attached to the same probe). Thus, in some embodiments, the emission spectrum of the dye should overlap considerably with the absorption spectrum of the quencher. In other embodiments, the dye and quencher are not at the ends of the FRET probe.

5.2.7.1. Exemplary Polynucleotide Modifications

In some embodiments, the methods of detecting at least one target gene described herein employ one or more polynucleotides that have been modified, such as polynucleotides comprising one or more affinity-enhancing nucleotide analogs. Modified polynucleotides useful in the methods described herein include primers for reverse transcription, PCR amplification primers, and probes. In some embodiments, the incorporation of affinity-enhancing nucleotides increases the binding affinity and specificity of a polynucleotide for its target nucleic acid as compared to polynucleotides that contain only deoxyribonucleotides, and allows for the use of shorter polynucleotides or for shorter regions of complementarity between the polynucleotide and the target nucleic acid.

In some embodiments, affinity-enhancing nucleotide analogs include nucleotides comprising one or more base modifications, sugar modifications and/or backbone modifications.

In some embodiments, modified bases for use in affinity-enhancing nucleotide analogs include 5-methylcytosine, isocytosine, pseudoisocytosine, 5-bromouracil, 5-propynyluracil, 6-aminopurine, 2-aminopurine, inosine, diaminopurine, 2-chloro-6-aminopurine, xanthine and hypoxanthine.

In some embodiments, affinity-enhancing nucleotide analogs include nucleotides having modified sugars such as 2′-substituted sugars, such as 2′-O-alkyl-ribose sugars, 2′-amino-deoxyribose sugars, 2′-fluoro-deoxyribose sugars, 2′-fluoro-arabinose sugars, and 2′-O-methoxyethyl-ribose (2′MOE) sugars. In some embodiments, modified sugars are arabinose sugars, or d-arabino-hexitol sugars.

In some embodiments, affinity-enhancing nucleotide analogs include backbone modifications such as the use of peptide nucleic acids (PNA; e.g., an oligomer including nucleobases linked together by an amino acid backbone). Other backbone modifications include phosphorothioate linkages, phosphodiester modified nucleic acids, combinations of phosphodiester and phosphorothioate nucleic acid, methylphosphonate, alkylphosphonates, phosphate esters, alkylphosphonothioates, phosphoramidates, carbamates, carbonates, phosphate triesters, acetamidates, carboxymethyl esters, methylphosphorothioate, phosphorodithioate, p-ethoxy, and combinations thereof.

In some embodiments, a polynucleotide includes at least one affinity-enhancing nucleotide analog that has a modified base, at least nucleotide (which may be the same nucleotide) that has a modified sugar, and/or at least one internucleotide linkage that is non-naturally occurring.

In some embodiments, an affinity-enhancing nucleotide analog contains a locked nucleic acid (“LNA”) sugar, which is a bicyclic sugar. In some embodiments, a polynucleotide for use in the methods described herein comprises one or more nucleotides having an LNA sugar. In some embodiments, a polynucleotide contains one or more regions consisting of nucleotides with LNA sugars. In other embodiments, a polynucleotide contains nucleotides with LNA sugars interspersed with deoxyribonucleotides. See, e.g., Frieden, M. et al. (2008) Curr. Pharm. Des. 14(11):1138-1142.

5.2.7.2. Exemplary Primers

In some embodiments, a primer and primer pairs are provided. In some embodiments, a primer is at least 85%, at least 90%, at least 95%, or 100% identical to, or at least 85%, at least 90%, at least 95%, or 100% complementary to, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 contiguous nucleotides of the Flu A polymerase acidic (PA) gene, Flu A polymerase basic 2 (PB2) gene, Flu A matrix protein (MP) gene, Flu B matrix protein (MP) gene, Flu B nonstructural protein (NS) gene, avian influenza matrix protein (MP) gene, RSV A genome, RSV B genome, SARS-CoV-2 E gene, and/or SARS-CoV-2 N2 gene, and/or SARS CoV-2 RdRP gene.

In some embodiments, a primer is provided that comprises a region of 6 to 100, 8 to 100, 8 to 75, 8 to 50, 8 to 40, or 8 to 30 contiguous nucleotides having a sequence that is at least 85%, at least 90%, at least 95%, or 100% identical to, or complementary to, a span of 6 to 100, 8 to 100, 8 to 75, 8 to 50, 8 to 40, or 8 to 30 contiguous nucleotides of the Flu A polymerase acidic (PA) gene, Flu A polymerase basic 2 (PB2) gene, Flu A matrix protein (MP) gene, Flu B matrix protein (MP) gene, Flu B nonstructural protein (NS) gene, avian influenza matrix protein (MP) gene, RSV A genome, RSV B genome, SARS-CoV-2 E gene, and/or SARS-CoV-2 N2 gene, and/or SARS CoV-2 RdRP gene. Nonlimiting exemplary primers are shown in Table A.

In some embodiments, a primer may also comprise portions or regions that are not identical or complementary to the target gene. In some embodiments, a region of a primer that is at least 85%, at least 90%, at least 95%, or 100% identical or complementary to a target gene is contiguous, such that any region of a primer that is not identical or complementary to the target gene does not disrupt the identical or complementary region.

In some embodiments, a primer comprises a portion that is at least 85%, at least 90%, at least 95%, or 100% identical to a region of a target gene. In some such embodiments, a primer that comprises a region that is at least 85%, at least 90%, at least 95%, or 100% identical to a region of the target gene is capable of selectively hybridizing to a cDNA that has been reverse transcribed from the RNA, or to an amplicon that has been produced by amplification of the target gene. In some embodiments, the primer is complementary to a sufficient portion of the cDNA or amplicon such that it selectively hybridizes to the cDNA or amplicon under the conditions of the particular assay being used.

As used herein, “selectively hybridize” means that a polynucleotide, such as a primer or probe, will hybridize to a particular nucleic acid in a sample with at least 5-fold greater affinity than it will hybridize to another nucleic acid present in the same sample that has a different nucleotide sequence in the hybridizing region. Exemplary hybridization conditions are discussed herein, for example, in the context of a reverse transcription reaction or a PCR amplification reaction. In some embodiments, a polynucleotide will hybridize to a particular nucleic acid in a sample with at least 10-fold greater affinity than it will hybridize to another nucleic acid present in the same sample that has a different nucleotide sequence in the hybridizing region.

In some embodiments, a primer is used to reverse transcribe a target RNA, for example, as discussed herein. In some embodiments, a primer is used to amplify a target RNA or a cDNA reverse transcribed therefrom. Such amplification, in some embodiments, is quantitative PCR, for example, as discussed herein.

In some embodiments, a primer comprises a detectable moiety.

In some embodiments, primer pairs are provided. Such primer pairs are designed to amplify a portion of a target gene, such as the Flu A PA gene, Flu A PB2 gene, Flu A matrix protein (MP) gene, Flu B matrix protein (MP) gene, Flu B nonstructural protein (NS) gene, avian influenza MP gene, RSV A genome, RSV B genome, SARS-CoV-2 E gene, SARS-CoV-2 N2 gene, or SARS CoV-2 RdRP gene, or an endogenous control such as a sample adequacy control (SAC), or an exogenous control such as a sample processing control (SPC). In some embodiments two forward primers or two reverse primers may be used to amplify a target gene, resulting in two different ampicons. In some embodiments, a primer pair is designed to produce an amplicon that is 50 to 1500 nucleotides long, 50 to 1000 nucleotides long, 50 to 750 nucleotides long, 50 to 500 nucleotides long, 50 to 400 nucleotides long, 50 to 300 nucleotides long, 50 to 200 nucleotides long, 50 to 150 nucleotides long, 100 to 300 nucleotides long, 100 to 200 nucleotides long, or 100 to 150 nucleotides long. Nonlimiting exemplary primer pairs are shown in Table A.

TABLE A
Primers and Probes for Detecting Flu, RSV, and SARS-
CoV-2

| | | | | | Amplicon |
|---|---|---|---|---|---|
| | | | | SEQ ID | SEQ ID |
| Target | Oligo | Description | Sequence | NO. | NO. |
| Flu A MP | Fwd | Flu A MP fwd | TIC TAA CCG AGG TCG AAA CG | 23 | 10 |
| | Rev | Flu A MP Rev | ATT GOT CTT GTC TTT AGC CA | 24 | |
| | Probe | Flu A MP FAM_Q38 | TCA GGC CCC CTC AAA GCC GA | 25 | |
| Flu A PB2 | Fwd | Flu A PB2 Fwd | AAA CGG GAC TCT AGC ATA CT | 17 | 8 |
| | Rev | Flu A PB2 Rev | TAA TTG ATG GCC ATC CGA AT | 18 | |
| | Probe | Flu A PB2 FAM_Q38 | AGC CAG ACA GCG ACC AAA AG | 19 | |
| Flu A PA | Fwd | Flu A PA Fwd | ATC TTG GGG GGC TAT ATG AAG | 20 | 9 |
| | | | CAA T | | |
| | Rev | Flu APA Rev | AGG AAG GAG TTG AAC CAA GA | 21 | |
| | Probe | Flu A PA FAM_Q38 | AAT GAT CCC TTG GTT TTG CT | 22 | |
| Flu B MP | Fwd | Flu B MP Fwd | TTG GAG ACA CGA TTG CCT AC | 32 | 13 |
| | Rev | Flu B MP Rev | AGG TCA AAT TCT TTC CCA CC | 33 | |
| | Probe | Flu B MP CF3_Q38 | ATG GAG AAG GCA AAG CAG AA | 34 | |
| Flu B NS | Fwd | Flu B NS Fwd | GAT GGC CAT CGG ATC CTC AA | 35 | 14 |
| | Rev | Flu B NS Rev | GCT CTT GAC CAA ATT GGG AT | 36 | |
| | Probe | Flu B NS CF3_Q38 | AAA GCC AAT TCG AGC AGC TG | 37 | |
| RSV A | Fwd | RSV A Fwd | TAC ACT CAA CAA AGA TCA ACT | 38 | 15 |
| | | | TCT GTC | | |
| | Rev | RSV A Rev | CAT GCC ACA TAA CTT ATT GAT | 39 | |
| | | | GIG T | | |
| | Probe | RSV A CF4_Q38 | CAC CAT CCA ACG GAG CAC AGG | 40 | |
| | | | AGA | | |
| | Fwd | RSV A V2 Fwd | TAC ACT CAA CAA AGA TCA ACT | 67 | |
| | | | TCT ATC | | |
| | Rev | RSV A V2a Rev | CAT GCC ACA TAA CTT ATT AAT | 68 | |
| | | | GTG T | | |
| | Rev | RSV A V2b Rev | CAT GCC ACA TAG CTT ATT GAT | 69 | |
| | | | GTG T | | |
| RSV B | Fwd | RSV B Fwd | CAT TAA ATA AGG ATC AGC TGC | 41 | 16 |
| | | | TGT C | | |
| | Rev | RSV B Rev | GCA TAC CAC ATA GTT TGT TTA | 42 | |
| | | | GGT GTT | | |
| | Probe | RSV B CF4_Q38 | TAA TAT TGA TAC TCC CAA TTA | 43 | |
| | | | TGA TGT GC | | |
| Avian MP | Fwd | Avian MP Fwd | CAA GAC CAA TCC TGT CAC CT | 26 | 11 |
| | Rev | Avian MP Rev | CGT CTA CGC TGC AGT CCT CG | 27 | |
| | Probe | Avian MP CF5_Q38 | ACG CTC ACC GTG CCC AGT GA | 28 | |
| CoV-2 E | Fwd | COV E V2 Fwd | GCT TTC GTG GTA TTC TTG CT | 70 | 79 |
| | Rev | COV E V2 Rev | GCA GTA CGC ACA CAA TCG | 71 | |
| | Probe | COV E V2 P | CAC TAG CCATCC TTA CTG CGC | 72 | |
| CoV-2 N2 | Fwd | COV N2 V2 Fwd | AAA AAA TTA CAA ACATTG GCC | 73 | |
| | | | GCA AA | | |
| | Rev | COV N2 Rev/CEP- | GCG CGA CAT TCC GAA GAA C | 52 | |
| | | 19NCOV-N2-REV2 | | | |
| | Probe | COV N2 V2 P1 | CAC AAT TTG CCC CCA GCG CTT | 74 | |
| | | | CAG | | |
| | Probe | COV N2 V2 P2 | CAC AAT TTG CCC CUA GCG CTT | 75 | |
| | | | CAG | | |
| RDRP | Fwd | COV2 RDRP V2 | CAC TTG TTC TTG CTC GCA AA | 76 | 80 |
| | | Fwd | | | |
| | Rev1 | COV2 RDRP Rev/ | CAA ATG TTA AAA ACA CTA TTA | 61 | |
| | | RDRP-W-R2 | GCA TAA G | | |
| | Probe | COV2 RDRP V2 P | CAA CGT GTT GTA GCT TGT CAC | 77 | |
| | Rev2 | COV2 RDRP V2 | CTC ATT AGC TAA TCT ATA GAA | 78 | 81 |
| | | Rev2 | ACG G | | |
| CoV-2 E | Fwd | CEP-CO V2-E-FWD1 | TCG GAA GAG ACA GGT ACG TT | 48 | 46 |
| Alt | Rev | CEP-COV2-E-REV1 | CGC AGT AAG GAT GGC TAG T | 49 | |
| | Probe | CEP-COV2-E- | TGC TTT CGT GGT ATT CTT GCT | 50 | |
| | | PRCF1-3 | | | |
| CoV-2 N2 | Fwd | CEP-19NCOV-N2- | TTA CAA ACA TTG GCC GCA AA | 51 | 47 |
| Alt | | FWD2 | | | |
| | Rev | COV N2 Rev /CEP- | GCG CGA CAT TCC GAA GAA C | 52 | |
| | | 19NCOV-N2-REV2 | | | |
| | Probe | CEP-19NCOV-N2- | ACA ATT TGC CCC CAG CGC TTC | 53 | |
| | | PRCF1-3 | AG | | |
| CoV-2 E | Fwd | COV2-E-W-F1 | ACA GGT ACG TTA ATA GTT AAT | 54 | 66 |
| Alt | | | AGC GT | | |
| | Rev | COV2-E-W-R1 | ATA TTG CAG CAG TAC GCA CAC A | 55 | |
| | Probe | COV2-E-W-P2 | ACA CTA GCC ATC CTT ACT GCG | 56 | |
| | | | CTT CG | | |
| CoV-2 N2 | Fwd | COV2-N2-C-F1 | TTA CAA ACA TTG GCC GCA AA | 57 | |
| Alt | Rev | COV2-N2-C-R1 | GCG CGA CAT TCC GAA GAA | 58 | |
| | Probe | COV2-N2-C-P1 | ACA ATT TGC CCC CAG CGC TTC | 59 | |
| | | | AG | | |
| RDRP Alt | Fwd | W-19NCOV-FWD1 | GTG ARA TGG TCA TGT GTG GCG G | 60 | |
| | Rev | COV2 RDRP Rev/ | CAA ATG TTA AAA ACA CTA TTA | 61 | |
| | | RDRP-W-R2 | GCA TAA G | | |
| | Probe | RDRP-W-P2_Q13 | CAG GTG GAA CCT CAT CAG GAG | 62 | |
| | | | ATG C | | |
| ORF1ab | Fwd | CEP-19NCOV- | GAC AAA TGC TGG TGA TTA C | 63 | |
| | | ORF1AB-FWD11 | | | |
| | Rev | CEP-COV-ORF1AB- | CTT TGA GCG TTT CTG CTG CAA A | 64 | |
| | | REV | | | |
| | Probe | CEP-COV-ORF1AB- | CTA ACA CCT GTA CTG AAA GAC | 65 | |
| | | PRCF1-3 | TCA AGC | | |

5.2.7.3. Exemplary Probes

In various embodiments, methods of detecting the presence of influenza, RSV, and/or SARS-CoV-2 comprise hybridizing nucleic acids of a sample with a probe.

In some embodiments, the probe comprises a portion that is complementary to a target gene, such as the Flu A polymerase acidic (PA) gene, Flu A polymerase basic 2 (PB2) gene, Flu A matrix protein (MP) gene, Flu B matrix protein (MP) gene, Flu B nonstructural protein (NS) gene, avian influenza matrix protein (MP) gene, RSV A genome, RSV B genome, SARS-CoV-2 E gene, SARS-CoV-2 N2 gene, and/or SARS CoV-2 RdRP gene, or an endogenous control such as a sample adequacy control (SAC), or an exogenous control such as a sample processing control (SPC). In some embodiments, the probe comprises a portion that is at least 85%, at least 90%, at least 95%, or 100% identical to a region of the target gene.

In some such embodiments, a probe that is at least 85%, at least 90%, at least 95%, or 100% complementary to a target gene is complementary to a sufficient portion of the target gene such that it selectively hybridizes to the target gene under the conditions of the particular assay being used. In some embodiments, a probe that is complementary to a target gene comprises a region of at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 contiguous nucleotides having a sequence that is at least 85%, at least 90%, at least 95%, or 100% complementary to at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 contiguous nucleotides of the target gene. Nonlimiting exemplary probes are shown in Table A.

A probe that is at least 85%, at least 90%, at least 95%, or 100% complementary to a target gene may also comprise portions or regions that are not complementary to the target gene. In some embodiments, a region of a probe that is at least 85%, at least 90%, at least 95%, or 100% complementary to a target gene is contiguous, such that any region of a probe that is not complementary to the target gene does not disrupt the complementary region.

In some embodiments, the probe comprises a portion that is at least 85%, at least 90%, at least 95%, or 100% identical to a region of the target gene, or an endogenous control such as a sample adequacy control (SAC), or an exogenous control such as a sample processing control (SPC). In some such embodiments, a probe that comprises a region that is at least 85%, at least 90%, at least 95%, or 100% identical to a region of the target gene is capable of selectively hybridizing to a cDNA that has been reverse-transcribed from a target gene or to an amplicon that has been produced by amplification of the target gene. In some embodiments, the probe is at least 85%, at least 90%, at least 95%, or 100% complementary to a sufficient portion of the cDNA or amplicon such that it selectively hybridizes to the cDNA or amplicon under the conditions of the particular assay being used. In some embodiments, a probe that is complementary to a cDNA or amplicon comprises a region that is at least 85%, at least 90%, at least 95%, or 100% complementary to at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 contiguous nucleotides of the cDNA or amplicon. A probe that is at least 85%, at least 90%, at least 95%, or 100% complementary to a cDNA or amplicon may also comprise portions or regions that are not complementary to the cDNA or amplicon. In some embodiments, a region of a probe that is at least 85%, at least 90%, at least 95%, or 100% complementary to a cDNA or amplicon is contiguous, such that any region of a probe that is not complementary to the cDNA or amplicon does not disrupt the complementary region.

In some embodiments, the method of detecting one or more target genes comprises: (a) reverse transcribing a target RNA to produce a cDNA that is complementary to the target RNA; (b) amplifying the cDNA from (a); and (c) detecting the amount of a target RNA using real time RT-PCR and a detection probe (which may be simultaneous with the amplification step (b)).

As described above, in some embodiments, real time RT-PCR detection may be performed using a FRET probe, which includes, but is not limited to, a TAQMAN® probe, a Molecular beacon probe and a Scorpion probe. In some embodiments, the real time RT-PCR detection is performed with a TAQMAN® probe, i.e., a linear probe that typically has a fluorescent dye covalently bound at one end of the DNA and a quencher molecule covalently bound elsewhere, such as at the other end of, the DNA. The FRET probe comprises a sequence that is complementary to a region of the cDNA or amplicon such that, when the FRET probe is hybridized to the cDNA or amplicon, the dye fluorescence is quenched, and when the probe is digested during amplification of the cDNA or amplicon, the dye is released from the probe and produces a fluorescence signal. In some embodiments, the amount of target gene in the sample is proportional to the amount of fluorescence measured during amplification.

The TAQMAN® probe typically comprises a region of contiguous nucleotides having a sequence that is at least 85%, at least 90%, at least 95%, or 100% identical or complementary to a region of a target gene or its complementary cDNA that is reverse transcribed from the target RNA template (i.e., the sequence of the probe region is complementary to or identically present in the target RNA to be detected) such that the probe is selectively hybridizable to a PCR amplicon of a region of the target gene. In some embodiments, the probe comprises a region of at least 6 contiguous nucleotides having a sequence that is fully complementary to or identically present in a region of a cDNA that has been reverse transcribed from a target gene. In some embodiments, the probe comprises a region that is at least 85%, at least 90%, at least 95%, or 100% identical or complementary to at least 8 contiguous nucleotides, at least 10 contiguous nucleotides, at least 12 contiguous nucleotides, at least 14 contiguous nucleotides, or at least 16 contiguous nucleotides having a sequence that is complementary to or identically present in a region of a cDNA reverse transcribed from a target gene to be detected.

In some embodiments, the region of the amplicon that has a sequence that is at least 85%, at least 90%, at least 95%, or 100% complementary to the TAQMAN® probe sequence is at or near the center of the amplicon molecule. In some embodiments, there are independently at least 2 nucleotides, such as at least 3 nucleotides, such as at least 4 nucleotides, such as at least 5 nucleotides of the amplicon at the 5′-end and at the 3′-end of the region of complementarity.

In some embodiments, Molecular Beacons can be used to detect PCR products. Like TAQMAN® probes, Molecular Beacons use FRET to detect a PCR product via a probe having a fluorescent dye and a quencher attached at the ends of the probe. Unlike TAQMAN® probes, Molecular Beacons remain intact during the PCR cycles. Molecular Beacon probes form a stem-loop structure when free in solution, thereby allowing the dye and quencher to be in close enough proximity to cause fluorescence quenching. When the Molecular Beacon hybridizes to a target, the stem-loop structure is abolished so that the dye and the quencher become separated in space and the dye fluoresces. Molecular Beacons are available, e.g., from Gene Link™ (see genelink.com).

In some embodiments, Scorpion probes can be used as both sequence-specific primers and for PCR product detection. Like Molecular Beacons, Scorpion probes form a stem-loop structure when not hybridized to a target nucleic acid. However, unlike Molecular Beacons, a Scorpion probe achieves both sequence-specific priming and PCR product detection. A fluorescent dye molecule is attached to the 5′-end of the Scorpion probe, and a quencher is attached elsewhere, such as to the 3′-end. The 3′ portion of the probe is complementary to the extension product of the PCR primer, and this complementary portion is linked to the 5′-end of the probe by a non-amplifiable moiety. After the Scorpion primer is extended, the target-specific sequence of the probe binds to its complement within the extended amplicon, thus opening up the stem-loop structure and allowing the dye on the 5′-end to fluoresce and generate a signal. Scorpion probes are available from, e.g., Premier Biosoft International (see premierbiosoft.com).

In some embodiments, labels that can be used on the FRET probes include colorimetric and fluorescent dyes such as Alexa Fluor dyes, BODIPY dyes, such as BODIPY FL; Cascade Blue; Cascade Yellow; coumarin and its derivatives, such as 7-amino-4-methylcoumarin, aminocoumarin and hydroxycoumarin; cyanine dyes, such as Cy3 and Cy5; eosins and erythrosins; fluorescein and its derivatives, such as fluorescein isothiocyanate; macrocyclic chelates of lanthanide ions, such as Quantum Dye™; Marina Blue; Oregon Green; rhodamine dyes, such as rhodamine red, tetramethylrhodamine and rhodamine 6G; Texas Red; fluorescent energy transfer dyes, such as thiazole orange-ethidium heterodimer; and, TOTAB.

Specific examples of dyes include, but are not limited to, those identified above and the following: Alexa Fluor 350, Alexa Fluor 405, Alexa Fluor 430, Alexa Fluor 488, Alexa Fluor 500. Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 647, Alexa Fluor 660, Alexa Fluor 680, Alexa Fluor 700, and, Alexa Fluor 750; amine-reactive BODIPY dyes, such as BODIPY 493/503, BODIPY 530/550, BODIPY 558/568, BODIPY 564/570, BODIPY 576/589, BODIPY 581/591, BODIPY 630/650, BODIPY 650/655, BODIPY FL, BODIPY R6G, BODIPY TMR, and, BODIPY-TR; Cy3, Cy5, 6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, SYPRO, TAMRA, 2′,4′,5′,7′-Tetrabromosulfonefluorescein, and TET.

Examples of dye/quencher pairs (i.e., donor/acceptor pairs) include, but are not limited to, fluorescein/tetramethylrhodamine; IAEDANS/fluorescein; EDANS/dabcyl; fluorescein/fluorescein; BODIPY FL/BODIPY FL; fluorescein/QSY 7 or QSY 9 dyes. When the donor and acceptor are the same, FRET may be detected, in some embodiments, by fluorescence depolarization. Certain specific examples of dye/quencher pairs (i.e., donor/acceptor pairs) include, but are not limited to, Alexa Fluor 350/Alexa Fluor488; Alexa Fluor 488/Alexa Fluor 546; Alexa Fluor 488/Alexa Fluor 555; Alexa Fluor 488/Alexa Fluor 568; Alexa Fluor 488/Alexa Fluor 594; Alexa Fluor 488/Alexa Fluor 647; Alexa Fluor 546/Alexa Fluor 568; Alexa Fluor 546/Alexa Fluor 594; Alexa Fluor 546/Alexa Fluor 647; Alexa Fluor 555/Alexa Fluor 594; Alexa Fluor 555/Alexa Fluor 647; Alexa Fluor 568/Alexa Fluor 647; Alexa Fluor 594/Alexa Fluor 647; Alexa Fluor 350/QSY35; Alexa Fluor 350/dabcyl; Alexa Fluor 488/QSY 35; Alexa Fluor 488/dabcyl; Alexa Fluor 488/QSY 7 or QSY 9; Alexa Fluor 555/QSY 7 or QSY9; Alexa Fluor 568/QSY 7 or QSY 9; Alexa Fluor 568/QSY 21; Alexa Fluor 594/QSY 21; and Alexa Fluor 647/QSY 21. In some instances, the same quencher may be used for multiple dyes, for example, a broad spectrum quencher, such as an Iowa Black® quencher (Integrated DNA Technologies, Coralville, Iowa) or a Black Hole Quencher™ (BHQ™; Sigma-Aldrich, St. Louis, Mo.).

In some embodiments, for example, in a multiplex reaction in which two or more moieties (such as amplicons) are detected simultaneously, each probe comprises a detectably different dye such that the dyes may be distinguished when detected simultaneously in the same reaction. One skilled in the art can select a set of detectably different dyes for use in a multiplex reaction.

In some embodiments where two or more amplicons are detected simultaneously, two or more probes may contain the same detectable dye such that a single dye can be used to detect multiple amplicons simultaneously in the same reaction.

Specific examples of fluorescently labeled ribonucleotides useful in the preparation of PCR probes for use in some embodiments of the methods described herein are available from Molecular Probes (Invitrogen), and these include, Alexa Fluor 488-5-UTP, Fluorescein-12-UTP, BODIPY FL-14-UTP, BODIPY TMR-14-UTP, Tetramethylrhodamine-6-UTP, Alexa Fluor 546-14-UTP, Texas Red-5-UTP, and BODIPY TR-14-UTP. Other fluorescent ribonucleotides are available from Amersham Biosciences (GE Healthcare), such as Cy3-UTP and Cy5-UTP.

Examples of fluorescently labeled deoxyribonucleotides useful in the preparation of PCR probes for use in the methods described herein include Dinitrophenyl (DNP)-1′-dUTP, Cascade Blue-7-dUTP, Alexa Fluor 488-5-dUTP, Fluorescein-12-dUTP, Oregon Green 488-5-dUTP, BODIPY FL-14-dUTP, Rhodamine Green-5-dUTP, Alexa Fluor 532-5-dUTP, BODIPY TMR-14-dUTP, Tetramethylrhodamine-6-dUTP, Alexa Fluor 546-14-dUTP, Alexa Fluor 568-5-dUTP, Texas Red-12-dUTP, Texas Red-5-dUTP, BODIPY TR-14-dUTP, Alexa Fluor 594-5-dUTP, BODIPY 630/650-14-dUTP, BODIPY 650/665-14-dUTP; Alexa Fluor 488-7-OBEA-dCTP, Alexa Fluor 546-16-OBEA-dCTP, Alexa Fluor 594-7-OBEA-dCTP, Alexa Fluor 647-12-OBEA-dCTP. Fluorescently labeled nucleotides are commercially available and can be purchased from, e.g., Invitrogen.

In some embodiments, dyes and other moieties, such as quenchers, are introduced into polynucleotide used in the methods described herein, such as FRET probes, via modified nucleotides. A “modified nucleotide” refers to a nucleotide that has been chemically modified, but still functions as a nucleotide. In some embodiments, the modified nucleotide has a chemical moiety, such as a dye or quencher, covalently attached, and can be introduced into a polynucleotide, for example, by way of solid phase synthesis of the polynucleotide. In other embodiments, the modified nucleotide includes one or more reactive groups that can react with a dye or quencher before, during, or after incorporation of the modified nucleotide into the nucleic acid. In specific embodiments, the modified nucleotide is an amine-modified nucleotide, i.e., a nucleotide that has been modified to have a reactive amine group. In some embodiments, the modified nucleotide comprises a modified base moiety, such as uridine, adenosine, guanosine, and/or cytosine. In specific embodiments, the amine-modified nucleotide is selected from 5-(3-aminoallyl)-UTP; 8-[(4-amino)butyl]-amino-ATP and 8-[(6-amino)butyl]-amino-ATP; N6-(4-amino)butyl-ATP, N6-(6-amino)butyl-ATP, N4-[2,2-oxy-bis-(ethylamine)]-CTP; N6-(6-Amino)hexyl-ATP; 8-[(6-Amino)hexyl]-amino-ATP; 5-propargylamino-CTP, 5-propargylamino-UTP. In some embodiments, nucleotides with different nucleobase moieties are similarly modified, for example, 5-(3-aminoallyl)-GTP instead of 5-(3-aminoallyl)-UTP. Many amine modified nucleotides are commercially available from, e.g., Applied Biosystems, Sigma, Jena Bioscience and TriLink.

Exemplary detectable moieties also include, but are not limited to, members of binding pairs. In some such embodiments, a first member of a binding pair is linked to a polynucleotide. The second member of the binding pair is linked to a detectable label, such as a fluorescent label. When the polynucleotide linked to the first member of the binding pair is incubated with the second member of the binding pair linked to the detectable label, the first and second members of the binding pair associate and the polynucleotide can be detected. Exemplary binding pairs include, but are not limited to, biotin and streptavidin, antibodies and antigens, etc.

In some embodiments, multiple target genes are detected in a single multiplex reaction. In some such embodiments, each probe that is targeted to a unique amplicon is spectrally distinguishable when released from the probe, in which case each target gene is detected by a unique fluorescence signal. In some embodiments, two or more target genes are detected using the same fluorescent signal, in which case detection of that signal indicates the presence of either of the target genes or both.

One skilled in the art can select a suitable detection method for a selected assay, e.g., a real-time RT-PCR assay. The selected detection method need not be a method described above, and may be any method.

5.3. Exemplary Compositions and Kits

In another aspect, compositions are provided. In some embodiments, compositions are provided for use in the methods described herein.

In some embodiments, compositions are provided that comprise at least one target gene-specific primer. The terms “target gene-specific primer” and “target RNA-specific primer” are used interchangeably and encompass primers that have a region of contiguous nucleotides having a sequence that is (i) at least 85%, at least 90%, at least 95%, or 100% identical to a region of a target gene, or (ii) at least 85%, at least 90%, at least 95%, or 100% complementary to the sequence of a region of contiguous nucleotides found in a target gene. In some embodiments, a composition is provided that comprises at least one pair of target gene-specific primers. The term “pair of target gene-specific primers” encompasses pairs of primers that are suitable for amplifying a defined region of a target gene. A pair of target gene-specific primers typically comprises a first primer that comprises a sequence that is at least 85%, at least 90%, at least 95%, or 100% identical to the sequence of a region of a target gene and a second primer that comprises a sequence that is at least 85%, at least 90%, at least 95%, or 100% complementary to a region of a target gene. A pair of primers is typically suitable for amplifying a region of a target gene that is 50 to 1500 nucleotides long, 50 to 1000 nucleotides long, 50 to 750 nucleotides long, 50 to 500 nucleotides long, 50 to 400 nucleotides long, 50 to 300 nucleotides long, 50 to 200 nucleotides long, 50 tO 150 nucleotides long, 100 to 300 nucleotides long, 100 to 200 nucleotides long, or 100 to 150 nucleotides long. Nonlimiting exemplary primers, and pairs of primers, are shown in Table A.

In some embodiments, a composition comprises at least one pair of target gene-specific primers. In some embodiments, a composition additionally comprises a pair of target gene-specific primers for amplifying an endogenous control (such as an SAC) and/or one pair of target gene-specific primers for amplifying an exogenous control (such as an SPC).

In some embodiments, a composition comprises at least one target gene-specific probe. The terms “target gene-specific probe” and “target RNA-specific probe” are used interchangeably and encompass probes that have a region of contiguous nucleotides having a sequence that is (i) at least 85%, at least 90%, at least 95%, or 100% identical to a region of a target gene, or (ii) at least 85%, at least 90%, at least 95%, or 100% complementary to the sequence of a region of contiguous nucleotides found in a target gene. Nonlimiting exemplary target-specific probes are shown in Tables A and B.

In some embodiments, a composition (including a composition described above that comprises one or more pairs of target gene-specific primers) comprises one or more probes for detecting the target genes. In some embodiments, a composition comprises a probe for detecting an endogenous control (such as an SAC) and/or a probe for detecting an exogenous control (such as an SPC).

In some embodiments, a composition is an aqueous composition. In some embodiments, the aqueous composition comprises a buffering component, such as phosphate, tris, HEPES, etc., and/or additional components, as discussed below. In some embodiments, a composition is dry, for example, lyophilized, and suitable for reconstitution by addition of fluid. A dry composition may include one or more buffering components and/or additional components.

In some embodiments, a composition further comprises one or more additional components. Additional components include, but are not limited to, salts, such as NaCl, KCl, and MgCl₂; polymerases, including thermostable polymerases such as Taq; dNTPs; reverse transcriptases, such as MMLV reverse transcriptase; Rnase inhibitors; bovine serum albumin (BSA) and the like; reducing agents, such as β-mercaptoethanol; EDTA and the like; etc. One skilled in the art can select suitable composition components depending on the intended use of the composition.

In some embodiments, compositions are provided that comprise at least one polynucleotide for detecting at least one target gene. In some embodiments, the polynucleotide is used as a primer for a reverse transcriptase reaction. In some embodiments, the polynucleotide is used as a primer for amplification. In some embodiments, the polynucleotide is used as a primer for PCR. In some embodiments, the polynucleotide is used as a probe for detecting at least one target gene. In some embodiments, the polynucleotide is detectably labeled. In some embodiments, the polynucleotide is a FRET probe. In some embodiments, the polynucleotide is a TAQMAN® probe, a Molecular Beacon, or a Scorpion probe.

In some embodiments, a composition comprises at least one FRET probe having a sequence that is at least 85%, at least 90%, at least 95%, or 100% identical, or at least 85%, at least 90%, at least 95%, or 100% complementary, to a region of, a target gene, such as the Flu A polymerase acidic (PA) gene, Flu A polymerase basic 2 (PB2) gene, Flu A matrix protein (MP) gene, Flu B matrix protein (MP) gene, Flu B nonstructural protein (NS) gene, avian influenza matrix protein (MP) gene, RSV A genome, RSV B genome, SARS-CoV-2 E gene, and/or SARS-CoV-2 N2 gene, and/or SARS CoV-2 RdRP gene. In some embodiments, a FRET probe is labeled with a donor/acceptor pair such that when the probe is digested during the PCR reaction, it produces a unique fluorescence emission that is associated with a specific target gene. In some embodiments, when a composition comprises multiple FRET probes, each probe is labeled with a different donor/acceptor pair such that when the probe is digested during the PCR reaction, each one produces a unique fluorescence emission that is associated with a specific probe sequence and/or target gene. In some embodiments, the sequence of the FRET probe is complementary to a target region of a target gene. In other embodiments, the FRET probe has a sequence that comprises one or more base mismatches when compared to the sequence of the best-aligned target region of a target gene.

In some embodiments, a composition comprises a FRET probe consisting of at least 8, at least 9, at least 10, at least 11, at least 13, at least 14, at least 15, at least 16, at least 17, at least 18, at least 19, at least 20, at least 21, at least 22, at least 23, at least 24, or at least 25 nucleotides, wherein at least a portion of the sequence is at least 85%, at least 90%, at least 95%, or 100% identical, or at least 85%, at least 90%, at least 95%, or 100% complementary, to a region of, a target gene, such as the Flu A polymerase acidic (PA) gene, Flu A polymerase basic 2 (PB2) gene, Flu A matrix protein (MP) gene, Flu B matrix protein (MP) gene, Flu B nonstructural protein (NS) gene, avian influenza matrix protein (MP) gene, RSV A genome, RSV B genome, SARS-CoV-2 E gene, and/or SARS-CoV-2 N2 gene, and/or SARS CoV-2 RdRP gene. In some embodiments, at least 8, at least 9, at least 10, at least 11, at least 13, at least 14, at least 15, at least 16, at least 17, at least 18, at least 19, at least 20, at least 21, at least 22, at least 23, at least 24, or at least 25 nucleotides of the FRET probe are identically present in, or complementary to a region of, a target gene, such as the Flu A polymerase acidic (PA) gene, Flu A polymerase basic 2 (PB2) gene, Flu A matrix protein (MP) gene, Flu B matrix protein (MP) gene, Flu B nonstructural protein (NS) gene, avian influenza matrix protein (MP) gene, RSV A genome, RSV B genome, SARS-CoV-2 E gene, and/or SARS-CoV-2 N2 gene, and/or SARS CoV-2 RdRP gene. In some embodiments, the FRET probe has a sequence with one, two or three base mismatches when compared to the sequence or complement of the target gene.

In some embodiments, a kit comprises a polynucleotide discussed above. In some embodiments, a kit comprises at least one primer and/or probe discussed above. In some embodiments, a kit comprises at least one polymerase, such as a thermostable polymerase. In some embodiments, a kit comprises dNTPs. In some embodiments, kits for use in the real time RT-PCR methods described herein comprise one or more target gene-specific FRET probes and/or one or more primers for reverse transcription of target RNAs and/or one or more primers for amplification of target genes or cDNAs reverse transcribed therefrom.

In some embodiments, one or more of the primers and/or probes is “linear.” A “linear” primer refers to a polynucleotide that is a single stranded molecule, and typically does not comprise a short region of, for example, at least 3, 4 or 5 contiguous nucleotides, which are complementary to another region within the same polynucleotide such that the primer forms an internal duplex. In some embodiments, the primers for use in reverse transcription comprise a region of at least 4, such as at least 5, such as at least 6, such as at least 7 or more contiguous nucleotides at the 3′-end that has a sequence that is complementary to region of at least 4, such as at least 5, such as at least 6, such as at least 7 or more contiguous nucleotides at the 5′-end of a target gene.

In some embodiments, a kit comprises one or more pairs of linear primers (a “forward primer” and a “reverse primer”) for amplification of a target gene or cDNA reverse transcribed therefrom. Accordingly, in some embodiments, a first primer comprises a region of at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25 contiguous nucleotides having a sequence that is at least 85%, at least 90%, at least 95%, or 100% identical to the sequence of a region of at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25 contiguous nucleotides at a first location in the target gene. Furthermore, in some embodiments, a second primer comprises a region of at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25 contiguous nucleotides having a sequence that is at least 85%, at least 90%, at least 95%, or 100% complementary to the sequence of a region of at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25 contiguous nucleotides at a second location in the target gene, such that a PCR reaction using the two primers results in an amplicon extending from the first location of the target gene to the second location of the target gene.

In some embodiments, the kit comprises at least two, at least three, or at least four sets of primers, each of which is for amplification of a different target gene or cDNA reverse transcribed therefrom. In some embodiments, the kit further comprises at least one set of primers for amplifying a control RNA, such as an endogenous control and/or an exogenous control.

In some embodiments, probes and/or primers for use in the compositions described herein comprise deoxyribonucleotides. In some embodiments, probes and/or primers for use in the compositions described herein comprise deoxyribonucleotides and one or more nucleotide analogs, such as LNA analogs or other duplex-stabilizing nucleotide analogs described above. In some embodiments, probes and/or primers for use in the compositions described herein comprise all nucleotide analogs. In some embodiments, the probes and/or primers comprise one or more duplex-stabilizing nucleotide analogs, such as LNA analogs, in the region of complementarity.

In some embodiments, the kits for use in real time RT-PCR methods described herein further comprise reagents for use in the reverse transcription and amplification reactions. In some embodiments, the kits comprise enzymes, such as a reverse transcriptase or a heat stable DNA polymerase, such as Taq polymerase. In some embodiments, the kits further comprise deoxyribonucleotide triphosphates (dNTP) for use in reverse transcription and/or in amplification. In further embodiments, the kits comprise buffers optimized for specific hybridization of the probes and primers.

A kit generally includes a package with one or more containers holding the reagents, as one or more separate compositions or, optionally, as an admixture where the compatibility of the reagents will allow. The kit can also include other material(s) that may be desirable from a user standpoint, such as a buffer(s), a diluent(s), a standard(s), and/or any other material useful in sample processing, washing, or conducting any other step of the assay.

Kits preferably include instructions for carrying out one or more of the methods described herein. Instructions included in kits can be affixed to packaging material or can be included as a package insert. While the instructions are typically written or printed materials they are not limited to such. Any medium capable of storing such instructions and communicating them to an end user is contemplated by this invention. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

In some embodiments, the kit can comprise the reagents described above provided in one or more GENEXPERT® cartridge(s). These cartridges permit extraction, amplification, and detection to be carried out within this self-contained “laboratory in a cartridge.” (See, e.g., U.S. Pat. Nos. 5,958,349, 6,403,037, 6,440,725, 6,783,736, and 6,818,185; each of which is herein incorporated by reference in its entirety.) Reagents for measuring genomic copy number level and detecting a pathogen could be provided in separate cartridges within a kit or these reagents (adapted for multiplex detection) could be provide in a single cartridge.

Any of the kits described here can include, in some embodiments, a receptacle for a nasal aspirate/wash sample and/or a swab for collecting a nasopharyngeal swab sample.

The following examples are for illustration purposes only, and are not meant to be limiting in any way.

### 6. EXAMPLES

### 6.1. Example 1

### Design of SARS-CoV-2/Flu/RSV Assay

Suitable gene fragments for the design of primers and probes for detecting influenza and RSV were identified by first generating sequence alignments of RNA segments using the European Molecular Biology Laboratory (EMBL)-European Bioinformatics Institute (EBI) sequence alignment software, ClustalW. ClustalW is a general purpose multiple sequence alignment program for nucleic acids or proteins that calculates the best match for the selected sequences and aligns them such that the identities, similarities, and differences can be compared. For each potential target, sequence regions, 100-200 nt in length, were chosen that differentiated the targets. The regions were also selected based on the frequency of polymorphic base substitutions; regions were selected that were highly conserved. Redundant multi-target design was used to mitigate false negative results.

Design of primers and probes for amplification of RNA fragments in the selected regions was performed using DNA Software, Inc.'s Visual OMP (Oligonucleotide Modeling Platform). Visual OMP models, in silico, the folding and hybridization of single-stranded nucleic acids by incorporating all public domain thermodynamic parameters as well as proprietary nearest-neighbor and multi-state thermodynamic parameters for DNA, RNA, PNA, and Inosine. This enables the effective design of primers and probes for complex assays such as microarrays, microfluidics applications and multiplex PCR. In silico experiments simulate secondary structures for targets (optimal and suboptimal), primers (optimal and suboptimal), homodimers, and target and primer heterodimers, given specified conditions. Values for melting temperature (Tm), free energy (ΔG), percent bound, and concentrations for all species are calculated. Additionally, Visual OMP predicts the binding efficiency between primers and probes with target(s) in a single or multiplex reaction.

Using this software tool, predicted interactions between oligonucleotides and the different Flu targets were evaluated thermodynamically and unwanted interactions were minimized.

The selected primers and probes were then subjected to BLAST searching. Oligos were queried singly and in combinations representing the expected full-length amplicon sequences.

Primers and probes were designed as described above to detect the Flu A PB2 and PA target genes, Flu A 1 matrix protein (MP) gene, Flu A 2 (avian isolates) MP gene, Flu B MP gene, Flu B NS gene, and respiratory syncytial virus (RSV) A and B, as shown in Table A.

Primers and probes for detecting SARS-CoV-2 were designed and added to the panel. Approximately 80 primer/probe combinations screened in simplex, duplex and in the presence of Flu/RSV primer/probes as shown in Table A. Examplary SARS-CoV-2 primers and probes are shown in Table A.

The SARS-CoV-2 Orflab gene was evaluated as a target for detecting SARS-CoV-2. The RDRP gene was also evaluated as a target for detecting SARS-CoV-2.

An examplary primer and probe composition of the SARS-CoV-2/Flu/RSV multiplex assay is shown in Table B.

TABLE B
Primer and probe concentrations

| | Bead Content | Bead Content | Bead Content |
|---|---|---|---|
| | (nmol/bead) | (nmol/bead) | (nmol/bead) |
| Target | Fwd. Primer | Rev. Primer | Probe |
| Flu A MP | 800 | 800 | 150 |
| Flu A PB2 | 800 | 400 | 150 |
| Flu A PA | 400 | 800 | 150 |
| Flu B MP | 200 | 800 | 125 |
| Flu B NS | 800 | 400 | 125 |
| RSV A | 800 | 800 | 400 |
| RSV B | 800 | 800 | 400 |
| Avian MP | 800 | 800 | 400 |
| Control | 300 | 300 | 600 |
| CoV-2 E | 600 | 800 | 300-400 |
| CoV-2 N2 | 800 | 800 | 300-400 |

As set forth in Table B, each of the SARS-CoV-2 E probe and SARS-Cov-2 N2 probe have been shown to optimize the signal to noise ratio in the PCR reaction at a concentration of 300-400nM. The concentration for the SARS-CoV-2 E probe and SARS-CoV-2 N2 probe can also be around 600 nM/bead for each detection probe.

### 6.2. Example 2

### Performance of SARS-CoV-2/Flu/RSV Assay

Table C shows the performance of the SARS-CoV-2/Flu/RSV multiplex assay as compared to an assay detecting just SARS-CoV-2 markers and an assay detecting just Flu/RSV markers. The data in Table C show mean Ct values of 10 tests using quantified reference material at lx LOD for each marker in the 4-plex assay. Controls were run in parallel on the XPERT® Xpress SARS-CoV-2 assay and the XPERT® Xpress Flu RSV assay.

TABLE C
Performance of SARS-CoV-2/Flu/RSV Assay

| | | CoV2 | Flu A1 | Flu A2 | RSV A | RSV B | Flu B |
|---|---|---|---|---|---|---|---|
| | | Ct | Ct | Ct | Ct | Ct | Ct |
| Assay | SARS- | 39.3/39.7 | | | | | |
| | CoV2 | | | | | | |
| | Xpress | | 36.5 | 36.8 | 35.3 | 33 | 33.9 |
| | Flu RSV | | | | | | |
| | Resp | 38.5 | 37.4 | 36.1 | 32.8 | 31.1 | 30.8 |
| | 4plex | | | | | | |

### 6.3. Example 3

### Clinical Performance of SARS-CoV-2/Flu/RSV Assay

6.3.1. Clinical Evaluation

The performance of the SARS-CoV-2/Flu/RSV multiplex assay was evaluated using archived clinical nasopharyngeal (NP) swab specimens in viral transport medium. Archived specimens were selected consecutively by date and previously known analyte result. A total of 240 NP swab specimens were tested with the SARS-CoV-2/Flu/RSV multiplex assay side by side with a SARS-CoV-2 test and the FDA-cleared Xpert® Xpress Flu/RSV test in a randomized and blinded fashion.

Positive Percent Agreement (PPA) and Negative Percent Agreement (NPA) were determined by comparing the results of the SARS-CoV-2/Flu/RSV multiplex assay relative to the results of a SARS-CoV-2 test for the SARS-CoV-2 target, and Xpert® Xpress Flu/RSV for the Flu A, Flu B, and RSV targets, respectively.

The SARS-CoV-2/Flu/RSV multiplex assay demonstrated a PPA and NPA of 97.9% and 100.0% for SARS-CoV-2, respectively; 100.0% and 100.0% for Flu A, respectively; 100.0% and 99.0% for Flu B, respectively; 100.0% and 100.0% for RSV, respectively (Table D).

TABLE D
SARS-CoV-2/Flu/RSV Multiplex Assay Performance Results

| | Number of | | | | | PPA | NPA |
|---|---|---|---|---|---|---|---|
| Target | Specimens | TP | FP | TN | FN | (95% CI) | (95% CI) |
| SARS-CoV-2 | 240 | 46 | 0 | 193 | 1 | 97.9% | 100.0% |
| | | | | | | (88.9%-99.6%) | (98.1%-100.0%) |
| Flu A | 240 | 48 | 0 | 192 | 0 | 100.0% | 100.0% |
| | | | | | | (92.6%-100.0%) | (98.0%-100.0%) |
| Flu B | 240 | 46 | 2 | 192 | 0 | 100.0% | 99.0% |
| | | | | | | (92.3%-100.0%) | (96.3%-99.7%) |
| RSV | 240 | 47 | 0 | 193 | 0 | 100.0% | 100.0% |
| | | | | | | (92.4%-100.0%) | (98.1%-100.0%) |

TP: True Positive;
FP: False Positive;
TN: True Negative;
FN: False Negative;
CI: Confidence Interval

6.3.2. Analyical Sensitivity (Limit of Detection)

The analytical sensitivity of the SARS-CoV-2/Flu/RSV multiplex assay was assessed with one lot of reagent and limiting dilutions of the six respiratory viruses (NATtrol SARS-CoV-2, Flu A H1, Flu H3, Flu B, RSV A and RSV B) into pooled negative clinical NP swab matrix following the guidance in Clinical and Laboratory Standards Institute (CLSI) document EP17-A2. The estimated LoD values as determined by Probit regression analysis were verified using two lots of SARS-CoV-2/Flu/RSV multiplex assay reagents. The verified LoD values for the viruses tested are summarized in Table E.

TABLE E
SARS-CoV-2/Flu/RSV Multiplex Assay Limit of Detection

| Virus/Strain | LoD Concentration | |
|---|---|---|
| SARS-CoV-2 (USA-WA1/2020) | 131 | copies/mL |
| Influenza A/California/7/2009 | 0.004 | TCID₅₀/mL |
| Influenza A/Victoria/361/2011 | 0.087 | TCID₅₀/mL |
| Influenza B/Mass/2/2012 | 0.04 | TCID₅₀/mL |
| RSV A/2/Australia/61 | 0.43 | TCID₅₀/mL |
| RSV B/Wash/18537/62 | 0.22 | TCID₅₀/mL |

6.3.3. Analyical Reactivity (Inclusivity)

The inclusivity of SARS-CoV-2/Flu/RSV multiplex assay was evaluated using in silico analysis of the assay amplicons in relation to 48,461 SARS-CoV-2 sequences available in the GISAID gene database for two targets, E and N2. Similar analysis was performed for the RdRP target.

For analysis of the E target, 113 sequences were excluded due to ambiguous nucleotides, which reduced the total to 48,348 sequences. Of the 48,348 GISAID sequences, 48,108 (99.5%) were an exact match to the SARS-CoV-2 E target amplicon generated in the SARS-CoV-2/Flu/RSV multiplex assay. Single nucleotide mismatches were observed for 223 sequences and two mismatches were observed for 17 sequences. Of the 17 sequences with two mismatches, two sequences contained 2 mismatches in the forward primer region, three sequences have a ‘GA′’ dinucleotide in the reverse primer, and twelve sequences contained a ‘AA’ dinucleotide that lies between the oligonucleotides used in the assay. None of these mismatches are expected to affect the performance of the assay.

For analysis of the N2 target, 129 sequences were excluded due to ambiguous nucleotides, which reduced the total used in the evaluation to 48,332 sequences. Of the 48,332 GISAID sequences, 47,962 (99.2%) were an exact match to the SARS-CoV-2 N2 target amplicon generated in the SARS-CoV-2/Flu/RSV multiplex assay. Single nucleotide mismatches were observed for 369 sequences and three (3) mismatches were observed for one sequence. For the one sequence with three variant positions, two of the mismatched nucleotides are in the probe region and could have an impact on probe binding. None of the other mismatches are predicted to have a negative impact on the performance of the assay.

The inclusivity of the SARS-CoV-2/Flu/RSV multiplex assay for Flu and RSV viruses are as reported for the analytical reactivity evaluation of the Xpert® Xpress Flu/RSV test. An in silico analysis of RSV sequences in the public databases revealed some deposited sequences in the NCBI database that might go undetected based on some primer and probe designs, particularly for RSV A. The primers and probes disclosed herein for RSV provide for improved coverage of RSV A.

Xpert® Xpress Flu/RSV test was evaluated against multiple strains of influenza A H1N1 (seasonal pre-2009), influenza A H1N1 (pandemic 2009), influenza A H3N2 (seasonal), avian influenza A (H5N1, H5N2, H6N2, H7N2, H7N3, H2N2, H7N9, and H9N2), influenza B (representing strains from both Victoria and Yamagata lineages), and respiratory syncytial virus subgroups A and B (RSV A and RSV B) at levels near the analytical LoD. A total of 53 strains comprised of 48 influenza viruses (35 influenza A and 13 influenza B) and 5 RSV strains were tested in this study with the Xpert® Xpress Flu/RSV test. Three replicates were tested for each strain. All Flu and RSV strains tested positive in all three replicates, except for one Flu A H1N1 strain (A/New Jersey/8/76), which tested positive in 2 of 3 replicates at 0.1 TCIDso/mL. Results are shown in Table F. Predicted cross reactivity from in silico analyses showed 100% sequence homology for additional pH1N1 strains.

TABLE F
Analytical Reactivity (Inclusivity) of the Xpert ® Xpress Flu/RSV Test

| | | | | Result | | |
|---|---|---|---|---|---|---|
| Virus | Strain | Target Concentration | | Flu A | Flu B | RSV |
| No Template Control | | N/A | | NEG | NEG | NEG |
| Influenza A | A/swine/Iowa/15/30 | 0.1 | TCID₅₀/mL | POS | NEG | NEG |
| H1N1 (pre-2009) | A/WS/33 | 0.1 | TCID₅₀/mL | POS | NEG | NEG |
| | A/PR/8/34 | 0.1 | TCID₅₀/mL | POS | NEG | NEG |
| | A/Mal/302/54 | 0.1 | TCID₅₀/mL | POS | NEG | NEG |
| | A/Denver/1/57 | 0.1 | TCID₅₀/mL | POS | NEG | NEG |
| | A/New Jersey/8/76 | 0.1 | TCID₅₀/mL | POS | NEG | NEG |
| | A/New Caledonia/20/1999 | 0.1 | TCID₅₀/mL | POS | NEG | NEG |
| | A/New York/55/2004 | 0.1 | TCID₅₀/mL | POS | NEG | NEG |
| | A/Solomon Island/3/2006 | 0.1 | TCID₅₀/mL | POS | NEG | NEG |
| | A/Taiwan/42/06 | 0.1 | TCID₅₀/mL | POS | NEG | NEG |
| | A/Brisbane/59/2007 | 0.1 | TCID₅₀/mL | POS | NEG | NEG |
| Influenza A | A/swme/NY/02/2009 | 0.1 | TCID₅₀/mL | POS | NEG | NEG |
| H1N1 (pdm2009) | A/Colorado/14/2012 | 0.1 | TCID₅₀/mL | POS | NEG | NEG |
| | A/Washington/24/2012 | 0.1 | TCID₅₀/mL | POS | NEG | NEG |
| Influenza A | A/Aichi/2/68 | 2.0 | TCID₅₀/mL | POS | NEG | NEG |
| H3N2 (Seasonal) | A/Hong Kong/8/68 | 2.0 | TCID₅₀/mL | POS | NEG | NEG |
| | A/Port Chalmers/1/73 | 2.0 | TCID₅₀/mL | POS | NEG | NEG |
| | A/Hawaii/15/2001 | 2.0 | TCID₅₀/mL | POS | NEG | NEG |
| | A/Wisconsin/67/05 | 2.0 | TCID₅₀/mL | POS | NEG | NEG |
| | A/Brisbane/10/2007 | 2.0 | TCID₅₀/mL | POS | NEG | NEG |
| | A/Minnesota/11/2010 (H3N2)v | 2.0 | TCID₅₀/mL | POS | NEG | NEG |
| | A/Indiana/08/2011 (H3N2)v | 2.0 | TCID₅₀/mL | POS | NEG | NEG |
| | A/Texas/50/2012 | 2.0 | TCID₅₀/mL | POS | NEG | NEG |
| Avian | A/duck/Hunan/795/2002 (H5N1) | ≤1 | ρg/μL^{a} | POS | NEG | NEG |
| influenza A | A/chicken/Hubei/327/2004 (H5N1) | ≤1 | ρg/μL^{a} | POS | NEG | NEG |
| | A/Anhui/01/2005 (H5N1) | ≤1 | ρg/μL^{a} | POS | NEG | NEG |
| | A/Japanese white eye/Hong Kong/ | ≤1 | ρg/μL^{a} | POS | NEG | NEG |
| | 1038/2006 (H5N1) | | | | | |
| | A/mallard/WI/34/75 (H5N2) | ≤1 | ρg/μL^{a} | POS | NEG | NEG |
| | A/chicken/CA431/00 (H6N2) | ≤1 | ρg/μL^{a} | POS | NEG | NEG |
| | A/duck/LTC-10-82743/1943 (H7N2) | ≤1 | ρg/μL^{a} | POS | NEG | NEG |
| | A/chicken/NJ/15086-3/94 (H7N3) | ≤1 | ρg/μL^{a} | POS | NEG | NEG |
| | A/Anhui/1/2013 (H7N9) | N/A^{b} | | POS | NEG | NEG |
| | A/Shanghai/1/2013 (H7N9) | N/A^{b} | | POS | NEG | NEG |
| | A/chicken/Korea/38349-p96323/1996 | ≤1 | ρg/μL^{a} | POS | NEG | NEG |
| | (H9N2) | | | | | |
| | A/Mallard/NY/6750/78 (H2N2) | ≤1 | ρg/μL^{a} | POS | NEG | NEG |
| Influenza B | B/Lee/40 | 1.0 | TCID₅₀/mL | NEG | POS | NEG |
| | B/Allen/45 | 1.0 | TCID₅₀/mL | NEG | POS | NEG |
| | B/GL/1739/54 | 1.0 | TCID₅₀/mL | NEG | POS | NEG |
| | B/Maryland/1/59 | 1.0 | TCID₅₀/mL | NEG | POS | NEG |
| | B/Panama/45/90^{c} | 1.0 | TCID₅₀/mL | NEG | POS | NEG |
| | B/Florida/07/2004^{d} | 1.0 | TCID₅₀/mL | NEG | POS | NEG |
| | B/Florida/02/06^{c} | 1.0 | TCID₅₀/mL | NEG | POS | NEG |
| | B/Florida/04/06^{d} | 1.0 | TCID₅₀/mL | NEG | POS | NEG |
| | B/Hong Kong/5/72 | 1.0 | TCID₅₀/mL | NEG | POS | NEG |
| | B/Wisconsin/01/2011^{d} | 1.0 | TCID₅₀/mL | NEG | POS | NEG |
| | B/Malaysia/2506/04^{c} | 1.0 | TCID₅₀/mL | NEG | POS | NEG |
| | B/Taiwan/2/62 | 1.0 | TCID₅₀/mL | NEG | POS | NEG |
| | B/Brisbane/60/2008^{c} | 1.0 | TCID₅₀/mL | NEG | POS | NEG |
| RSV A | RSV-A/NY (Clinical unknown) | 3.0 | TCID₅₀/mL | NEG | NEG | POS |
| | RSV-A/WI/629-8-2/2007 | 3.0 | TCID₅₀/mL | NEG | NEG | POS |
| | RSV-A/WI/629-11-1/2008 | 3.0 | TCID₅₀/mL | NEG | NEG | POS |
| RSV B | RSV-B/WV14617/85 | 7.0 | TCID₅₀/mL | NEG | NEG | POS |
| | RSV-B/CH93(18)-18 | 7.0 | TCID₅₀/mL | NEG | NEG | POS |

^{a}Purified viral RNA in simulated background matrix was used for avian influenza A viruses due to biosafety regulations.
^{b}Inactivated avian influenza A (H7N9) viruses without viral titer was diluted 100,000-fold in simulated background matrix and tested due to biosafety regulations.
^{c}Known Victoria lineage.
^{d}Known Yamagata lineage.

6.3.4. Analyical Specificity (Exclusivity)

An in silico analysis for possible cross-reactions with all the organisms listed in Table G was conducted by mapping primers and probes in the SARS-CoV-2/Flu/RSV multiplex assay individually to the sequences downloaded from the GISAID database. E primers and probes are not specific for SARS-CoV-2 and will detect Human and Bat SARS-coronavirus. No potential unintended cross reactivity with other organisms listed in Table G is expected based on the in silico analysis.

TABLE G
SARS-CoV-2/Flu/RSV Multiplex Assay Analytical
Specificity Microorganisms

| Microorganisms from the Same | |
|---|---|
| Genetic Family | High Priority Organisms |
| Human coronavirus 229E | Adenovirus (e.g. C1 Ad. 71) |
| Human coronavirus OC43 | Human metapneumovirus (hMPV) |
| Human coronavirus HKU1 | Parainfluenza viruses 1-4 |
| Human coronavirus NL63 | Influenza A |
| SARS-coronavirus | Influenza B |
| MERS-coronavirus | Influenza C |
| Bat coronavirus | Enterovirus (e.g. EV68) |
| | Respiratory syncytial virus |
| | Rhinovirus |
| | Chlamydia pneumoniae |
| | Haemophilus influenzae |
| | Legionella pneumophila |
| | Mycobacterium tuberculosis |
| | Streptococcus pneumoniae |
| | Streptococcus pyogenes |
| | Bordetella pertussis |
| | Mycoplasma pneumoniae |
| | Pneumocystis jirovecii (PJP) |
| | Parechovirus |
| | Candida albicans |
| | Corynebacterium diphtheriae |
| | Legionella non-pneumophila |
| | Bacillus anthracis (Anthrax) |
| | Moraxella catarrhalis |
| | Neisseria elongata and N. meningitidis |
| | Pseudomonas aeruginosa |
| | Staphylococcus epidermidis |
| | Staphylococcus salivarius |
| | Leptospira |
| | Chlamydia psittaci |
| | Coxiella burnetii (Q-Fever) |
| | Staphylococcus aureus |

The analytical specificity of the SARS-CoV-2/Flu/RSV multiplex assay for Flu A, Flu B and RSV viruses are as reported for the analytical exclusivity evaluation of the Xpert® Xpress Flu/RSV test. The analytical specificity of the Xpert® Xpress Flu/RSV test was evaluated by testing a panel of 44 cultures consisting of 16 viral, 26 bacterial, and two yeast strains representing common respiratory pathogens or those potentially encountered in the nasopharynx. Three replicates of each bacterial and yeast strain were tested at concentrations of >1×10⁶ CFU/mL with the exception of one strain that was tested at 1×10⁵ CFU/mL (Chlamydia pneumoniae). Three replicates of each virus were tested at concentrations of ≥1×10⁵ TCID₅₀/mL. The analytical specificity was 100%. Results are shown in Table H.

TABLE H
Analytical Specificity of the Xpert ® Xpress Flu/RSV Test

| Organism | Concentration | | Influenza A | Influenza B | RSV |
|---|---|---|---|---|---|
| No Template Control | N/A | | NEG | NEG | NEG |
| Adenovirus Type 1 | 1.12E+06 | TCID₅₀/mL | NEG | NEG | NEG |
| Adenovirus Type 7 | 1.87E+05 | TCID₅₀/mL | NEG | NEG | NEG |
| Human coronavirus OC43 | 2.85E+05 | TCID₅₀/mL | NEG | NEG | NEG |
| Human coronavirus 229E | 1.00E+05 | TCID₅₀/mL | NEG | NEG | NEG |
| Cytomegalovirus | 1.00E+05 | TCID₅₀/mL | NEG | NEG | NEG |
| Echovirus | 3.31E+07 | TCID₅₀/mL | NEG | NEG | NEG |
| Enterovirus | 3.55E+05 | TCID₅₀/mL | NEG | NEG | NEG |
| Epstein Barr Virus | 7.16E+07 | TCID₅₀/mL | NEG | NEG | NEG |
| Herpes simplex virus | 8.90E+05 | TCID₅₀/mL | NEG | NEG | NEG |
| Measles | 6.31E+05 | TCID₅₀/mL | NEG | NEG | NEG |
| Human metapneumovirus | 1.00E+05 | TCID₅₀/mL | NEG | NEG | NEG |
| Mumps virus | 6.31E+06 | TCID₅₀/mL | NEG | NEG | NEG |
| Human parainfluenza | 1.15E+06 | TCID₅₀/mL | NEG | NEG | NEG |
| virus Type 1 | | | | | |
| Human parainfluenza | 6.31E+05 | TCID₅₀/mL | NEG | NEG | NEG |
| virus Type 2 | | | | | |
| Human parainfluenza | 3.55E+06 | TCID₅₀/mL | NEG | NEG | NEG |
| virus Type 3 | | | | | |
| Rhino virus Type 1A | 1.26E+05 | TCID₅₀/mL | NEG | NEG | NEG |
| Acinetobacter baumannii | 1.00E+06 | CFU/mL | NEG | NEG | NEG |
| Burkholderia cepacia | 3.30E+06 | CFU/mL | NEG | NEG | NEG |
| Candida albicans | 3.20E+06 | CFU/mL | NEG | NEG | NEG |
| Candida parapsilosis | 3.00E+06 | CFU/mL | NEG | NEG | NEG |
| Bordetella pertussis | 3.30E+06 | CFU/mL | NEG | NEG | NEG |
| Chlamydia pneumoniae | 1.00E+05 | CFU/mL | NEG | NEG | NEG |
| Citrobacter freundii | 3.30E+06 | CFU/mL | NEG | NEG | NEG |
| Corynebacterium sp. | 3.30E+06 | CFU/mL | NEG | NEG | NEG |
| Escherichia coli | 1.00E+07 | CFU/mL | NEG | NEG | NEG |
| Enterococcus faecalis | 1.30E+06 | CFU/mL | NEG | NEG | NEG |
| Hemophilus influenzae | 1.00E+06 | CFU/mL | NEG | NEG | NEG |
| Lactobacillus reuteri | 1.00E+06 | CFU/mL | NEG | NEG | NEG |
| Legionella spp. | 1.00E+06 | CFU/mL | NEG | NEG | NEG |
| Moraxella catarrhalis | 1.00E+07 | CFU/mL | NEG | NEG | NEG |
| Mycobacterium | 1.00E+06 | CFU/mL | NEG | NEG | NEG |
| tuberculosis (avirulent) | | | | | |
| Mycoplasma pneumoniae | 1.00E+06 | CFU/mL | NEG | NEG | NEG |
| Neisseria meningitidis | 2.15E+06 | CFU/mL | NEG | NEG | NEG |
| Neisseria mucosa | 1.00E+07 | CFU/mL | NEG | NEG | NEG |
| Propionibacterium acnes | 2.40E+07 | CFU/mL | NEG | NEG | NEG |
| Pseudomonas aeruginosa | 3.70E+06 | CFU/mL | NEG | NEG | NEG |
| Staphylococcus aureus | 2.20E+06 | CFU/mL | NEG | NEG | NEG |
| (protein A producer) | | | | | |
| Staphylococcus | 3.40E+06 | CFU/mL | NEG | NEG | NEG |
| epidermidis | | | | | |
| Staphylcoccus | 4.00E+06 | CFU/mL | NEG | NEG | NEG |
| haemolyticus | | | | | |
| Streptococcus agalactiae | 3.50E+06 | CFU/mL | NEG | NEG | NEG |
| Streptococcus | 1.00E+06 | CFU/mL | NEG | NEG | NEG |
| pneumoniae | | | | | |
| Streptococcus pyogenes | 1.00E+07 | CFU/mL | NEG | NEG | NEG |
| Streptococcus salivarius | 1.00E+07 | CFU/mL | NEG | NEG | NEG |
| Streptococcus sanguinis | 3.10E+06 | CFU/mL | NEG | NEG | NEG |

6.3.5. Competitive Interference

Competitive interference of the SARS-CoV-2/Flu/RSV multiplex assay caused by co-infections were evaluated by testing individual SARS-CoV-2, Flu A, Flu B or RSV strains near the LoD in the presence of different target strains at a higher concentration in a simulated background matrix. The concentration at LoD was 131 copies/mL for SARS-CoV-2 and ranged from 0.04 TCID₅₀/mL to 0.43 TCID₅₀/mL for Flu and RSV strains; the competitive strains was evaluated at 10⁴ titer units (copies/mL, TCID₅₀/mL, CEID₅₀/mL or PFU/mL). Analytical competitive interference was assessed using a strain of SARS-CoV-2 (inactivated USA-WA1/2020), Flu A H3 (H3/Victoria/361/2011), Flu B (B/Mass/02/2012), RSV A (RSV-A/2/Australia/61), and RSV B (RSV-B/Wash/18537/62). Replicates of 20 were tested for each target strain and each competitive strain combination. The normal binomial distribution with 20 replicate samples at LoD is between 17 and 20 positive results based on the binomial distribution with N=20, p=0.95 (X-Bin(20,0.95)). Therefore, sets of 20 with 16 or less positives would be rare and an indication of a competitive inhibitory effect due to high levels of a competing analyte.

With SARS-CoV-2 at 131 copies/mL, competitive inhibitory effects were observed in the presence of Flu A/Victoria/361/2011 at 1×10⁴ CEID₅₀/mL. No competitive inhibitory effects were observed at a lower concentration of Flu A/Victoria/361/2011 (1×10³ CEID₅₀/mL).

With SARS-CoV-2 at 131 copies/mL, competitive inhibitory effects were observed in the presence of Flu B/Mass/2/2012 at 1×10⁴, 1×10³ and 1×10² TCID₅₀/mL. No competitive inhibitory effects were observed at a lower concentration of Flu B/Mass/2/2012 (1×10¹ TCID₅₀₋/mL).

When the SARS-CoV-2 was increased to 1310 copies/mL (10×LoD), competitive inhibitory effects were no longer observed with Flu B/Mass/2/2012 at 1×10⁴ TCID₅₀/mL.

With SARS-CoV-2 at 131 copies/mL in the presence of RSV A/2/Australia/61 at 1×10⁴ PFU/mL or RSV B/Wash/18537/62 at 1×10⁴ TCID₅₀/mL, no competitive inhibitory effects were observed.

With Flu A/Victoria/361/2011 at 0.087 TCID₅₀/mL in the presence of 1×10⁴ copies/mL SARS-CoV-2 virus, no competitive inhibitory effect was observed.

With Flu B/Mass/2/2012 at 0.04 TCID₅₀/mL, competitive inhibitory effects were observed in the presence of Flu A/Victoria/361/2011 at 1×10⁴ CEID₅₀/mL. No competitive inhibitory effects were observed at a lower concentration of Flu A/Victoria/361/2011 (1×10³ CEID₅₀/mL).

With Flu B/Mass/2/2012 at 0.04 TCID₅₀/mL in the presence of 1×10⁴ copies/mL SARS-CoV-2 virus, no competitive inhibitory effects were observed.

With RSV A/2/Australia/61 at 0.43 TCID₅₀/mL, competitive inhibitory effects were observed in the presence of Flu A/Victoria/361/2011 at 1×10⁴ CEID₅₀/mL. No competitive inhibitory effects were observed at a lower concentration of Flu A/Victoria/361/2011 (1×10³ CEID₅₀/mL).

With RSV A/2/Australia/61 at 0.43 TCID₅₀/mL in the presence of 1×10⁴ copies/mL SARS-CoV-2 virus, no competitive inhibitory effects were observed for this condition.

With RSV-B/Wash/18537/62 at 0.13 TCID₅₀/mL, competitive inhibitory effects were observed in the presence of Flu A/Victoria/361/2011 at 1×10⁴ CEID₅₀/mL. No competitive inhibitory effects were observed at a lower concentration of Flu A/Victoria/361/2011 (1×10³ CEID₅₀/mL).

With RSV-B/Wash/18537/62 at 0.13 TCID₅₀/mL, competitive inhibitory effects were observed in the presence of Flu B/Mass/2/2012 at 1×10⁴ TCID₅₀/mL. No competitive inhibitory effects were observed at a lower concentration of Flu B/Mass/2/2012 (1×10¹ TCID₅₀/mL).

When the RSV-B/Wash/18537/62 was increased to 1.3 TCID₅₀/mL (˜10×LoD), competitive inhibitory effects were no longer observed with Flu B/Mass/2/2012 at 1×10⁴ TCID₅₀/mL.

With RSV-B/Wash/18537/62 at 0.13 TCID₅₀/mL in the presence of 1×10⁴ copies/mL SARS-CoV-2 virus, no competitive inhibitory effects were observed for this condition.

6.3.6. Potentially Interfering Substances

Potentially interfering substances that could be present in the nasopharynx (or introduced during specimen collection and handling) and interfere with accurate detection of SARS-CoV-2, Flu A, Flu B and RSV were evaluated with select direct testing on the SARS-CoV-2/Flu/RSV multiplex assay and extrapolated from the interference evaluation of the Xpert® Xpress Flu/RSV test.

Potentially interfering substances in the nasal passage and nasopharynx may include, but are not limited to: blood, nasal secretions or mucus, and nasal and throat medications used to relieve congestion, nasal dryness, irritation, or asthma and allergy symptoms, as well as antibiotics and antivirals. Negative samples (N=8) were tested in the presence of each substance to determine the effect on the performance of the sample processing control (SPC). Positive samples (N=8) were tested per substance with viruses spiked at 3× the analytical LoD determined for each strain. Positive samples tested with the SARS-CoV-2/Flu/RSV multiplex assay included one SARS-CoV-2, two influenza A, one influenza B and two RSV (RSV A and RSV B) strains, whereas those tested with the Xpert® Xpress Flu/RSV consisted of six influenza (four influenza A and two influenza B) and four RSV (two RSV A and two RSV B). All results were compared to positive and negative simulated nasal matrix controls. The simulated nasal matrix consisted of 2.5% (w/v) porcine mucin, 1% (v/v) human whole blood in 0.85% sodium chloride (NaCl) formulated in lx PBS solution with 15% glycerol, which was then diluted to a concentration of 2.5% v/v in Universal Transport Medium (UTM). The substances evaluated are listed in Table I with active ingredients and final concentrations tested shown. None of the substances caused interference of the assay performance at the concentrations tested in this study. All positive and negative replicates were correctly identified by the SARS-CoV-2/Flu/RSV multiplex assay and/or Xpert® Xpress Flu/RSV tests.

TABLE I
Potentially Interfering Substances in the SARS-CoV-2/Flu/RSV Multiplex Assay

| Substance/Class | Description/Active Ingredient | Concentration Tested | |
|---|---|---|---|
| Control | Simulated nasal matrix | 100% | (v/v) |
| Beta-adrenergic | Albuterol Sulfate^{a} | 0.83 mg/mL | |
| bronchodilator | | (equivalent to 1 dose per day) | |
| Blood | Blood (Human) | 2% | (v/v) |
| BD Universal Transport | Transport Media | 100% | (v/v) |
| System | | | |
| Remel M4 ® | Transport Media | 100% | (v/v) |
| Remel M4RT ® | Transport Media | 100% | (v/v) |
| Remel M5 ® | Transport Media | 100% | (v/v) |
| Remel M6 ® | Transport Media | 100% | (v/v) |
| Throat lozenges, oral | Benzocaine, Menthol | 1.7 | mg/mL |
| anesthetic and analgesic | | | |
| Mucin^{a} | Purified Mucin protein | 1% | (w/v)^{a,b} |
| | (Bovine or porcine submaxillary gland) | | |
| Antibiotic, nasal ointment | Mupirocin^{a} | 10 | mg/mL |
| Saline Nasal Spray^{a} | Sodium Chloride (0.65%) | 15% | (v/v) |
| Anefrin Nasal Spray | Oxymetazoline, 0.05% | 15% | (v/v) |
| PHNY Nasal Drops | Phenylephrine, 0.5% | 15% | (v/v) |
| Tamiflu anti-viral drugs | Zanamivir^{a} | 7.5 | mg/mL |
| Antibacterial, systemic | Tobramycin | 4 | μg/mL |
| Zicam Nasal Gel | Luffa opperculata, Galphimia glauca, | 15% | (w/v) |
| | Histaminum hydrochloricum Sulfur | | |
| Nasal corticosteroid | Fluticasone Propionate | 5 | μg/mL |

^{a}Substances/active ingredients and concentrations directly evaluated with the SARS-CoV-2/Flu/RSV Multiplex Assay.
^{b}No interference to the Xpert ® Xpress Flu/RSV performance observed at a concentration of 2.5%

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that changes can be made without departing from the spirit and scope of the invention(s).

TABLE OF CERTAIN SEQUENCES

| SEQ ID | | |
|---|---|---|
| NO | Description | Sequence |
| 1 | Flu A 1 | ATGGAGAGAATAAAAGAACTAAGAGATCTAATGTCGCAGTCTCGCACTCGCGAGATACT |
| | polymerase basic | CACTAAGACCACTGTGGACCATATGGCCATAATCAAAAAGTACACGTCAGGAAGGCAGG |
| | 2 (PB2) gene | AGAAGAACCCCGCACTCAGAATGAAATGGATGATGGCAATGAAATACCCAATTACAGCA |
| | (KC471406.1 for | GACAGGAGAATAATGGACATGATTCCAGAGAGGAATGAACAAGGACAAACCCTCTGGAG |
| | Influenza A virus | CAAAACAACCGATGCTGGATCGGACCGTGTGATGGTATCACCCCTGGCCGTAACATGGT |
| | A/Swine/Korea/ | GGAATAGGAATGGCCCAACAACAAGCACAGTTCACTACCCTAAGGTATACAAAACTTAT |
| | CY02-09/2012 | TTCGAAAAAGTCGAAAGGTTAAAACATGGTACCTTTGGCCCTGTCCACTTCAGAAATCA |
| | (H3N2) segment | AGTTAAAATAAGAAGGAGGOTTGACACAAACCCCGGTCATGCAGATCTCAGTGCCAAGG |
| | 1) | AGGCACAGGATGTGATCATGGAAGTTGTTTTCCCAAACGAAGTGGGGGCAAGAATACTG |
| | | ACATCAGAGTCACAGCTGACAATAACAAAAGAAAAGAAAGAAGAGCTCCAGGATTGTAA |
| | | AATTGCTCCCTTGATGGTGGCATACATGCTAGAAAGAGAATTGGTTCGTAAGACGAGGT |
| | | TTCTTCCGGTGGCTGGTGGAACAAGCAGTGTTTATATTGAAGTGCTGCACTTAACTCAG |
| | | GGAACATGTTGGGAACAAATGTACACTCCAGGAGGAGAAGTGAGAAATGATGATGTTGA |
| | | CCAAAGTTTGATTATCGCCGCTAGAAACATAGTAAGAAGAGCAGCAGTGTCAGCAGACC |
| | | CATTAGCATCTCTCTTGGAAATGTGCCACAGCACACAAATTGGAGATGGAGAGAATAAA |
| | | AGAACTAAGAGATCTAATGTCGCAGTCTCGCACTCGCGAGATACTCACTAAGACCACTG |
| | | TGGACCATATGGCCATAATCAAAAAGTACACGTCAGGAAGGCAGGAGAAGAACCCCGCA |
| | | CTCAGAATGAAATGGATGATGGCAATGAAATACCCAATTACAGCAGACAGGAGAATAAT |
| | | GGACATGATTCCAGAGAGGAATGAACAAGGACAAACCCTCTGGAGCAAAACAACCGATG |
| | | CTGGATCGGACCGTGTGATGGTATCACCCCTGGCCGTAACATGGTGGAATAGGAATGGC |
| | | CCAACAACAAGCACAGTTCACTACCCTAAGGTATACAAAACTTATTTCGAAAAAGTCGA |
| | | AAGGTTAAAACATGGTACCTTTGGCCCTGTCCACTTCAGAAATCAAGTTAAAATAAGAA |
| | | GGAGGGTTGACACAAACCCCGGTCATGCAGATCTCAGTGCCAAGGAGGCACAGGATGTG |
| | | ATCATGGAAGTTGTTTTCCCAAACGAAGTGGGGGCAAGAATACTGACATCAGAGTCACA |
| | | GCTGACAATAACAAAAGAAAAGAAAGAAGAGCTCCAGGATTGTAAAATTGCTCCCTTGA |
| | | TGGTGGCATACATGCTAGAAAGAGAATTGGTTCGTAAGACGAGGTTTCTTCCGGTGGCT |
| | | GGTGGAACAAGCAGTGTTTATATTGAAGTGCTGCACTTAACTCAGGGAACATGTTGGGA |
| | | ACAAATGTACACTCCAGGAGGAGAAGTGAGAAATGATGATGTTGACCAAAGTTTGATTA |
| | | TCGCCGCTAGAAACATAGTAAGAAGAGCAGCAGTGTCAGCAGACCCATTAGCATCTCTC |
| | | TTGGAAATGTGCCACAGCACACAAATTGGAGGAATAAGGATGATGGACATCCTTAGACA |
| | | GAACCCAACGGAGGAACAAGCCGTAGACATATGCAAGGCAGCAATGGGGCTGAGGATTA |
| | | GCTCCTCTTTCAGCTTTGGTGGGTTCACCTTCAAAAGGACAAGCGGATCATCTGTTAAG |
| | | AAAGAAGAAGAAGTGCTCACGGGCAACCTCCAAACACTGAAAATAAGAGTACATGAAGG |
| | | ATATGAGGAATTCACAATGGTCGGGAGAAGAGCAACAGCTATTCTCAGAAAAGCAACCA |
| | | GGAGATTGATCCAGTTAATAGTAAGTGGAAGAGACGATCAATCAATTGCTGAGGCAATA |
| | | ATTGTGGCCATGGTATTTTCACAAGAGGATTGCATGATCAAAGCAGTTAGGGGCGATCT |
| | | GAACTTTGTCAATAGGGCAAACCAGCGACTGAATCCCATGCACCAACTCTTGAGGCATT |
| | | TCCAAAAGGATGCAAAAGTGCTTTTCCAGAACTGGGGGATTGAACCCATCGACAGTGTA |
| | | ATGGGAATGATCGGAATATTGCCTGATATGACCCCAAGCACGGAAATGTCACTGAGAGG |
| | | TATAAGAGTCAGCAAAATGGGAGTAGATGAATATTCCAGTACGGAGAGAGTGGTAGTGA |
| | | GCATTGACCGATTTTTGAGAGTTCGGGATCAACGAGGGAACGTACTATTGTCCCCCGAA |
| | | GAGGTCAGCGAGACACAGGGAACTGAGAAATTGACCATAACTTATTCGTCATCAATGAT |
| | | GTGGGAGATCAATGGTCCTGAGTCAGTGCTGGTCAACACTTATCAATGGATCATAAGGA |
| | | ACTGGGAAAGCTTGAAAATTCAATGGTCACAGGATCCCACGATGTTATACAACAAAATG |
| | | GAATTTGAACCATTCCAGTCTCTTGTCCCTAAGGCAACCAGAAGTCGTTACAGTGGATT |
| | | CGTGAGGACACTGTTCCAGCAAATGCGGGATGTGCTTGGAACATTTGATACTGTCCAAA |
| | | TAATAAAGCTTCTCCCCTTTGCTGCAGCTCCACCGGAACAGAGTAGGATGCAGTTCTCC |
| | | TCGCTGACTGTGAATGTAAGAGGATCAGGGCTGAGGATACTGGTAAGAGGCAATTCTCC |
| | | AGTGTTCAATTACAATAAAGCAACCAAAAGGCTTACAATTCTTGGAAAAGATGCAGGTG |
| | | CATTGACTGAAGATCCAGATGAAGGCACAGCTGGAGTGGAGTCTGCTGTCCTGAGGGGA |
| | | TTCCTCATTTTGGGTAAAGAAGACAAGAGATATGGCCCAGCATTAAGCATCAATGAACT |
| | | GAGCAATCTTGCAAAAGGAGAGAAGGCTAATGTGCTAATTGGGCAAGGAGACGTGGTGT |
| | | TGGTAATGAAACGGAAACGGGACTCTAGCATACTTACTGACAGCCAGACAGCGACCAAA |
| | | AGGATTCGGATGGCCATCAATTAG |
| 2 | Flu A 1 | ATGGAAGACTTTGTGCGACAATGCTTCAATCCGATGATCGTCGAGCTTGCGGAAAAGGC |
| | polymerase acidic | AATGAAAGAATATGGGGAAGATCCGAAAATCGAAACTAACAAGTTTGCTGCAATATGCA |
| | (PA) gene | CACATTTGGAAGTTTGTTTCATGTATTCGGATTTCCATTTCATCGACGAACGGGGTGAA |
| | (consensus | TCAATAATTGTAGAATCTGGTGACCCGAATGCACTATTGAAGCACCGATTTGAGATAAT |
| | sequence, | TGAAGGAAGAGACCGAATCATGGCCTGGACAGTGGTGAACAGTATATGTAACACAACAG |
| | KC471368.1 | GGGTAGAGAAGCCTAAATTTCTTCCTGATTTGTATGATTACAAAGAAAACCGGTTCATT |
| | Influenza A virus | GAAATTGGAGTAACACGGAGGGAAGTCCACATATATTACCTAGAGAAAGCCAACAAAAT |
| | A/Swine/Korea/ | AAAATCTGAGAAGACACACATTCATGGAAGACTTTGTGCGACAATGCTTCAATCCGATG |
| | CY01-04/2012 | ATCGTCGAGCTTGCGGAAAAGGCAATGAAAGAATATGGGGAAGATCCGAAAATCGAAAC |
| | (H1N1) segment | TAACAAGTTTGCTGCAATATGCACACATTTGGAAGTTTGTTTCATGTATTCGGATTTCC |
| | 3) | ATTTCATCGACGAACGGGGTGAATCAATAATTGTAGAATCTGGTGACCCGAATGCACTA |
| | | TTGAAGCACCGATTTGAGATAATTGAAGGAAGAGACCGAATCATGGCCTGGACAGTGGT |
| | | GAACAGTATATGTAACACAACAGGGGTAGAGAAGCCTAAATTTCTTCCTGATTTGTATG |
| | | ATTACAAAGAAAACCGGTTCATTGAAATTGGAGTAACACGGAGGGAAGTCCACATATAT |
| | | TACCTAGAGAAAGCCAACAAAATAAAATCTGAGAAGACACACATTCACATCTTTTCATT |
| | | CACTGGAGAGGAGATGGCCACCAAAGCAGACTACACCCTTGACGAAGAGAGCAGGGCAA |
| | | GAATCAAAACTAGGCTTTTCACTATAAGACAAGAAATGGCCAGTAGGAGTCTATGGGAT |
| | | TCCTTTCGTCAATCCGAAAGAGGCGAAGAGACAATTGAAGAAAAATTTGAGATTACAGG |
| | | AACTATGCGCAAGCTTGCCGACCAAAGTCTCCCACCGAACTTCTCCAGCCTTGAAAACT |
| | | TTAGAGCCTATGTAGATGGATTCGAGCCGAACGGCTGCATTGAGGGCAAGCTTTCCCAA |
| | | ATGTCAAAGGAAGTGAACGCCAAAATTGAACCATTCTTGAGGACGACACCACGCCCCCT |
| | | CAGATTGCCTGATGGGCCTCTTTGCCATCAGCGGTCAAAGTTCCTGCTGATGGATGCTC |
| | | TGAAATTAAGTATTGAAGACCCGAGTCACGAGGGAGAGGGAATACCACTATATGATGCA |
| | | ATCAAATGCATGAAGACATTCTTTGGCTGGAAAGAGCCTAACATAGTCAAACCACATAA |
| | | GAAAGGCATAAATCCCAATTACCTTATGGCTTGGAAGCAGGTGCTAACAGAGCTACAGG |
| | | ACATTGAAAATGAAGAGAAGATCCCAAGGACAAAGAACATGAAGAGAACAAGCCAATTG |
| | | AAGTGGGCACTCGGTGAAAATATGGCACCAGAAAAAGTAGACTTTGATGACTGCAAAGA |
| | | TGTTGGAGACCTTAAACAGTATGACAGTGATGAGCCAGAGCCCAGATCTCTAGCAAGCT |
| | | GGGTCCAAAATGAATTCAATAAGGCATGTGAATTGACTGATTCAAGCTGGATAGAACTT |
| | | GATGAAATAGGAGAAGATGTTGCCCCGATTGAACATATCGCAAGCATGAGGAGGAACTA |
| | | TTTTACAGCAGAAGTGTCCCACTGCAGGGCTACTGAATACATAATGAAGGGAGTGTACA |
| | | TAAATACGGCCTTGCTCAATGCATCCTGTGCAGCCATGGATGACTTTCAGCTGATCCCA |
| | | ATGATAAGCAAATGTAGGACCAAAGAAGGAAGACGGAAAACAAACCTGTATGGGTTCAT |
| | | TATAAAAGGAAGGTCTCATTTGAGAAATGATACTGATGTGGTGAACTTTGTAAGTATGG |
| | | AGTTCTCACTCACTGACCCGAGACTGGAGCCACACAAATGGGAAAAATACTGTGTTCTT |
| | | GAAATAGGAGACATGCTCTTGAGGACTGCGATAGGCCAAGTGTCGAGGCCCATGTTCCT |
| | | ATATGTGAGAACCAATGGAACCTCCAAGATCAAGATGAAATGGGGCATGGAAATGAGGC |
| | | GCTGCCTTCTTCAGTCCCTTCAGCAGATTGAGAGCATGATTGAGGCCGAGTCTTCTGTC |
| | | AAAGAGAAAGACATGACCAAGGAATTCTTTGAAAACAAATCAGAAACATGGCCAATCGG |
| | | AGAGTCACCCAGAGGAGTGGAGGAAGGCTCTATTGGGAAAGTGTGCAGGACCTTACTGG |
| | | CAAAATCTGTGTTCAACAGTCTATATGCGTCTCCACAACTTGAGGGGTTTTCGGCTGAA |
| | | TCGAGAAAATTGCTTCTCATTGTTCAGGCACTTAGGGACAACCTGGAACCTGGAACCTT |
| | | CGATCTTGGGGGGCTATATGAAGCAATCGAGGAGTGCCTGATTAATGATCCCTGGGTTT |
| | | TGCTTAATGCATCTTGGTTCAACTCCTTCCTCACACATGCACTGAAGTAG |
| 3 | Flu A 1 matrix | ATGAGTCTTCTAACCGAGGTCGAAACGTACGTTCTTTCTATCATACCGTCAGGCCCCCT |
| | protein (MP) gene | CAAAGCCGAGATCGCGCAGAGACTGGAAAGTGTCTTTGCAGGAAAGAACACAGATCTTG |
| | (consensus | AGGCTCTCATGGAATGGCTAAAGACAAGACCAATCTTGTCACCTTTGACTAAGGGAATT |
| | sequence | TTAGGATTTGTGTTCACGCTCACCGTGCCCAGTGAGCGAGGACTGCAGCGTAGACGCTT |
| | KC951136.1 of | TGTCCAAAATGCCCTAAATGGGAATGGGGACCCAAACAACATGGATAGAGCAGTTAAAC |
| | segment 7 from | TATACAAGAAGCTCAAAAGAGAAATAACGTTCCATGGGGCCAAGGAGGTGTCACTAAGC |
| | A/Swine/ | TATTCAACTGGTGCACTTGCCAGTTGCATGGGCCTCATATACAACAGGATGGGAACAGT |
| | Pennsylvania/ | GACCACAGAAGCTGCTTTTGGTCTAGTGTGTGCCACTTGTGAACAGATTGCTGATTCAC |
| | A01432652/2013 | AGCATCGGTCTCACAGACAGATGGCTACTACCACCAATCCACTAATCAGGCATGAGAAC |
| | (H3N2)) | AGAATGGTGCTGGCTAGCACTACGGCAAAGGCTATGGAACAGATGGCTGGATCGAGTGA |
| | | ACAGGCAGCGGAGGCCATGGAGGTTGCTAATCAGACTAGGCAGATGGTACATGCAATGA |
| | | GAACTATTGGGACTCATCCTAGCTCCAGTACTGGTCTGAAAGATGACCTTCTTGAAAAT |
| | | TTGCAGGCCTACCAGAAGCGAATGGGAGTGCAGATGCAGCGATTCAAGTGATCCTCTCG |
| | | CCATTGCAGCAAATATCATTGGGATCTTGCACCTGATATTGTGGATTACTGATCGTCTT |
| | | TTTTTCAAATGTATTTATCGTCGCTTTAAATACGGTTTGAAAAGAGGGCCTTCTACAGA |
| | | AGGAGTGCCTGAGTCCATGAGGGAAGAATATCAACAGGAACAGCAGAGTGCTGTGGATG |
| | | TTGACGATGGTCATTTTGTCAACATAGAGCTAGAGTAA |
| 4 | Flu A 2 1 matrix | CGTACGTTCTATCTATCATTCCATCAGGCCCCCTCAAAGCCGAGATCGCGCAGAGACTT |
| | protein (MP) gene | GAGGATGTTTTTGCAGGGAAGAACGCAGATCTCGAGGCTCTCATGGAGTGGATAAAGAC |
| | (consensus | AAGACCAATCCTGTCACCTCTGACTAAGGGGATTTTAGGGTTTGTGTTCACGCTCACCG |
| | sequence, | TGCCCAGTGAGCGAGGACTGCAGCGTAGACGGTTTGTCCAAAACGCCCTAAATGGGAAT |
| | KF018056.1 | GGAGACCCAAACAACATGGACAAGGCAGTTAAATTATACAAGAAACTGAAGAGGGAAAT |
| | Influenza A virus | GACATTCCATGGAGCAAAGGAAGTTGCACTCAGTTACTCAACTGGTGCGCTTGCCAGCT |
| | A/Taiwan/ | GCATGGGTCTCATATACAACAGGATGGGGACAGTAACTGCAGAAGGGGCTCTTGGATTG |
| | T02081/2013 | GTATGTGCCACTTGTGAGCAGATTGCTGACGCACAACATCGGTCCCACAGGCAGATGGC |
| | (H7N9) segment | AACTACTACCAACCCACTAATTAGGCATGAGAATAGAATGGTACTAGCCAGTACTACGG |
| | 7) | CTAAGGCTATGGAGCAGATGGCTGGATCAAGTGAACAGGCAGCGGAAGCCATGGAAGTT |
| | | GCAAGCCAGGCTAGGCAAATGGTGCAGGCTATGAGAACAGTCGGGACTCACCCTAACTC |
| | | CAGTACAGGTCTAAAGGATGATCTTATTGAAAATTTGCAGGCTTACCAGAACCGGATGG |
| | | GAGTGCAACTGCAGCGGTTCAAGTGATCCTCTCGTTGTTGCAGCTAACATTATTGGGAT |
| | | ATTGCACTTGATATTGTGGATTCTTGATCGTCTTTTCTTCAAATGCATTTATCGTCGCT |
| | | TTAAATACGGTTTGAAAAGAGGGCCTTCTACGGAAGGAATGCCTGAGTCTATGAGGGAA |
| | | GAATATCGGCAGGAACAGCAGAATGCTGTGGATGTTGACGATGGTC |
| 5 | Flu A 3 | ATGAACACTCAAATCCTGGTATTCGCTCTGATTGCGATCATTCCAACAAATGCAGACAA |
| | haemagglutinin | AATCTGCCTCGGACATCATGCCGTGTCAAACGGAACCAAAGTAAACACATTAACTGAAA |
| | (HA) gene | GAGGAGTGGAAGTCGTCAATGCAACTGAAACAGTGGAACGAACAAACATCCCCAGGATC |
| | (consensus | TGCTCAAAAGGGAAAATGACAGTTGACCTCGGTCAATGTGGACTCCTGGGGACAATCAC |
| | sequence, | TGGACCACCTCAATGTGACCAATTCCTAGAATTTTCAGCCGATTTAATTATTGAGAGGC |
| | KC896763.1 | GAGAAGGAAGTGATGTCTGTTATCCTGGGAAATTCGTGAATGAGGAAGCTCTGAGGCAA |
| | Influenza A virus | ATACTCAGAGAATCAGGCGGAATTGACAAGGAAGCAATGGGATTCACATACAGTGGAAT |
| | A/Nanjing/ | AAGAACTAATGGAGCAACCAGTGCATGTAGGAGATCAGGATCTTCATTCTATGCAGAAA |
| | 2913/2013 | TGAAATGGCTCCTGTCAAACACAGATAATGCTGCATTCCCGCAGATGACTAAGTCATAT |
| | (H7N9) segment | AAAAATACAAGAAAAAGCCCAGCTCTAATAGTATGGGGGATCCATCATTCCGTATCAAC |
| | 4) | TGCAGAGCAAACCAAGCTATATGGGAGTGGAAACAAACTGGTGACAGTTGGGAGTTCTA |
| | | ATTATCAACAATCTTTTGTACCGAGTCCAGGAGCGAGACCACAAGTTAATGGTCTATCT |
| | | GGAAGAATTGACTTTCATTGGCTAATGCTAAATCCCAATGATACAGTCACTTTCAGTTT |
| | | CAATGGGGCTTTCATAGCTCCAGACCGTGCAAGCTTCCTGAGAGGAAAATCTATGGGAA |
| | | TCCAGAGTGGAGTACAGGTTGATGCCAATTGTGAAGGGGACTGCTATCATAGTGGAGGG |
| | | ACAATAATAAGTAACTTGCCATTTCAGAACATAGATAGCAGGGCAGTTGGAAAATGTCC |
| | | GAGATATGTTAAGCAAAGGAGTCTGCTGCTAGCAACAGGGATGAAGAATGTTCCTGAGA |
| | | TTCCAAAGGGAAGAGGCCTATTTGGTGCTATAGCGGGTTTCATTGAAAATGGATGGGAA |
| | | GGCCTAATTGATGGTTGGTATGGTTTCAGACACCAGAATGCACAGGGAGAGGGAACTGC |
| | | TGCAGATTACAAAAGCACTCAATCGGCAATTGATCAAATAACAGGAAAATTAAACCGGC |
| | | TTATAGAAAAAACCAACCAACAATTTGAGTTGATAGACAATGAATTCAATGAGGTAGAG |
| | | AAGCAAATCGGTAATGTGATAAATTGGACCAGAGATTCTATAACAGAAGTGTGGTCATA |
| | | CAATGCTGAACTCTTGGTAGCAATGGAGAACCAGCATACAATTGATCTGGCTGATTCAG |
| | | AAATGGACAAACTGTACGAACGAGTGAAAAGACAGCTGAGAGAGAATGCTGAAGAAGAT |
| | | GGCACTGGTTGCTTTGAAATATTTCACAAGTGTGATGATGACTGTATGGCCAGTATTAG |
| | | AAATAACACCTATGATCACAGCAAATACAGGGAAGAGGCAATGCAAAATAGAATACAGA |
| | | TTGACCCAGTCAAACTAAGCAGCGGCTACAAAGATGTGATACTTTGGTTTAGCTTCGGG |
| | | GCATCATGTTTCATACTTCTAGCCATTGTAATGGGCCTTGTCTTCATATGTGTAAAGAA |
| | | TGGAAACATGCGGTGCACTATTTGTATATAA |
| 6 | Flu B matrix | ATGTCGCTGTTTGGAGACACAATTGCCTACCTGCTTTCATTGACAGAGGATGGAGAAGG |
| | protein (MP) gene | CAAAGCAGAACTAGCAGAAAAATTACACTGTTGGTTTGGTGGGAAAGAATTTGACCTAG |
| | (consensus | ACTCTGCCTTGGAATGGATAAAAAACAAAAGATGCTTAACTGATATACAAAAAGCACTA |
| | sequence, | ATTGGTGCCTCTATATGCTTTTTAAAACCCAAAGACCAGGAAAGAAAAAGAAGATTCAT |
| | KC814126.1 | CACAGAGCCCTTATCAGGAATGGGAACAACAGCAACAAAAAAGAAAGGCCTGATTCTGG |
| | Influenza B virus | CTGAGAGAAAAATGAGAAGATGTGTGAGCTTTCATGAAGCATTTGAAATAGCAGAAGGC |
| | B/Utah/03/2011 | CATGAAAGCTCAGCGCTACTATACTGTCTCATGGTCATGTACCTGAATCCTGGAAATTA |
| | segment 7 | TTCAATGCAAGTAAAACTAGGAACGCTCTGTGCTTTATGCGAGAAACAAGCATCACATT |
| | | CACACAGGGCTCATAGCAGAGCAGCGAGATCTTCAGTGCCTGGAGTGAGACGAGAAATG |
| | | CAGATGGTCTCAGCTATGAACACAGCAAAAACAATGAATGGAATGGGAAAAGGAGAAGA |
| | | CGTCCAAAAGCTGGCAGAAGAGTTGCAAAGCAACATTGGAGTGCTGAGATCTCTTGGAG |
| | | CAAGCCAAAAGAATGGGGAAGGGATTGCAAAGGATGTAATGGAAGTGCTAAAGCAGAGC |
| | | TCCATGGGAAATTCAGCTCTTGTGAAGAAATATCTATAATGCTCGAACCATTTCAGATT |
| | | CTTACAATTTGTTCTTTTATCTTATCAGCTCTCCATTTCATGGCTTGGACAATAGGGCA |
| | | TTTGAATCAAATAAAAAGAGGAATAAACATGAAAATACGAATAAAAGGTCCAAACAAAG |
| | | AGACAATAAACAGAGAGGTATCAATTTTGAGACACAGTTACCAAAAAGAAATCCAGGCC |
| | | AAAGAAACAATGAAGGAAGTACTCTCTGACAACATGGAGGTATTGAATGACCACATAAT |
| | | AATTGAGGGGCTTTCTGCCGAAGAGATAATAAAAATGGGTGAAACAGTTTTGGAGATAG |
| | | AAGAATTGCATTAAATTCAATTTTACTATATTTCTTACTATGCATTTAAGCAAATTGTA |
| | | ATCAATGTCAGCAAATAA |
| 7 | Flu B | ATGGCGAACAACAACATGACCACAACACAAATTGAGGTGGGTCCGGGAGCAACCAATGC |
| | nonstructural | CACCATAAACTTTGAAGCAGGAATTCTGGAGTGCTATGAAAGGCTTTCATGGCAAAGAG |
| | (NS) gene | CCCTTGACTACCCCGGTCAAGACCGCCTAAACAGACTAAAGAGAAAATTAGAGTCAAGA |
| | (consensus | ATAAAGACTCACAACAAAAGTGAGCCTGAAAGTAAAAGGATGTCCCTTGAAGAGAGAAA |
| | sequence | AGCAATTGGAGTAAAAATGATGAAAGTACTCCTATTTATGAATCCGTCTGCTGGAATTG |
| | KC892145.1 | AAGGGTTTGAGCCATACTGTATGAACAGTTCCTCAAATAGCAACTGTACGAAATACAAT |
| | Influenza B virus | TGGACCGATTACCCTTCAACACCAGAGAGGTGCCTTGATGACATAGAGGAAGAACCAGA |
| | B/Califomia/03/ | GGATGTTGATGGCCCAACTGAAATAGTATTAAGGGACATGAACAACAAAGATGCAAGGC |
| | 2012 segment 8 | AAAAGATAAAGGAGGAAGTAAACACTCAGAAAGAAGGGAAGTTCCGTTTGACAATAAAA |
| | | AGGGATATGCGTAATGTATTGTCCTTGAGAGTGTTGGTAAATGGAACATTCCTCAAACA |
| | | CCCCAATGGATACAAGTCCTTATCAACTCTGCATAGATTGAATGCATATGACCAGAGTG |
| | | GAAGGCTTGTTGCTAAACTTGTTGCCACTGATGATCTTACAGTGGAGGATGAAGAAGAT |
| | | GGCCATCGGATCCTCAACTCACTCTTCGAGCGTCTTAATGAAGGACATTCAAAGCCAAT |
| | | TCGAGCAGCTGAAACTGCGGTGGGAGTCTTATCCCAATTTGGTCAAGAGCACCGATTAT |
| | | CACCAGAAGAGGGAGACAATTAGACTGGTCACGGAAGAACTTTATCTTTTAAGTAAAAG |
| | | AATTGATGATAACATACTATTCCACAAAACAGTGATAGCTAACAGCTCCATAATAGCTG |
| | | ACATGGTTGTATCATTATCATTATTAGAAACATTGTATGAAATGAAGGATGTGGTTGAA |
| | | GTGTACAGCAGGCAGTGCTTGTGAATTTAAAATAAAAATCCTGTTACTACT |
| 8 | Flu A 1 PB2 | AAACGGGACTCTAGCATACTTACTGACAGCCAGACAGCGACCAAAAGGATTCGGATGGC |
| | amplicon | CATCAATTA |
| 9 | Flu A 1 PA | ATCTTGGGGGGCTATATGAAGCAATCGAGGAGTGCCTGATTAATGATCCCTGGGTTTTG |
| | amplicon | CTTAATGCATCTTGGTTCAACTCCTTCCT |
| 10 | Flu A 1 MP | TTCTAACCGAGGTCGAAACGTACGTTCTTTCTATCATACCGTCAGGCCCCCTCAAAGCC |
| | amplicon | GAGATCGCGCAGAGACTGGAAAGTGTCTTTGCAGGAAAGAACACAGATCTTGAGGCTCT |
| | | CATGGAATGGCTAAAGACAAGACCAAT |
| 11 | Flu A 2 MP | CAAGACCAATCCTGTCACCTCTGACTAAGGGGATTTTAGGGTTTGTGTTCACGCTCACC |
| | amplicon | GTGCCCAGTGAGCGAGGACTGCAGCGTAGACG |
| 12 | Flu A 3 HA | GAAATGAAATGGCTCCTGTCAAACACAGATAATGCTGCATTCCCGCAGATGACTAAGTC |
| | amplicon | ATATAAAAATACAAGAAAAAGC |
| 13 | Flu B MP | TTTGGAGACACAATTGCCTACCTGCTTTCATTGACAGAGGATGGAGAAGGCAAAGCAGA |
| | amplicon | ACTAGCAGAAAAATTACACTGTTGGTTTGGTGGGAAAGAATTTGACCT |
| 14 | Flu B NS | GATGGCCATCGGATCCTCAACTCACTCTTCGAGCGTCTTAATGAAGGACATTCAAAGCC |
| | amplicon | AATTCGAGCAGCTGAAACTGCGGTGGGAGTCTTATCCCAATTTGGTCAAGAGC |
| 15 | RSV A amplicon | TACACTCAACAAAGATCAACTTCTGTCATCCAGCAAATACACCATCCAACGGAGCACAG |
| | | GAGATAGTATTGATACTCCTAATTATGATGTGCAGAAACACATCAACAAGTTATGTGGC |
| | | ATG |
| 16 | RSV B amplicon | CATTAAATAAGGATCAGCTGCTGTCATCCAGCAAATACACTATTCAACGTAGTACAGGA |
| | | GATAATATTGACACTCCCAATTATGATGTGCAAAAACACCTAAACAAACTATGTGGTAT |
| | | GC |
| 44 | SARS-CoV-2 E | ATGTACTCATTCGTTTCGGAAGAGACAGGTACGTTAATAGTTAATAGCGTACTTCTTTT |
| | gene 26249- | TCTTGCTTTCGTGGTATTCTTGCTAGTTACACTAGCCATCCTTACTGCGCTTCGATTGT |
| | 26476 nt of | GTGCGTACTGCTGCAATATTGTTAACGTGAGTCTTGTAAAACCTTCTTTTTACGTTTAC |
| | MT276598 | TCTCGTGTTAAAAATCTGAATTCTTCTAGAGTTCCTGATCTTCTGGTCTAA |
| 45 | SARS-CoV-2 N2 | AACTAAAATGTCTGATAATGGACCCCAAAATCAGCGAAATGCACCCCGCATTACGTTTG |
| | gene 28278- | GTGGACCCTCAGATTCAACTGGCAGTAACCAGAATGGAGAACGCAGTGGGGCGCGATCA |
| | 29537 nt of | AAACAACGTCGGCCCCAAGGTTTACCCAATAATACTGCGTCTTGGTTCACCGCTCTCAC |
| | MT276598 | TCAACATGGCAAGGAAGACCTTAAATTCCCTCGAGGACAAGGCGTTCCAATTAACACCA |
| | | ATAGCAGTCCAGATGACCAAATTGGCTACTACCGAAGAGCTACCAGACGAATTCGTGGT |
| | | GGTGACGGTAAAATGAAAGATCTCAGTCCAAGATGGTATTTCTACTACCTAGGAACTGG |
| | | GCCAGAAGCTGGACTTCCCTATGGTGCTAACAAAGACGGCATCATATGGGTTGCAACTG |
| | | AGGGAGCCTTGAATACACCAAAAGATCACATTGGCACCCGCAATCCTGCTAACAATGCT |
| | | GCAATCGTGCTACAACTTCCTCAAGGAACAACATTGCCAAAAGGCTTCTACGCAGAAGG |
| | | GAGCAGAGGCGGCAGTCAAGCCTCTTCTCGTTCCTCATCACGTAGTCGCAACAGTTCAA |
| | | GAAATTCAACTCCAGGCAGCAGTAAACGAACTTCTCCTGCTAGAATGGCTGGCAATGGC |
| | | GGTGATGCTGCTCTTGCTTTGCTGCTGCTTGACAGATTGAACCAGCTTGAGAGCAAAAT |
| | | GTCTGGTAAAGGCCAACAACAACAAGGCCAAACTGTCACTAAGAAATCTGCTGCTGAGG |
| | | CTTCTAAGAAGCCTCGGCAAAAACGTACTGCCACTAAAGCATACAATGTAACACAAGCT |
| | | TTCGGCAGACGTGGTCCAGAACAAACCCAAGGAAATTTTGGGGACCAGGAACTAATCAG |
| | | ACAAGGAACTGATTACAAACATTGGCCGCAAATTGCACAATTTGCCCCCAGCGCTTCAG |
| | | CGTTCTTCGGAATGTCGCGCATTGGCATGGAAGTCACACCTTCGGGAACGTGGTTGACC |
| | | TACACAGGTGCCATCAAATTGGATGACAAAGATCCAAATTTCAAAGATCAAGTCATTTT |
| | | GCTGAATAAGCATATTGACGCATACAAAACATTCCCACCAACAGAGCCTAAAAAGGACA |
| | | AAAAGAAGAAGGCTGATGAAACTCAAGCCTTACCGCAGAGACAGAAGAAACAGCAAACT |
| | | GTGACTCTTCTTCCTGCTGCAGATTTGGATGATTTCTCCAAACAATTGCAACAATCCAT |
| | | GAGCAGTGCTG |
| 46 | SARS-CoV-2 E | TCGGAAGAGACAGGTACGTTAATAGTTAATAGCGTACTTCTTTTTCTTGCTTTCGTGGT |
| | amplicon | ATTCTTGCTAGTTACACTAGCCATCCTTACTGCG |
| 47 | SARS-CoV-2 N2 | TTACAAACATTGGCCGCAAATTGCACAATTTGCCCCCAGCGCTTCAGCGTTCTTCGGAA |
| | amplicon | TGTCGCGC |
| 66 | SARS-CoV-2 E | AGACAGGTACGTTAATAGTTAATAGCGTACTTCTTTTTCTTGCTTTCGTGGTATTCTTG |
| | Alt amplicon | CTAGTTACACTAGCCATCCTTACTGCGCTTCGATTGTGTGCGTACTGCTGCAATAT |
| | (COV2-E-W) | |
| 79 | SARS-CoV-2 E | GCTTTCGTGGTATTCTTGCTAGTTACACTAGCCATCCTTACTGCGCTTCGATTGTGTGC |
| | Alt amplicon | GTACTGC |
| 80 | SARS-CoV-2 | CAAATGTTAAAAACACTATTAGCATAAGCAGTTGTGGCATCTCCTGATGAGGTTCCACC |
| | RDRP amplicon 1 | TGGTTTAACATATAGTGAACCGCCACACATGACCATTTCACTCAATACTTGAGCACACT |
| | | CATTAGCTAATCTATAGAAACGGTGTGACAAGCTACAACACGTTGTATGTTTGCGAGCA |
| | | AGAACAAGTG |
| 81 | SARS-CoV-2 | CTCATTAGCTAATCTATAGAAACGGTGTGACAAGCTACAACACGTTGTATGTTTGCGAG |
| | RDRP amplicon 2 | CAAGAACAAGTG |

1. A method of detecting the presence or absence of influenza A, influenza B, RSV, and SARS-CoV-2 in a biological sample from a subject comprising:
a) contacting a biological sample from the subject with sets of primers that detect an influenza A gene, an influenza B gene, a RSV gene, and a SARS-CoV-2 gene;
b) conducting one or more polymerase chain reaction (PCR); and
c) detecting an amplicon that is produced by the PCR.

2. (canceled)

3. (canceled)

4. The method of claim 1,
a) wherein the set of primers that detects the presence or absence of influenza A comprises at least one of:
i) a set of primers that detects influenza A PB2 selected from:
1) a forward and reverse primer for detecting a sequence of the influenza PB2 gene;
2) a forward primer comprising a region of at least 15 contiguous nucleotides having a sequence that is at least 85% identical to at least 15 contiguous nucleotides of SEQ ID NO: 1, and a reverse primer comprising a region of at least 15 contiguous nucleotides having a sequence that is at least 85% complementary to at least 15 contiguous nucleotides of SEQ ID NO: 1; and
3) a forward primer comprising a region of at least 15 contiguous nucleotides having a sequence that is at least 85% identical to at least 15 contiguous nucleotides of SEQ ID NO: 17, and a reverse primer comprising a region of at least 15 contiguous nucleotides having a sequence that is at least 85% identical to at least 15 contiguous nucleotides of SEQ ID NO: 18;
ii) a set of primers that detects influenza A PA selected from:
1) a forward and reverse primer for detecting a sequence of the influenza PA gene;
2) a forward primer comprising a region of at least 15 contiguous nucleotides having a sequence that is at least 85% identical to at least 15 contiguous nucleotides of SEQ ID NO: 2, and a reverse primer comprising a region of at least 15 contiguous nucleotides having a sequence that is at least 85% complementary to at least 15 contiguous nucleotides of SEQ ID NO: 2; and
3) a forward primer comprising a region of at least 15 contiguous nucleotides having a sequence that is at least 85% identical to at least 15 contiguous nucleotides of SEQ ID NO: 20, and a reverse primer comprising a region of at least 15 contiguous nucleotides having a sequence that is at least 85% identical to at least 15 contiguous nucleotides of SEQ ID NO: 21;
iii) a set of primers that detects influenza A MP selected from:
1) a forward and reverse primer for detecting a sequence of the influenza A MP gene;
2) a forward primer comprising a region of at least 15 contiguous nucleotides having a sequence that is at least 85% identical to at least 15 contiguous nucleotides of SEQ ID NO: 3, and a reverse primer comprising a region of at least 15 contiguous nucleotides having a sequence that is at least 85% complementary to at least 15 contiguous nucleotides of SEQ ID NO: 3; and
3) a forward primer comprising a region of at least 15 contiguous nucleotides having a sequence that is at least 85% identical to at least 15 contiguous nucleotides of SEQ ID NO: 23, and a reverse primer comprising a region of at least 15 contiguous nucleotides having a sequence that is at least 85% identical to at least 15 contiguous nucleotides of SEQ ID NO: 24;
iv) a set of primers that detects avian influenza MP selected from:
1) a forward and reverse primer for detecting a sequence of the avian influenza MP gene;
2) a forward primer comprising a region of at least 15 contiguous nucleotides having a sequence that is at least 85% identical to at least 15 contiguous nucleotides of SEQ ID NO: 4, and a reverse primer comprising a region of at least 15 contiguous nucleotides having a sequence that is at least 85% complementary to at least 15 contiguous nucleotides of SEQ ID NO: 4; and
3) a forward primer comprising a region of at least 15 contiguous nucleotides having a sequence that is at least 85% identical to at least 15 contiguous nucleotides of SEQ ID NO: 26, and a reverse primer comprising a region of at least 15 contiguous nucleotides having a sequence that is at least 85% identical to at least 15 contiguous nucleotides of SEQ ID NO: 27;
b) wherein the set of primers that detects the presence or absence of influenza B comprises at least one of:
i) a set of primers that detects influenza B MP selected from:
1) a forward and reverse primer for detecting a sequence of the influenza B MP gene;
2) a forward primer comprising a region of at least 15 contiguous nucleotides having a sequence that is at least 85% identical to at least 15 contiguous nucleotides of SEQ ID NO: 6, and a reverse primer comprising a region of at least 15 contiguous nucleotides having a sequence that is at least 85% complementary to at least 15 contiguous nucleotides of SEQ ID NO: 6; and
3) a forward primer comprising a region of at least 15 contiguous nucleotides having a sequence that is at least 85% identical to at least 15 contiguous nucleotides of SEQ ID NO: 32, and a reverse primer comprising a region of at least 15 contiguous nucleotides having a sequence that is at least 85% identical to at least 15 contiguous nucleotides of SEQ ID NO: 33;
ii) a set of primers that detects influenza B NS selected from:
1) a forward and reverse primer for detecting a sequence of the influenza B NS gene;
2) a forward primer comprising a region of at least 15 contiguous nucleotides having a sequence that is at least 85% identical to at least 15 contiguous nucleotides of SEQ ID NO: 7, and a reverse primer comprising a region of at least 15 contiguous nucleotides having a sequence that is at least 85% complementary to at least 15 contiguous nucleotides of SEQ ID NO: 7; and
3) a forward primer comprising a region of at least 15 contiguous nucleotides having a sequence that is at least 85% identical to at least 15 contiguous nucleotides of SEQ ID NO: 35, and a reverse primer comprising a region of at least 15 contiguous nucleotides having a sequence that is at least 85% identical to at least 15 contiguous nucleotides of SEQ ID NO: 36;
c) wherein the set of primers that detects the presence or absence of RSV comprises at least one of:
i) a set of primers that detects RSV A selected from:
1) at least one forward and at least one reverse primer for detecting a sequence of the RSV A gene; and
2) at least one forward primer comprising a region of at least 15 contiguous nucleotides having a sequence that is at least 85% identical to at least 15 contiguous nucleotides of SEQ ID NO: 38 and/or SEQ ID NO: 67, and at least one reverse primer comprising a region of at least 15 contiguous nucleotides having a sequence that is at least 85% identical to at least 15 contiguous nucleotides of SEQ ID NO: 39, SEQ ID NO: 68, and/or SEQ ID NO: 69; and
ii) a set of primers that detects RSV B selected from:
1) a forward and reverse primer for detecting a sequence of the RSV B gene; and
2) a forward primer comprising a region of at least 15 contiguous nucleotides having a sequence that is at least 85% identical to at least 15 contiguous nucleotides of SEQ ID NO: 41, and a reverse primer comprising a region of at least 15 contiguous nucleotides having a sequence that is at least 85% identical to at least 15 contiguous nucleotides of SEQ ID NO: 42; and
d) wherein the set of primers that detects the presence or absence of SARS-CoV-2 comprises at least one of:
i) a set of primers that detects SARS-CoV-2 E selected from:
1) a forward and reverse primer for detecting a sequence of the SARS-CoV-2 E gene;
2) a forward primer comprising a region of at least 15 contiguous nucleotides having a sequence that is at least 85% identical to at least 15 contiguous nucleotides of SEQ ID NO: 44, and a reverse primer comprising a region of at least 15 contiguous nucleotides having a sequence that is at least 85% complementary to at least 15 contiguous nucleotides of SEQ ID NO: 44;
3) a forward primer comprising a region of at least 15 contiguous nucleotides having a sequence that is at least 85% identical to at least 15 contiguous nucleotides of SEQ ID NO: 70, and a reverse primer comprising a region of at least 15 contiguous nucleotides having a sequence that is at least 85% identical to at least 15 contiguous nucleotides of SEQ ID NO: 71; and
4) a forward primer comprising a region of at least 15 contiguous nucleotides having a sequence that is at least 85% identical to at least 15 contiguous nucleotides of SEQ ID NO: 48, and a reverse primer comprising a region of at least 15 contiguous nucleotides having a sequence that is at least 85% identical to at least 15 contiguous nucleotides of SEQ ID NO: 49;
ii) a set of primers that detects SARS-CoV-2 N2 selected from:
1) a forward and reverse primer for detecting a sequence of the SARS-CoV-2 N2 gene;
2) a forward primer comprising a region of at least 15 contiguous nucleotides having a sequence that is at least 85% identical to at least 15 contiguous nucleotides of SEQ ID NO: 45, and a reverse primer comprising a region of at least 15 contiguous nucleotides having a sequence that is at least 85% complementary to at least 15 contiguous nucleotides of SEQ ID NO: 45; and
3) a forward primer comprising a region of at least 15 contiguous nucleotides having a sequence that is at least 85% identical to at least 15 contiguous nucleotides of SEQ ID NO: 73 or SEQ ID NO: 51, and a reverse primer comprising a region of at least 15 contiguous nucleotides having a sequence that is at least 85% identical to at least 15 contiguous nucleotides of SEQ ID NO: 52;
iii) a set of primers that detects SARS-CoV-2 RdRP selected from:
1) a forward and at least one reverse primer for detecting a sequence of the SARS-CoV-2 RdRP gene;
2) a forward primer comprising a region of at least 15 contiguous nucleotides having a sequence that is at least 85% identical to at least 15 contiguous nucleotides of SEQ ID NO: 76, and at least one reverse primer comprising a region of at least 15 contiguous nucleotides having a sequence that is at least 85% identical to at least 15 contiguous nucleotides of SEQ ID NO: 61 and/or SEQ ID NO: 78.

5. The method of claim 1,
a) wherein the set of primers that detects the presence or absence of influenza A comprises at least one of:
i) a set of primers that detects influenza A PB2 comprising a forward primer comprising a region of at least 15 contiguous nucleotides having a sequence that is at least 85% identical to at least 15 contiguous nucleotides of SEQ ID NO: 17, and a reverse primer comprising a region of at least 15 contiguous nucleotides having a sequence that is at least 85% identical to at least 15 contiguous nucleotides of SEQ ID NO: 18;
ii) a set of primers that detects influenza A PA comprising a forward primer comprising a region of at least 15 contiguous nucleotides having a sequence that is at least 85% identical to at least 15 contiguous nucleotides of SEQ ID NO: 20, and a reverse primer comprising a region of at least 15 contiguous nucleotides having a sequence that is at least 85% identical to at least 15 contiguous nucleotides of SEQ ID NO: 21;
iii) a set of primers that detects influenza A MP comprising a forward primer comprising a region of at least 15 contiguous nucleotides having a sequence that is at least 85% identical to at least 15 contiguous nucleotides of SEQ ID NO: 23, and a reverse primer comprising a region of at least 15 contiguous nucleotides having a sequence that is at least 85% identical to at least 15 contiguous nucleotides of SEQ ID NO: 24; and
iv) a set of primers that detects avian influenza MP comprising a forward primer comprising a region of at least 15 contiguous nucleotides having a sequence that is at least 85% identical to at least 15 contiguous nucleotides of SEQ ID NO: 26, and a reverse primer comprising a region of at least 15 contiguous nucleotides having a sequence that is at least 85% identical to at least 15 contiguous nucleotides of SEQ ID NO: 27;
b) wherein the set of primers that detects the presence or absence of influenza B comprises at least one of:
i) a set of primers that detects influenza B MP comprising a forward primer comprising a region of at least 15 contiguous nucleotides having a sequence that is at least 85% identical to at least 15 contiguous nucleotides of SEQ ID NO: 32, and a reverse primer comprising a region of at least 15 contiguous nucleotides having a sequence that is at least 85% identical to at least 15 contiguous nucleotides of SEQ ID NO: 33; and
ii) a set of primers that detects influenza B NS comprising a forward primer comprising a region of at least 15 contiguous nucleotides having a sequence that is at least 85% identical to at least 15 contiguous nucleotides of SEQ ID NO: 35, and a reverse primer comprising a region of at least 15 contiguous nucleotides having a sequence that is at least 85% identical to at least 15 contiguous nucleotides of SEQ ID NO: 36;
c) wherein the set of primers that detects the presence or absence of RSV comprises at least one of:
i) a set of primers that detects RSV A comprising at least one forward primer comprising a region of at least 15 contiguous nucleotides having a sequence that is at least 85% identical to at least 15 contiguous nucleotides of SEQ ID NO: 38 and/or SEQ ID NO: 67, and at least one reverse primer comprising a region of at least 15 contiguous nucleotides having a sequence that is at least 85% identical to at least 15 contiguous nucleotides of SEQ ID NO: 39, SEQ ID NO: 68, and/or SEQ ID NO: 69; and
ii) a set of primers that detects RSV B comprising a forward primer comprising a region of at least 15 contiguous nucleotides having a sequence that is at least 85% identical to at least 15 contiguous nucleotides of SEQ ID NO: 41, and a reverse primer comprising a region of at least 15 contiguous nucleotides having a sequence that is at least 85% identical to at least 15 contiguous nucleotides of SEQ ID NO: 42; and
d) wherein the set of primers that detects the presence or absence of SARS-CoV-2 comprises at least one of:
i) a set of primers that detects SARS-CoV-2 E comprising a forward primer comprising a region of at least 15 contiguous nucleotides having a sequence that is at least 85% identical to at least 15 contiguous nucleotides of SEQ ID NO: 70, and a reverse primer comprising a region of at least 15 contiguous nucleotides having a sequence that is at least 85% identical to at least 15 contiguous nucleotides of SEQ ID NO: 71;
ii) a set of primers that detects SARS-CoV-2 N2 comprising a forward primer comprising a region of at least 15 contiguous nucleotides having a sequence that is at least 85% identical to at least 15 contiguous nucleotides of SEQ ID NO: 73, and a reverse primer comprising a region of at least 15 contiguous nucleotides having a sequence that is at least 85% identical to at least 15 contiguous nucleotides of SEQ ID NO: 52; and
iii) a set of primers that detects SARS-CoV-2 RdRP comprising a forward primer comprising a region of at least 15 contiguous nucleotides having a sequence that is at least 85% identical to at least 15 contiguous nucleotides of SEQ ID NO: 76, and at least one reverse primer comprising a region of at least 15 contiguous nucleotides having a sequence that is at least 85% identical to at least 15 contiguous nucleotides of SEQ ID NO: 61 and/or SEQ ID NO: 78.

6. The method of claim 1 wherein:
a) the influenza A PA amplicon comprises a sequence that is at least 85% identical to SEQ ID NO: 9;
b) the influenza A PB2 amplicon comprises a sequence that is at least 85% identical to SEQ ID NO: 8;
c) the influenza A 1MP amplicon comprises a sequence that is at least 85% identical to SEQ ID NO: 10;
d) the avian influenza MP amplicon comprises a sequence that is at least 85% identical to SEQ ID NO: 11;
e) the influenza B 1MP amplicon comprises a sequence that is at least 85% identical to SEQ ID NO: 13;
f) the influenza B NS amplicon comprises a sequence that is at least 85% identical to SEQ ID NO: 14;
g) the RSV A amplicon comprises a sequence that is at least 85% identical to SEQ ID NO: 15;
h) the RSV B amplicon comprises a sequence that is at least 85% identical to SEQ ID NO: 16;
i) the SARS-CoV-2 E amplicon comprises a sequence that is at least 85% identical to SEQ ID NO: 46 or SEQ ID NO: 79;
j) the SARS-CoV-2 N2 amplicon comprises a sequence that is at least 85% identical to SEQ ID NO: 47; and/or
k) the SARS-CoV-2 RdRP amplicon comprises a sequence that is at least 85% identical to SEQ ID NO: 80 and/or SEQ ID NO: 81.

7. The method of claim 6, wherein detecting the amplicons comprises contacting the amplicons with at least one probe selected from an influenza A PA probe, an influenza A PB2 probe, an influenza A MP probe, an avian influenza MP probe, an influenza B MP probe, an influenza B NS probe, an RSV A probe, an RSV B probe, a SARS-CoV-2 E probe, a SARS-CoV-2 N2 probe, and a SARS-CoV-2 RdRP probe.

8. The method of claim 7, wherein
a) the influenza A PA probe comprises a region of at least 15 contiguous nucleotides having a sequence that is at least 85% identical or complementary to at least 15 contiguous nucleotides of SEQ ID NO: 22;
b) the influenza A PB2 probe comprises a region of at least 15 contiguous nucleotides having a sequence that is at least 85% identical or complementary to at least 15 contiguous nucleotides of SEQ ID NO: 19;
c) the influenza A 1VIP probe comprises a region of at least 15 contiguous nucleotides having a sequence that is at least 85% identical or complementary to at least 15 contiguous nucleotides of SEQ ID NO: 25;
d) the avian influenza MP probe comprises a region of at least 15 contiguous nucleotides having a sequence that is at least 85% identical or complementary to at least 15 contiguous nucleotides of SEQ ID NO: 28;
e) the influenza B MP probe comprises a region of at least 15 contiguous nucleotides having a sequence that is at least 85% identical or complementary to at least 15 contiguous nucleotides of SEQ ID NO: 34;
f) the influenza B NS probe comprises a region of at least 15 contiguous nucleotides having a sequence that is at least 85% identical or complementary to at least 15 contiguous nucleotides of SEQ ID NO: 37;
g) the RSV A probe comprises a region of at least 15 contiguous nucleotides having a sequence that is at least 85% identical or complementary to at least 15 contiguous nucleotides of SEQ ID NO: 40;
h) the RSV B probe comprises a region of at least 15 contiguous nucleotides having a sequence that is at least 85% identical or complementary to at least 15 contiguous nucleotides of SEQ ID NO: 43;
i) the SARS-CoV-2 E probe comprises a region of at least 15 contiguous nucleotides having a sequence that is at least 85% identical or complementary to at least 15 contiguous nucleotides of SEQ ID NO: 72, SEQ ID NO: 50 or SEQ ID NO: 56;
j) the SARS-CoV-2 N2 probe comprises a region of at least 15 contiguous nucleotides having a sequence that is at least 85% identical or complementary to at least 15 contiguous nucleotides of SEQ ID NO: 74, SEQ ID NO: 75, and/or SEQ ID NO: 53; and/or
k) the SARS-CoV-2 RdRP probe comprises a region of at least 15 contiguous nucleotides having a sequence that is at least 85% identical or complementary to at least 15 contiguous nucleotides of SEQ ID NO: 77.

9. The method of claim 1, wherein the method further comprises detecting an exogenous control.

10.-12. (canceled)

13. The method of claim 9, wherein the method comprises detecting the presence or absence of at least one influenza A gene, at least one influenza B gene, at least one RSV gene, at least one SARS-CoV-2 gene, and an exogenous control in a single multiplex reaction.

14.-20. (canceled)

21. The method of claim 1, wherein the subject has one or more symptoms of influenza, RSV, and/or COVID-19.

22. (canceled)

23. The method of claim 1, wherein the sample is selected from a nasopharyngeal swab sample, an oropharyngeal sample, a nasal aspirate sample, a nasal or mid-turbinate swab, a nasal aspirate sample, a nasal wash sample, a throat swab sample, a bronchoalveolar lavage sample, a bronchial aspirate sample, a bronchial wash sample, an endotracheal aspirate, an endotracheal wash sample, a tracheal aspirate, a nasal secretion sample, a mucus sample, a sputum sample, a lung tissue samples, a urine sample, a saliva sample, and a fecal sample.

24. (canceled)

25. A composition comprising sets of primers that detect the presence of an influenza A gene, an influenza B gene, a RSV gene, and a SARS-CoV-2 gene,
a. wherein the set of primers that detects the presence or absence of influenza A comprises at least one of:
i. a set of primers that detects influenza A PB2 selected from:
1. a forward and reverse primer for detecting a sequence of the influenza PB2 gene;
2. a forward primer comprising a region of at least 15 contiguous nucleotides having a sequence that is at least 85% identical to at least 15 contiguous nucleotides of SEQ ID NO: 1, and a reverse primer comprising a region of at least 15 contiguous nucleotides having a sequence that is at least 85% complementary to at least 15 contiguous nucleotides of SEQ ID NO: 1; and
3. a forward primer comprising a region of at least 15 contiguous nucleotides having a sequence that is at least 85% identical to at least 15 contiguous nucleotides of SEQ ID NO: 17, and a reverse primer comprising a region of at least 15 contiguous nucleotides having a sequence that is at least 85% identical to at least 15 contiguous nucleotides of SEQ ID NO: 18;
ii. a set of primers that detects influenza A PA selected from:
1. a forward and reverse primer for detecting a sequence of the influenza PA gene;
2. a forward primer comprising a region of at least 15 contiguous nucleotides having a sequence that is at least 85% identical to at least 15 contiguous nucleotides of SEQ ID NO: 2, and a reverse primer comprising a region of at least 15 contiguous nucleotides having a sequence that is at least 85% complementary to at least 15 contiguous nucleotides of SEQ ID NO: 2; and
3. a forward primer comprising a region of at least 15 contiguous nucleotides having a sequence that is at least 85% identical to at least 15 contiguous nucleotides of SEQ ID NO: 20, and a reverse primer comprising a region of at least 15 contiguous nucleotides having a sequence that is at least 85% identical to at least 15 contiguous nucleotides of SEQ ID NO: 21;
iii. a set of primers that detects influenza A MP selected from:
1. a forward and reverse primer for detecting a sequence of the influenza A MP gene;
2. a forward primer comprising a region of at least 15 contiguous nucleotides having a sequence that is at least 85% identical to at least 15 contiguous nucleotides of SEQ ID NO: 3, and a reverse primer comprising a region of at least 15 contiguous nucleotides having a sequence that is at least 85% complementary to at least 15 contiguous nucleotides of SEQ ID NO: 3; and
3. a forward primer comprising a region of at least 15 contiguous nucleotides having a sequence that is at least 85% identical to at least 15 contiguous nucleotides of SEQ ID NO: 23, and a reverse primer comprising a region of at least 15 contiguous nucleotides having a sequence that is at least 85% identical to at least 15 contiguous nucleotides of SEQ ID NO: 24;
iv. a set of primers that detects avian influenza MP selected from:
1. a forward and reverse primer for detecting a sequence of the avian influenza MP gene;
2. a forward primer comprising a region of at least 15 contiguous nucleotides having a sequence that is at least 85% identical to at least 15 contiguous nucleotides of SEQ ID NO: 4, and a reverse primer comprising a region of at least 15 contiguous nucleotides having a sequence that is at least 85% complementary to at least 15 contiguous nucleotides of SEQ ID NO: 4; and
3. a forward primer comprising a region of at least 15 contiguous nucleotides having a sequence that is at least 85% identical to at least 15 contiguous nucleotides of SEQ ID NO: 26, and a reverse primer comprising a region of at least 15 contiguous nucleotides having a sequence that is at least 85% identical to at least 15 contiguous nucleotides of SEQ ID NO: 27;
b. wherein the set of primers that detects the presence or absence of influenza B comprises at least one of:
i. a set of primers that detects influenza B MP selected from:
1. a forward and reverse primer for detecting a sequence of the influenza B MP gene;
2. a forward primer comprising a region of at least 15 contiguous nucleotides having a sequence that is at least 85% identical to at least 15 contiguous nucleotides of SEQ ID NO: 6, and a reverse primer comprising a region of at least 15 contiguous nucleotides having a sequence that is at least 85% complementary to at least 15 contiguous nucleotides of SEQ ID NO: 6; and
3. a forward primer comprising a region of at least 15 contiguous nucleotides having a sequence that is at least 85% identical to at least 15 contiguous nucleotides of SEQ ID NO: 32, and a reverse primer comprising a region of at least 15 contiguous nucleotides having a sequence that is at least 85% identical to at least 15 contiguous nucleotides of SEQ ID NO: 33;
ii. a set of primers that detects influenza B NS selected from:
1. a forward and reverse primer for detecting a sequence of the influenza B NS gene;
2. a forward primer comprising a region of at least 15 contiguous nucleotides having a sequence that is at least 85% identical to at least 15 contiguous nucleotides of SEQ ID NO: 7, and a reverse primer comprising a region of at least 15 contiguous nucleotides having a sequence that is at least 85% complementary to at least 15 contiguous nucleotides of SEQ ID NO: 7; and
3. a forward primer comprising a region of at least 15 contiguous nucleotides having a sequence that is at least 85% identical to at least 15 contiguous nucleotides of SEQ ID NO: 35, and a reverse primer comprising a region of at least 15 contiguous nucleotides having a sequence that is at least 85% identical to at least 15 contiguous nucleotides of SEQ ID NO: 36;
c. wherein the set of primers that detects the presence or absence of RSV comprises at least one of:
i. a set of primers that detects RSV A selected from:
1. at least one forward and at least one reverse primer for detecting a sequence of the RSV A gene; and
2. at least one forward primer comprising a region of at least 15 contiguous nucleotides having a sequence that is at least 85% identical to at least 15 contiguous nucleotides of SEQ ID NO: 38 and/or SEQ ID NO: 67, and at least one reverse primer comprising a region of at least 15 contiguous nucleotides having a sequence that is at least 85% complementary identical to at least 15 contiguous nucleotides of SEQ ID NO: 39, SEQ ID NO: 68, and/or SEQ ID NO: 69; and
ii. a set of primers that detects RSV B selected from:
1. a forward and reverse primer for detecting a sequence of the RSV B gene; and
2. a forward primer comprising a region of at least 15 contiguous nucleotides having a sequence that is at least 85% identical to at least 15 contiguous nucleotides of SEQ ID NO: 41, and a reverse primer comprising a region of at least 15 contiguous nucleotides having a sequence that is at least 85% identical to at least 15 contiguous nucleotides of SEQ ID NO: 42; and
d. wherein the set of primers that detects the presence or absence of SARS-CoV-2 comprises at least one of:
i. a set of primers that detects SARS-CoV-2 E selected from:
1. a forward and reverse primer for detecting a sequence of the SARS-CoV-2 E gene;
2. a forward primer comprising a region of at least 15 contiguous nucleotides having a sequence that is at least 85% identical to at least 15 contiguous nucleotides of SEQ ID NO: 44, and a reverse primer comprising a region of at least 15 contiguous nucleotides having a sequence that is at least 85% complementary to at least 15 contiguous nucleotides of SEQ ID NO: 44;
3. a forward primer comprising a region of at least 15 contiguous nucleotides having a sequence that is at least 85% identical to at least 15 contiguous nucleotides of SEQ ID NO: 70, and a reverse primer comprising a region of at least 15 contiguous nucleotides having a sequence that is at least 85% identical to at least 15 contiguous nucleotides of SEQ ID NO: 71; and
4. a forward primer comprising a region of at least 15 contiguous nucleotides having a sequence that is at least 85% identical to at least 15 contiguous nucleotides of SEQ ID NO: 48, and a reverse primer comprising a region of at least 15 contiguous nucleotides having a sequence that is at least 85% identical to at least 15 contiguous nucleotides of SEQ ID NO: 49;
ii. a set of primers that detects SARS-CoV-2 N2 selected from:
1. a forward and reverse primer for detecting a sequence of the SARS-CoV-2 N2 gene;
2. a forward primer comprising a region of at least 15 contiguous nucleotides having a sequence that is at least 85% identical to at least 15 contiguous nucleotides of SEQ ID NO: 45, and a reverse primer comprising a region of at least 15 contiguous nucleotides having a sequence that is at least 85% complementary to at least 15 contiguous nucleotides of SEQ ID NO: 45; and
3. a forward primer comprising a region of at least 15 contiguous nucleotides having a sequence that is at least 85% identical to at least 15 contiguous nucleotides of SEQ ID NO: 73 or SEQ ID NO: 51, and a reverse primer comprising a region of at least 15 contiguous nucleotides having a sequence that is at least 85% identical to at least 15 contiguous nucleotides of SEQ ID NO: 52
iii. a set of primers that detects SARS-CoV-2 RdRP selected from:
1. a forward and at least one reverse primer for detecting a sequence of the SARS-CoV-2 RdRP gene;
2. a forward primer comprising a region of at least 15 contiguous nucleotides having a sequence that is at least 85% identical to at least 15 contiguous nucleotides of SEQ ID NO: 76, and at least one reverse primer comprising a region of at least 15 contiguous nucleotides having a sequence that is at least 85% identical to at least 15 contiguous nucleotides of SEQ ID NO: 61 and/or SEQ ID NO: 78.

26. The composition of claim 25,
a. wherein the set of primers that detects the presence or absence of influenza A comprises at least one of:
i. a set of primers that detects influenza A PB2 comprising a forward primer comprising a region of at least 15 contiguous nucleotides having a sequence that is at least 85% identical to at least 15 contiguous nucleotides of SEQ ID NO: 17, and a reverse primer comprising a region of at least 15 contiguous nucleotides having a sequence that is at least 85% identical to at least 15 contiguous nucleotides of SEQ ID NO: 18;
ii. a set of primers that detects influenza A PA comprising a forward primer comprising a region of at least 15 contiguous nucleotides having a sequence that is at least 85% identical to at least 15 contiguous nucleotides of SEQ ID NO: 20, and a reverse primer comprising a region of at least 15 contiguous nucleotides having a sequence that is at least 85% identical to at least 15 contiguous nucleotides of SEQ ID NO: 21;
iii. a set of primers that detects influenza AMP comprising a forward primer comprising a region of at least 15 contiguous nucleotides having a sequence that is at least 85% identical to at least 15 contiguous nucleotides of SEQ ID NO: 23, and a reverse primer comprising a region of at least 15 contiguous nucleotides having a sequence that is at least 85% identical to at least 15 contiguous nucleotides of SEQ ID NO: 24; and
iv. a set of primers that detects avian influenza MP comprising a forward primer comprising a region of at least 15 contiguous nucleotides having a sequence that is at least 85% identical to at least 15 contiguous nucleotides of SEQ ID NO: 26, and a reverse primer comprising a region of at least 15 contiguous nucleotides having a sequence that is at least 85% identical to at least 15 contiguous nucleotides of SEQ ID NO: 27;
b. wherein the set of primers that detects the presence or absence of influenza B comprises at least one of:
i. a set of primers that detects influenza B MP comprising a forward primer comprising a region of at least 15 contiguous nucleotides having a sequence that is at least 85% identical to at least 15 contiguous nucleotides of SEQ ID NO: 32, and a reverse primer comprising a region of at least 15 contiguous nucleotides having a sequence that is at least 85% identical to at least 15 contiguous nucleotides of SEQ ID NO: 33; and
ii. a set of primers that detects influenza B NS comprising a forward primer comprising a region of at least 15 contiguous nucleotides having a sequence that is at least 85% identical to at least 15 contiguous nucleotides of SEQ ID NO: 35, and a reverse primer comprising a region of at least 15 contiguous nucleotides having a sequence that is at least 85% identical to at least 15 contiguous nucleotides of SEQ ID NO: 36;
c. wherein the set of primers that detects the presence or absence of RSV comprises at least one of:
i. a set of primers that detects RSV A comprising at least one forward primer comprising a region of at least 15 contiguous nucleotides having a sequence that is at least 85% identical to at least 15 contiguous nucleotides of SEQ ID NO: 38 and/or SEQ ID NO: 67, and at least one reverse primer comprising a region of at least 15 contiguous nucleotides having a sequence that is at least 85% identical to at least 15 contiguous nucleotides of SEQ ID NO: 39, SEQ ID NO: 68, and/or SEQ ID NO: 69; and
ii. a set of primers that detects RSV B comprising a forward primer comprising a region of at least 15 contiguous nucleotides having a sequence that is at least 85% identical to at least 15 contiguous nucleotides of SEQ ID NO: 41, and a reverse primer comprising a region of at least 15 contiguous nucleotides having a sequence that is at least 85% identical to at least 15 contiguous nucleotides of SEQ ID NO: 42; and
d. wherein the set of primers that detects the presence or absence of SARS-CoV-2 comprises at least one of:
i. a set of primers that detects SARS-CoV-2 E comprising a forward primer comprising a region of at least 15 contiguous nucleotides having a sequence that is at least 85% identical to at least 15 contiguous nucleotides of SEQ ID NO: 70, and a reverse primer comprising a region of at least 15 contiguous nucleotides having a sequence that is at least 85% identical to at least 15 contiguous nucleotides of SEQ ID NO: 71;
ii. a set of primers that detects SARS-CoV-2 N2 comprising a forward primer comprising a region of at least 15 contiguous nucleotides having a sequence that is at least 85% identical to at least 15 contiguous nucleotides of SEQ ID NO: 73, and a reverse primer comprising a region of at least 15 contiguous nucleotides having a sequence that is at least 85% identical to at least 15 contiguous nucleotides of SEQ ID NO: 52; and
iii. a set of primers that detects SARS-CoV-2 RdRP comprising a forward primer comprising a region of at least 15 contiguous nucleotides having a sequence that is at least 85% identical to at least 15 contiguous nucleotides of SEQ ID NO: 76, and at least one reverse primer comprising a region of at least 15 contiguous nucleotides having a sequence that is at least 85% identical to at least 15 contiguous nucleotides of SEQ ID NO: 61 and/or SEQ ID NO: 78.

27. The composition of claim 25, further comprising a primer pair for detecting an exogenous control.

28. (canceled)

29. (canceled)

30. The composition of claim 25, further comprising at least one probe selected from an influenza A PA probe, an influenza A PB2 probe, an influenza A MP probe, an avian influenza MP probe, an influenza B MP probe, an influenza B NS probe, an RSV A probe, an RSV B probe, a SARS-CoV-2 E probe, a SARS-CoV-2 N2 probe, and a SARS-CoV-2 RdRP probe.

31. The composition of claim 30, wherein
a. the influenza A PA probe comprises a region of at least 15 contiguous nucleotides having a sequence that is at least 85% identical or complementary to at least 15 contiguous nucleotides of SEQ ID NO: 22;
b. the influenza A PB2 probe comprises a region of at least 15 contiguous nucleotides having a sequence that is at least 85% identical or complementary to at least 15 contiguous nucleotides of SEQ ID NO: 19;
c. the influenza A 1VIP probe comprises a region of at least 15 contiguous nucleotides having a sequence that is at least 85% identical or complementary to at least 15 contiguous nucleotides of SEQ ID NO: 25;
d. the avian influenza MP probe comprises a region of at least 15 contiguous nucleotides having a sequence that is at least 85% identical or complementary to at least 15 contiguous nucleotides of SEQ ID NO: 28;
e. the influenza B MP probe comprises a region of at least 15 contiguous nucleotides having a sequence that is at least 85% identical or complementary to at least 15 contiguous nucleotides of SEQ ID NO: 34;
f. the influenza B NS probe comprises a region of at least 15 contiguous nucleotides having a sequence that is at least 85% identical or complementary to at least 15 contiguous nucleotides of SEQ ID NO: 37;
g. the RSV A probe comprises a region of at least 15 contiguous nucleotides having a sequence that is at least 85% identical or complementary to at least 15 contiguous nucleotides of SEQ ID NO: 40;
h. the RSV B probe comprises a region of at least 15 contiguous nucleotides having a sequence that is at least 85% identical or complementary to at least 15 contiguous nucleotides of SEQ ID NO: 43;
i. the SARS-CoV-2 E probe comprises a region of at least 15 contiguous nucleotides having a sequence that is at least 85% identical or complementary to at least 15 contiguous nucleotides of SEQ ID NO: 72, SEQ ID NO: 50 or SEQ ID NO: 56;
j. the SARS-CoV-2 N2 probe comprises a region of at least 15 contiguous nucleotides having a sequence that is at least 85% identical or complementary to at least 15 contiguous nucleotides of SEQ ID NO: 74, SEQ ID NO: 75, and/or SEQ ID NO: 53; and/or
k. the SARS-CoV-2 RdRP probe comprises a region of at least 15 contiguous nucleotides having a sequence that is at least 85% identical or complementary to at least 15 contiguous nucleotides of SEQ ID NO: 77.

32. The composition of claim 27, further comprising a probe for detecting an exogenous control.

33. The composition of claim 31, wherein each probe comprises a detectable label.

34.-36. (canceled)

37. The composition of claim 25, wherein the composition comprises nucleic acids from a sample from a subject being tested for the presence or absence of influenza, RSV, and/or COVID-19.

38. (canceled)

39. A kit comprising the composition of claim 25.

40.-46. (canceled)

47. A method of detecting the presence or absence of SARS-CoV-2 in a biological sample from a subject and/or determining whether a subject has COVID-19, comprising:
a. contacting a biological sample from the subject with sets of primers that detect a SARS-CoV-2 E, SARS-CoV-2 N2 gene, and/or SARS-CoV-2 RdRP gene;
b. conducting one or more polymerase chain reaction (PCR); and
c. detecting an amplicon that is produced by the PCR;
wherein the set of primers that detects the presence or absence of SARS-CoV-2 E, SARS-CoV-2 N2 gene, and/or SARS-CoV-2 RdRP genes comprises at least one of:
a) a set of primers that detects SARS-CoV-2 E selected from:
i. a forward and reverse primer for detecting a sequence of the SARS-CoV-2 E gene;
ii. a forward primer comprising a region of at least 15 contiguous nucleotides having a sequence that is at least 85% identical to at least 15 contiguous nucleotides of SEQ ID NO: 44, and a reverse primer comprising a region of at least 15 contiguous nucleotides having a sequence that is at least 85% complementary to at least 15 contiguous nucleotides of SEQ ID NO: 44; and
iii. a forward primer comprising a region of at least 15 contiguous nucleotides having a sequence that is at least 85% identical to at least 15 contiguous nucleotides of SEQ ID NO: 70, and a reverse primer comprising a region of at least 15 contiguous nucleotides having a sequence that is at least 85% identical to at least 15 contiguous nucleotides of SEQ ID NO: 71; and
iv. a forward primer comprising a region of at least 15 contiguous nucleotides having a sequence that is at least 85% identical to at least 15 contiguous nucleotides of SEQ ID NO: 48, and a reverse primer comprising a region of at least 15 contiguous nucleotides having a sequence that is at least 85% identical to at least 15 contiguous nucleotides of SEQ ID NO: 49;
b) a set of primers that detects SARS-CoV-2 N2 selected from:
i) a forward and reverse primer for detecting a sequence of the SARS-CoV-2 N2 gene;
ii) a forward primer comprising a region of at least 15 contiguous nucleotides having a sequence that is at least 85% identical to at least 15 contiguous nucleotides of SEQ ID NO: 45, and a reverse primer comprising a region of at least 15 contiguous nucleotides having a sequence that is at least 85% complementary to at least 15 contiguous nucleotides of SEQ ID NO: 45; and
iii) a forward primer comprising a region of at least 15 contiguous nucleotides having a sequence that is at least 85% identical to at least 15 contiguous nucleotides of SEQ ID NO: 73 or SEQ ID NO: 51, and a reverse primer comprising a region of at least 15 contiguous nucleotides having a sequence that is at least 85% identical to at least 15 contiguous nucleotides of SEQ ID NO: 52; and
c) a set of primers that detects SARS-CoV-2 RdRP selected from:
i) a forward and at least one reverse primer for detecting a sequence of the SARS-CoV-2 RdRP gene;
ii) a forward primer comprising a region of at least 15 contiguous nucleotides having a sequence that is at least 85% identical to at least 15 contiguous nucleotides of SEQ ID NO: 76, and at least one reverse primer comprising a region of at least 15 contiguous nucleotides having a sequence that is at least 85% identical to at least 15 contiguous nucleotides of SEQ ID NO: 61 and/or SEQ ID NO: 78.

48.-50. (canceled)


SEQUENCE LISTING (81 sequences)

SEQ ID NO: 1 - DNA, 3151 bp, Influenza A virus
  misc_feature (1)..(3151): Flu A 1 polymerase basic 2 (PB2) gene (KC471406.1 for Influenza A virus A/Swine/Korea/ CY02-09/2012 (H3N2) segment 1)
atggagagaataaaagaactaagagatctaatgtcgcagtctcgcactcgcgagatactcactaagaccactgtggaccatatggccataatcaaaaagtacacgtcaggaaggcaggagaagaaccccgcactcagaatgaaatggatgatggcaatgaaatacccaattacagcagacaggagaataatggacatgattccagagaggaatgaacaaggacaaaccctctggagcaaaacaaccgatgctggatcggaccgtgtgatggtatcacccctggccgtaacatggtggaataggaatggcccaacaacaagcacagttcactaccctaaggtatacaaaacttatttcgaaaaagtcgaaaggttaaaacatggtacctttggccctgtccacttcagaaatcaagttaaaataagaaggagggttgacacaaaccccggtcatgcagatctcagtgccaaggaggcacaggatgtgatcatggaagttgttttcccaaacgaagtgggggcaagaatactgacatcagagtcacagctgacaataacaaaagaaaagaaagaagagctccaggattgtaaaattgctcccttgatggtggcatacatgctagaaagagaattggttcgtaagacgaggtttcttccggtggctggtggaacaagcagtgtttatattgaagtgctgcacttaactcagggaacatgttgggaacaaatgtacactccaggaggagaagtgagaaatgatgatgttgaccaaagtttgattatcgccgctagaaacatagtaagaagagcagcagtgtcagcagacccattagcatctctcttggaaatgtgccacagcacacaaattggagatggagagaataaaagaactaagagatctaatgtcgcagtctcgcactcgcgagatactcactaagaccactgtggaccatatggccataatcaaaaagtacacgtcaggaaggcaggagaagaaccccgcactcagaatgaaatggatgatggcaatgaaatacccaattacagcagacaggagaataatggacatgattccagagaggaatgaacaaggacaaaccctctggagcaaaacaaccgatgctggatcggaccgtgtgatggtatcacccctggccgtaacatggtggaataggaatggcccaacaacaagcacagttcactaccctaaggtatacaaaacttatttcgaaaaagtcgaaaggttaaaacatggtacctttggccctgtccacttcagaaatcaagttaaaataagaaggagggttgacacaaaccccggtcatgcagatctcagtgccaaggaggcacaggatgtgatcatggaagttgttttcccaaacgaagtgggggcaagaatactgacatcagagtcacagctgacaataacaaaagaaaagaaagaagagctccaggattgtaaaattgctcccttgatggtggcatacatgctagaaagagaattggttcgtaagacgaggtttcttccggtggctggtggaacaagcagtgtttatattgaagtgctgcacttaactcagggaacatgttgggaacaaatgtacactccaggaggagaagtgagaaatgatgatgttgaccaaagtttgattatcgccgctagaaacatagtaagaagagcagcagtgtcagcagacccattagcatctctcttggaaatgtgccacagcacacaaattggaggaataaggatgatggacatccttagacagaacccaacggaggaacaagccgtagacatatgcaaggcagcaatggggctgaggattagctcctctttcagctttggtgggttcaccttcaaaaggacaagcggatcatctgttaagaaagaagaagaagtgctcacgggcaacctccaaacactgaaaataagagtacatgaaggatatgaggaattcacaatggtcgggagaagagcaacagctattctcagaaaagcaaccaggagattgatccagttaatagtaagtggaagagacgatcaatcaattgctgaggcaataattgtggccatggtattttcacaagaggattgcatgatcaaagcagttaggggcgatctgaactttgtcaatagggcaaaccagcgactgaatcccatgcaccaactcttgaggcatttccaaaaggatgcaaaagtgcttttccagaactgggggattgaacccatcgacagtgtaatgggaatgatcggaatattgcctgatatgaccccaagcacggaaatgtcactgagaggtataagagtcagcaaaatgggagtagatgaatattccagtacggagagagtggtagtgagcattgaccgatttttgagagttcgggatcaacgagggaacgtactattgtcccccgaagaggtcagcgagacacagggaactgagaaattgaccataacttattcgtcatcaatgatgtgggagatcaatggtcctgagtcagtgctggtcaacacttatcaatggatcataaggaactgggaaagcttgaaaattcaatggtcacaggatcccacgatgttatacaacaaaatggaatttgaaccattccagtctcttgtccctaaggcaaccagaagtcgttacagtggattcgtgaggacactgttccagcaaatgcgggatgtgcttggaacatttgatactgtccaaataataaagcttctcccctttgctgcagctccaccggaacagagtaggatgcagttctcctcgctgactgtgaatgtaagaggatcagggctgaggatactggtaagaggcaattctccagtgttcaattacaataaagcaaccaaaaggcttacaattcttggaaaagatgcaggtgcattgactgaagatccagatgaaggcacagctggagtggagtctgctgtcctgaggggattcctcattttgggtaaagaagacaagagatatggcccagcattaagcatcaatgaactgagcaatcttgcaaaaggagagaaggctaatgtgctaattgggcaaggagacgtggtgttggtaatgaaacggaaacgggactctagcatacttactgacagccagacagcgaccaaaaggattcggatggccatcaattag

SEQ ID NO: 2 - DNA, 2587 bp, Influenza A virus
  misc_feature (1)..(2587): Flu A 1 polymerase acidic (PA) gene (consensus sequence, KC471368.1 Influenza A virus A/Swine/Korea/ CY01-04/2012 (H1N1) segment 3)
atggaagactttgtgcgacaatgcttcaatccgatgatcgtcgagcttgcggaaaaggcaatgaaagaatatggggaagatccgaaaatcgaaactaacaagtttgctgcaatatgcacacatttggaagtttgtttcatgtattcggatttccatttcatcgacgaacggggtgaatcaataattgtagaatctggtgacccgaatgcactattgaagcaccgatttgagataattgaaggaagagaccgaatcatggcctggacagtggtgaacagtatatgtaacacaacaggggtagagaagcctaaatttcttcctgatttgtatgattacaaagaaaaccggttcattgaaattggagtaacacggagggaagtccacatatattacctagagaaagccaacaaaataaaatctgagaagacacacattcatggaagactttgtgcgacaatgcttcaatccgatgatcgtcgagcttgcggaaaaggcaatgaaagaatatggggaagatccgaaaatcgaaactaacaagtttgctgcaatatgcacacatttggaagtttgtttcatgtattcggatttccatttcatcgacgaacggggtgaatcaataattgtagaatctggtgacccgaatgcactattgaagcaccgatttgagataattgaaggaagagaccgaatcatggcctggacagtggtgaacagtatatgtaacacaacaggggtagagaagcctaaatttcttcctgatttgtatgattacaaagaaaaccggttcattgaaattggagtaacacggagggaagtccacatatattacctagagaaagccaacaaaataaaatctgagaagacacacattcacatcttttcattcactggagaggagatggccaccaaagcagactacacccttgacgaagagagcagggcaagaatcaaaactaggcttttcactataagacaagaaatggccagtaggagtctatgggattcctttcgtcaatccgaaagaggcgaagagacaattgaagaaaaatttgagattacaggaactatgcgcaagcttgccgaccaaagtctcccaccgaacttctccagccttgaaaactttagagcctatgtagatggattcgagccgaacggctgcattgagggcaagctttcccaaatgtcaaaggaagtgaacgccaaaattgaaccattcttgaggacgacaccacgccccctcagattgcctgatgggcctctttgccatcagcggtcaaagttcctgctgatggatgctctgaaattaagtattgaagacccgagtcacgagggagagggaataccactatatgatgcaatcaaatgcatgaagacattctttggctggaaagagcctaacatagtcaaaccacataagaaaggcataaatcccaattaccttatggcttggaagcaggtgctaacagagctacaggacattgaaaatgaagagaagatcccaaggacaaagaacatgaagagaacaagccaattgaagtgggcactcggtgaaaatatggcaccagaaaaagtagactttgatgactgcaaagatgttggagaccttaaacagtatgacagtgatgagccagagcccagatctctagcaagctgggtccaaaatgaattcaataaggcatgtgaattgactgattcaagctggatagaacttgatgaaataggagaagatgttgccccgattgaacatatcgcaagcatgaggaggaactattttacagcagaagtgtcccactgcagggctactgaatacataatgaagggagtgtacataaatacggccttgctcaatgcatcctgtgcagccatggatgactttcagctgatcccaatgataagcaaatgtaggaccaaagaaggaagacggaaaacaaacctgtatgggttcattataaaaggaaggtctcatttgagaaatgatactgatgtggtgaactttgtaagtatggagttctcactcactgacccgagactggagccacacaaatgggaaaaatactgtgttcttgaaataggagacatgctcttgaggactgcgataggccaagtgtcgaggcccatgttcctatatgtgagaaccaatggaacctccaagatcaagatgaaatggggcatggaaatgaggcgctgccttcttcagtcccttcagcagattgagagcatgattgaggccgagtcttctgtcaaagagaaagacatgaccaaggaattctttgaaaacaaatcagaaacatggccaatcggagagtcacccagaggagtggaggaaggctctattgggaaagtgtgcaggaccttactggcaaaatctgtgttcaacagtctatatgcgtctccacaacttgaggggttttcggctgaatcgagaaaattgcttctcattgttcaggcacttagggacaacctggaacctggaaccttcgatcttggggggctatatgaagcaatcgaggagtgcctgattaatgatccctgggttttgcttaatgcatcttggttcaactccttcctcacacatgcactgaagtag

SEQ ID NO: 3 - DNA, 982 bp, Influenza A virus
  misc_feature (1)..(982): Flu A 1 matrix protein (MP) gene (consensus sequence KC951136.1 of segment 7 from A/Swine/ Pennsylvania/ A01432652/2013 (H3N2))
atgagtcttctaaccgaggtcgaaacgtacgttctttctatcataccgtcaggccccctcaaagccgagatcgcgcagagactggaaagtgtctttgcaggaaagaacacagatcttgaggctctcatggaatggctaaagacaagaccaatcttgtcacctttgactaagggaattttaggatttgtgttcacgctcaccgtgcccagtgagcgaggactgcagcgtagacgctttgtccaaaatgccctaaatgggaatggggacccaaacaacatggatagagcagttaaactatacaagaagctcaaaagagaaataacgttccatggggccaaggaggtgtcactaagctattcaactggtgcacttgccagttgcatgggcctcatatacaacaggatgggaacagtgaccacagaagctgcttttggtctagtgtgtgccacttgtgaacagattgctgattcacagcatcggtctcacagacagatggctactaccaccaatccactaatcaggcatgagaacagaatggtgctggctagcactacggcaaaggctatggaacagatggctggatcgagtgaacaggcagcggaggccatggaggttgctaatcagactaggcagatggtacatgcaatgagaactattgggactcatcctagctccagtactggtctgaaagatgaccttcttgaaaatttgcaggcctaccagaagcgaatgggagtgcagatgcagcgattcaagtgatcctctcgccattgcagcaaatatcattgggatcttgcacctgatattgtggattactgatcgtctttttttcaaatgtatttatcgtcgctttaaatacggtttgaaaagagggccttctacagaaggagtgcctgagtccatgagggaagaatatcaacaggaacagcagagtgctgtggatgttgacgatggtcattttgtcaacatagagctagagtaa

SEQ ID NO: 4 - DNA, 931 bp, Influenza A virus
  misc_feature (1)..(931): Flu A 2 1 matrix protein (MP) gene (consensus sequence, KF018056.1 Influenza A virus A/Taiwan/ T02081/2013 (H7N9) segment 7)
cgtacgttctatctatcattccatcaggccccctcaaagccgagatcgcgcagagacttgaggatgtttttgcagggaagaacgcagatctcgaggctctcatggagtggataaagacaagaccaatcctgtcacctctgactaaggggattttagggtttgtgttcacgctcaccgtgcccagtgagcgaggactgcagcgtagacggtttgtccaaaacgccctaaatgggaatggagacccaaacaacatggacaaggcagttaaattatacaagaaactgaagagggaaatgacattccatggagcaaaggaagttgcactcagttactcaactggtgcgcttgccagctgcatgggtctcatatacaacaggatggggacagtaactgcagaaggggctcttggattggtatgtgccacttgtgagcagattgctgacgcacaacatcggtcccacaggcagatggcaactactaccaacccactaattaggcatgagaatagaatggtactagccagtactacggctaaggctatggagcagatggctggatcaagtgaacaggcagcggaagccatggaagttgcaagccaggctaggcaaatggtgcaggctatgagaacagtcgggactcaccctaactccagtacaggtctaaaggatgatcttattgaaaatttgcaggcttaccagaaccggatgggagtgcaactgcagcggttcaagtgatcctctcgttgttgcagctaacattattgggatattgcacttgatattgtggattcttgatcgtcttttcttcaaatgcatttatcgtcgctttaaatacggtttgaaaagagggccttctacggaaggaatgcctgagtctatgagggaagaatatcggcaggaacagcagaatgctgtggatgttgacgatggtc

SEQ ID NO: 5 - DNA, 1683 bp, Influenza A virus
  misc_feature (1)..(1683): Flu A 3 haemagglutinin (HA) gene (consensus sequence, KC896763.1 Influenza A virus A/Nanjing/ 2913/2013 (H7N9) segment 4)
atgaacactcaaatcctggtattcgctctgattgcgatcattccaacaaatgcagacaaaatctgcctcggacatcatgccgtgtcaaacggaaccaaagtaaacacattaactgaaagaggagtggaagtcgtcaatgcaactgaaacagtggaacgaacaaacatccccaggatctgctcaaaagggaaaatgacagttgacctcggtcaatgtggactcctggggacaatcactggaccacctcaatgtgaccaattcctagaattttcagccgatttaattattgagaggcgagaaggaagtgatgtctgttatcctgggaaattcgtgaatgaggaagctctgaggcaaatactcagagaatcaggcggaattgacaaggaagcaatgggattcacatacagtggaataagaactaatggagcaaccagtgcatgtaggagatcaggatcttcattctatgcagaaatgaaatggctcctgtcaaacacagataatgctgcattcccgcagatgactaagtcatataaaaatacaagaaaaagcccagctctaatagtatgggggatccatcattccgtatcaactgcagagcaaaccaagctatatgggagtggaaacaaactggtgacagttgggagttctaattatcaacaatcttttgtaccgagtccaggagcgagaccacaagttaatggtctatctggaagaattgactttcattggctaatgctaaatcccaatgatacagtcactttcagtttcaatggggctttcatagctccagaccgtgcaagcttcctgagaggaaaatctatgggaatccagagtggagtacaggttgatgccaattgtgaaggggactgctatcatagtggagggacaataataagtaacttgccatttcagaacatagatagcagggcagttggaaaatgtccgagatatgttaagcaaaggagtctgctgctagcaacagggatgaagaatgttcctgagattccaaagggaagaggcctatttggtgctatagcgggtttcattgaaaatggatgggaaggcctaattgatggttggtatggtttcagacaccagaatgcacagggagagggaactgctgcagattacaaaagcactcaatcggcaattgatcaaataacaggaaaattaaaccggcttatagaaaaaaccaaccaacaatttgagttgatagacaatgaattcaatgaggtagagaagcaaatcggtaatgtgataaattggaccagagattctataacagaagtgtggtcatacaatgctgaactcttggtagcaatggagaaccagcatacaattgatctggctgattcagaaatggacaaactgtacgaacgagtgaaaagacagctgagagagaatgctgaagaagatggcactggttgctttgaaatatttcacaagtgtgatgatgactgtatggccagtattagaaataacacctatgatcacagcaaatacagggaagaggcaatgcaaaatagaatacagattgacccagtcaaactaagcagcggctacaaagatgtgatactttggtttagcttcggggcatcatgtttcatacttctagccattgtaatgggccttgtcttcatatgtgtaaagaatggaaacatgcggtgcactatttgtatataa

SEQ ID NO: 6 - DNA, 1139 bp, Influenza B virus
  misc_feature (1)..(1139): Flu B matrix protein (MP) gene (consensus sequence, KC814126.1 Influenza B virus B/Utah/03/2011 segment 7
atgtcgctgtttggagacacaattgcctacctgctttcattgacagaggatggagaaggcaaagcagaactagcagaaaaattacactgttggtttggtgggaaagaatttgacctagactctgccttggaatggataaaaaacaaaagatgcttaactgatatacaaaaagcactaattggtgcctctatatgctttttaaaacccaaagaccaggaaagaaaaagaagattcatcacagagcccttatcaggaatgggaacaacagcaacaaaaaagaaaggcctgattctggctgagagaaaaatgagaagatgtgtgagctttcatgaagcatttgaaatagcagaaggccatgaaagctcagcgctactatactgtctcatggtcatgtacctgaatcctggaaattattcaatgcaagtaaaactaggaacgctctgtgctttatgcgagaaacaagcatcacattcacacagggctcatagcagagcagcgagatcttcagtgcctggagtgagacgagaaatgcagatggtctcagctatgaacacagcaaaaacaatgaatggaatgggaaaaggagaagacgtccaaaagctggcagaagagttgcaaagcaacattggagtgctgagatctcttggagcaagccaaaagaatggggaagggattgcaaaggatgtaatggaagtgctaaagcagagctccatgggaaattcagctcttgtgaagaaatatctataatgctcgaaccatttcagattcttacaatttgttcttttatcttatcagctctccatttcatggcttggacaatagggcatttgaatcaaataaaaagaggaataaacatgaaaatacgaataaaaggtccaaacaaagagacaataaacagagaggtatcaattttgagacacagttaccaaaaagaaatccaggccaaagaaacaatgaaggaagtactctctgacaacatggaggtattgaatgaccacataataattgaggggctttctgccgaagagataataaaaatgggtgaaacagttttggagatagaagaattgcattaaattcaattttactatatttcttactatgcatttaagcaaattgtaatcaatgtcagcaaataa

SEQ ID NO: 7 - DNA, 1054 bp, Influenza B virus
  misc_feature (1)..(1054): Flu B nonstructural (NS) gene (consensus sequence KC892145.1 Influenza B virus B/California/03/ 2012 segment 8
atggcgaacaacaacatgaccacaacacaaattgaggtgggtccgggagcaaccaatgccaccataaactttgaagcaggaattctggagtgctatgaaaggctttcatggcaaagagcccttgactaccccggtcaagaccgcctaaacagactaaagagaaaattagagtcaagaataaagactcacaacaaaagtgagcctgaaagtaaaaggatgtcccttgaagagagaaaagcaattggagtaaaaatgatgaaagtactcctatttatgaatccgtctgctggaattgaagggtttgagccatactgtatgaacagttcctcaaatagcaactgtacgaaatacaattggaccgattacccttcaacaccagagaggtgccttgatgacatagaggaagaaccagaggatgttgatggcccaactgaaatagtattaagggacatgaacaacaaagatgcaaggcaaaagataaaggaggaagtaaacactcagaaagaagggaagttccgtttgacaataaaaagggatatgcgtaatgtattgtccttgagagtgttggtaaatggaacattcctcaaacaccccaatggatacaagtccttatcaactctgcatagattgaatgcatatgaccagagtggaaggcttgttgctaaacttgttgccactgatgatcttacagtggaggatgaagaagatggccatcggatcctcaactcactcttcgagcgtcttaatgaaggacattcaaagccaattcgagcagctgaaactgcggtgggagtcttatcccaatttggtcaagagcaccgattatcaccagaagagggagacaattagactggtcacggaagaactttatcttttaagtaaaagaattgatgataacatactattccacaaaacagtgatagctaacagctccataatagctgacatggttgtatcattatcattattagaaacattgtatgaaatgaaggatgtggttgaagtgtacagcaggcagtgcttgtgaatttaaaataaaaatcctgttactact

SEQ ID NO: 8 - DNA, 68 bp, Artificial Sequence
  Synthetic: Flu A 1 PB2 amplicon
aaacgggactctagcatacttactgacagccagacagcgaccaaaaggattcggatggccatcaatta

SEQ ID NO: 9 - DNA, 88 bp, Artificial Sequence
  Synthetic: Flu A 1 PA amplicon
atcttggggggctatatgaagcaatcgaggagtgcctgattaatgatccctgggttttgcttaatgcatcttggttcaactccttcct

SEQ ID NO: 10 - DNA, 145 bp, Artificial Sequence
  Synthetic: Flu A 1 MP amplicon
ttctaaccgaggtcgaaacgtacgttctttctatcataccgtcaggccccctcaaagccgagatcgcgcagagactggaaagtgtctttgcaggaaagaacacagatcttgaggctctcatggaatggctaaagacaagaccaat

SEQ ID NO: 11 - DNA, 91 bp, Artificial Sequence
  Synthetic: Flu A 2 MP amplicon
caagaccaatcctgtcacctctgactaaggggattttagggtttgtgttcacgctcaccgtgcccagtgagcgaggactgcagcgtagacg

SEQ ID NO: 12 - DNA, 81 bp, Artificial Sequence
  Synthetic: Flu A 3 HA amplicon
gaaatgaaatggctcctgtcaaacacagataatgctgcattcccgcagatgactaagtcatataaaaatacaagaaaaagc

SEQ ID NO: 13 - DNA, 107 bp, Artificial Sequence
  Synthetic: Flu B MP amplicon
tttggagacacaattgcctacctgctttcattgacagaggatggagaaggcaaagcagaactagcagaaaaattacactgttggtttggtgggaaagaatttgacct

SEQ ID NO: 14 - DNA, 112 bp, Artificial Sequence
  Synthetic: Flu B NS amplicon
gatggccatcggatcctcaactcactcttcgagcgtcttaatgaaggacattcaaagccaattcgagcagctgaaactgcggtgggagtcttatcccaatttggtcaagagc

SEQ ID NO: 15 - DNA, 121 bp, Artificial Sequence
  Synthetic: RSV A amplicon
tacactcaacaaagatcaacttctgtcatccagcaaatacaccatccaacggagcacaggagatagtattgatactcctaattatgatgtgcagaaacacatcaacaagttatgtggcatg

SEQ ID NO: 16 - DNA, 120 bp, Artificial Sequence
  Synthetic: RSV B amplicon
cattaaataaggatcagctgctgtcatccagcaaatacactattcaacgtagtacaggagataatattgacactcccaattatgatgtgcaaaaacacctaaacaaactatgtggtatgc

SEQ ID NO: 17 - DNA, 20 bp, Artificial Sequence
  Synthetic: Flu A PB2 Fwd
aaacgggactctagcatact

SEQ ID NO: 18 - DNA, 20 bp, Artificial Sequence
  Synthetic: Flu A PB2 Rev
taattgatggccatccgaat

SEQ ID NO: 19 - DNA, 20 bp, Artificial Sequence
  Synthetic: Flu A PB2 FAM_Q38
agccagacagcgaccaaaag

SEQ ID NO: 20 - DNA, 25 bp, Artificial Sequence
  Synthetic: Flu A PA Fwd
atcttggggggctatatgaagcaat

SEQ ID NO: 21 - DNA, 20 bp, Artificial Sequence
  Synthetic: Flu A PA Rev
aggaaggagttgaaccaaga

SEQ ID NO: 22 - DNA, 20 bp, Artificial Sequence
  Synthetic: Flu A PA FAM_Q38
aatgatccctgggttttgct

SEQ ID NO: 23 - DNA, 20 bp, Artificial Sequence
  Synthetic: Flu A MP fwd
ttctaaccgaggtcgaaacg

SEQ ID NO: 24 - DNA, 20 bp, Artificial Sequence
  Synthetic: Flu A MP Rev
attggtcttgtctttagcca

SEQ ID NO: 25 - DNA, 20 bp, Artificial Sequence
  Synthetic: Flu A MP FAM_Q38
tcaggccccctcaaagccga

SEQ ID NO: 26 - DNA, 20 bp, Artificial Sequence
  Synthetic: Avian MP Fwd
caagaccaatcctgtcacct

SEQ ID NO: 27 - DNA, 20 bp, Artificial Sequence
  Synthetic: Avian MP Rev
cgtctacgctgcagtcctcg

SEQ ID NO: 28 - DNA, 20 bp, Artificial Sequence
  Synthetic: Avian MP CF5_Q38
acgctcaccgtgcccagtga

SEQ ID NO: 29-31 - skipped (3 entries)

SEQ ID NO: 32 - DNA, 20 bp, Artificial Sequence
  Synthetic: Flu B MP Fwd
ttggagacacgattgcctac

SEQ ID NO: 33 - DNA, 20 bp, Artificial Sequence
  Synthetic: Flu B MP Rev
aggtcaaattctttcccacc

SEQ ID NO: 34 - DNA, 20 bp, Artificial Sequence
  Synthetic: Flu B MP CF3_Q38
atggagaaggcaaagcagaa

SEQ ID NO: 35 - DNA, 20 bp, Artificial Sequence
  Synthetic: Flu B NS Fwd
gatggccatcggatcctcaa

SEQ ID NO: 36 - DNA, 20 bp, Artificial Sequence
  Synthetic: Flu B NS Rev
gctcttgaccaaattgggat

SEQ ID NO: 37 - DNA, 20 bp, Artificial Sequence
  Synthetic: Flu B NS CF3_Q38
aaagccaattcgagcagctg

SEQ ID NO: 38 - DNA, 27 bp, Artificial Sequence
  Synthetic: RSV A Fwd
tacactcaacaaagatcaacttctgtc

SEQ ID NO: 39 - DNA, 25 bp, Artificial Sequence
  Synthetic: RSV A Rev
catgccacataacttattgatgtgt

SEQ ID NO: 40 - DNA, 24 bp, Artificial Sequence
  Synthetic: RSV A CF4_Q38
caccatccaacggagcacaggaga

SEQ ID NO: 41 - DNA, 25 bp, Artificial Sequence
  Synthetic: RSV B Fwd
cattaaataaggatcagctgctgtc

SEQ ID NO: 42 - DNA, 27 bp, Artificial Sequence
  Synthetic: RSV B Rev
gcataccacatagtttgtttaggtgtt

SEQ ID NO: 43 - DNA, 29 bp, Artificial Sequence
  Synthetic: RSV B CF4_Q38
taatattgatactcccaattatgatgtgc

SEQ ID NO: 44 - DNA, 228 bp, Severe acute respiratory syndrome coronavirus 2
  misc_feature (1)..(228): SARS-CoV-2  E gene 26249  26476 nt of MT276598
atgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaa

SEQ ID NO: 45 - DNA, 1250 bp, Severe acute respiratory syndrome coronavirus 2
  misc_feature (1)..(1250): SARS-CoV-2 N2 gene 28278  29537 nt of MT276598
aactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaaacgaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctg

SEQ ID NO: 46 - DNA, 93 bp, Severe acute respiratory syndrome coronavirus 2
  misc_feature (1)..(93): SARS-CoV-2  E amplicon
tcggaagagacaggtacgttaatagttaatagcgtacttctttttcttgctttcgtggtattcttgctagttacactagccatccttactgcg

SEQ ID NO: 47 - DNA, 67 bp, Severe acute respiratory syndrome coronavirus 2
  misc_feature (1)..(67): SARS-CoV-2 N2 amplicon
ttacaaacattggccgcaaattgcacaatttgcccccagcgcttcagcgttcttcggaatgtcgcgc

SEQ ID NO: 48 - DNA, 20 bp, Artificial Sequence
  Synthetic: CEP-COV2-E-FWD1
tcggaagagacaggtacgtt

SEQ ID NO: 49 - DNA, 19 bp, Artificial Sequence
  Synthetic: CEP-COV2-E-REV1
cgcagtaaggatggctagt

SEQ ID NO: 50 - DNA, 21 bp, Artificial Sequence
  Synthetic: CEP-COV2-E-PRCF1-3
tgctttcgtggtattcttgct

SEQ ID NO: 51 - DNA, 20 bp, Artificial Sequence
  Synthetic: CEP-19NCOV-N2-FWD2
ttacaaacattggccgcaaa

SEQ ID NO: 52 - DNA, 19 bp, Artificial Sequence
  Synthetic: COV N2 Rev/CEP-19NCOV-N2-REV2
gcgcgacattccgaagaac

SEQ ID NO: 53 - DNA, 23 bp, Artificial Sequence
  Synthetic: CEP-19NCOV-N2-PRCF1-3
acaatttgcccccagcgcttcag

SEQ ID NO: 54 - DNA, 26 bp, Artificial Sequence
  Synthetic: COV2-E-W-F1
acaggtacgttaatagttaatagcgt

SEQ ID NO: 55 - DNA, 22 bp, Artificial Sequence
  Synthetic: COV2-E-W-R1
atattgcagcagtacgcacaca

SEQ ID NO: 56 - DNA, 26 bp, Artificial Sequence
  Synthetic: COV2-E-W-P2
acactagccatccttactgcgcttcg

SEQ ID NO: 57 - DNA, 20 bp, Artificial Sequence
  Synthetic: COV2-N2-C-F1
ttacaaacattggccgcaaa

SEQ ID NO: 58 - DNA, 18 bp, Artificial Sequence
  Synthetic: COV2-N2-C-R1
gcgcgacattccgaagaa

SEQ ID NO: 59 - DNA, 23 bp, Artificial Sequence
  Synthetic: COV2-N2-C-P1
acaatttgcccccagcgcttcag

SEQ ID NO: 60 - DNA, 22 bp, Artificial Sequence
  Synthetic: W-19NCOV-FWD1
gtgaratggtcatgtgtggcgg

SEQ ID NO: 61 - DNA, 28 bp, Artificial Sequence
  Synthetic: COV2 RDRP Rev/ RDRP-W-R2
caaatgttaaaaacactattagcataag

SEQ ID NO: 62 - DNA, 25 bp, Artificial Sequence
  Synthetic: RDRP-W-P2_Q13
caggtggaacctcatcaggagatgc

SEQ ID NO: 63 - DNA, 19 bp, Artificial Sequence
  Synthetic: CEP-19NCOV-ORF1AB-FWD11
gacaaatgctggtgattac

SEQ ID NO: 64 - DNA, 22 bp, Artificial Sequence
  Synthetic: CEP-COV-ORF1AB-REV
ctttgagcgtttctgctgcaaa

SEQ ID NO: 65 - DNA, 27 bp, Artificial Sequence
  Synthetic: CEP-COV-ORF1AB-PRCF1-3
ctaacacctgtactgaaagactcaagc

SEQ ID NO: 66 - DNA, 115 bp, Artificial Sequence
  Synthetic: SARS-CoV-2  E Alt amplicon (COV2-E-W)
agacaggtacgttaatagttaatagcgtacttctttttcttgctttcgtggtattcttgctagttacactagccatccttactgcgcttcgattgtgtgcgtactgctgcaatat

SEQ ID NO: 67 - DNA, 27 bp, Artificial Sequence
  Synthetic: RSV A V2 Fwd
tacactcaacaaagatcaacttctatc

SEQ ID NO: 68 - DNA, 25 bp, Artificial Sequence
  Synthetic: RSV A V2a Rev
catgccacataacttattaatgtgt

SEQ ID NO: 69 - DNA, 25 bp, Artificial Sequence
  Synthetic: RSV A V2b Rev
catgccacatagcttattgatgtgt

SEQ ID NO: 70 - DNA, 20 bp, Artificial Sequence
  Synthetic: COV E V2 Fwd
gctttcgtggtattcttgct

SEQ ID NO: 71 - DNA, 18 bp, Artificial Sequence
  Synthetic: COV E V2 Rev
gcagtacgcacacaatcg

SEQ ID NO: 72 - DNA, 21 bp, Artificial Sequence
  Synthetic: COV E V2 P
cactagccatccttactgcgc

SEQ ID NO: 73 - DNA, 26 bp, Artificial Sequence
  Synthetic: COV N2 V2 Fwd
aaaaaattacaaacattggccgcaaa

SEQ ID NO: 74 - DNA, 24 bp, Artificial Sequence
  Synthetic: COV N2 V2 P1
cacaatttgcccccagcgcttcag

SEQ ID NO: 75 - DNA, 24 bp, Artificial Sequence
  Synthetic: COV N2 V2 P2
cacaatttgccccuagcgcttcag

SEQ ID NO: 76 - DNA, 20 bp, Artificial Sequence
  Synthetic: COV2 RDRP V2 Fwd
cacttgttcttgctcgcaaa

SEQ ID NO: 77 - DNA, 21 bp, Artificial Sequence
  Synthetic: COV2 RDRP V2 P
caacgtgttgtagcttgtcac

SEQ ID NO: 78 - DNA, 25 bp, Artificial Sequence
  Synthetic: COV2 RDRP V2 Rev2
ctcattagctaatctatagaaacgg

SEQ ID NO: 79 - DNA, 66 bp, Artificial Sequence
  Synthetic: SARS-CoV-2  E Alt amplicon
gctttcgtggtattcttgctagttacactagccatccttactgcgcttcgattgtgtgcgtactgc

SEQ ID NO: 80 - DNA, 187 bp, Artificial Sequence
  Synthetic: RDRP amplicon 1
caaatgttaaaaacactattagcataagcagttgtggcatctcctgatgaggttccacctggtttaacatatagtgaaccgccacacatgaccatttcactcaatacttgagcacactcattagctaatctatagaaacggtgtgacaagctacaacacgttgtatgtttgcgagcaagaacaagtg

SEQ ID NO: 81 - DNA, 71 bp, Artificial Sequence
  Synthetic: RDRP amplicon 2
ctcattagctaatctatagaaacggtgtgacaagctacaacacgttgtatgtttgcgagcaagaacaagtg